(19) United States
(12) Patent Application Publication
(10) Pub. No.: US 2025/0269020 A1
(43) Pub. Date: Aug. 28, 2025

(54) STABLE FORMULATIONS OF ANTI-ILT4 ANTIBODIES OR ANTIGEN-BINDING FRAGMENTS THEREROF IN COMBINATION WITH ANTI-PD-1 ANTIBODIES AND METHODS OF USE THEREOF

(71) Applicant: Merck Sharp & Dohme LLC, Rahway, NJ (US)

(72) Inventors: Nicole L. Buist, Holmdel, NJ (US); Ashlesha S. Raut, Mountainside, NJ (US); Marco A. Blanco, Maplewood, NJ (US); Monisha Dey, Chatham, NJ (US); Sachin Mittal, Bridgewater, NJ (US)

(73) Assignee: Merck Sharp & Dohme LLC, Rahway, NJ (US)

(21) Appl. No.: 18/859,877

(22) PCT Filed: Apr. 25, 2023
(86) PCT No.: PCT/US2023/019705

§ 371 Date: Oct. 24, 2024

Related U.S. Application Data
(60) Provisional application No. 63/336,839, filed on Apr. 29, 2022.

(51) Int. Cl. A61K 39/395; A61K 9/00; A61K 9/08; A61K 39/00; A61K 47/18; A61K 47/22; A61K 47/26

(52) U.S. Cl. CPC A61K 39/39558; A61K 9/0019; A61K 9/08; A61K 39/39591; A61K 47/183; A61K 47/22; A61K 47/26; A61K 2039/507

(57) ABSTRACT

The invention relates to stable coformulations of antibodies or antigen-binding fragments thereof that bind to human immunoglobulin-like transcript 4 (ILT4) and PD-1. Also provided are methods of treating various cancers using the formulations disclosed herein.

### CROSS-REFERENCE TO RELATED APPLICATIONS

This application claims the benefit of priority to U.S. Provisional Application No. 63/336,839, filed Apr. 29, 2022, the disclosure of which is incorporated herein by its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 17, 2022, is named 25400-WO-PCT_SL.xml, and is 99,833 bytes in size.

### FIELD OF THE INVENTION

The invention relates generally to pharmaceutical formulations of an antibody or antigen-binding fragment thereof that binds to human interleukin-like transcript 4 (ILT4) and their use in treating various disorders (e.g., cancer).

### BACKGROUND OF THE INVENTION

A common strategy used by tumor cells to escape innate and adaptive immune response is associated with aberrant expression of human leukocyte antigen (HLA)-G (Curigliano et al. Clin Cancer Res. 2013; 19(20): 5564-5571 and Gonzalez et al. Crit Rev Clin Lab Sci. 2012; 49(3): 63-84). HLA-G can directly inhibit immune cell function through receptor binding and/or through trogocytosis and impairment of chemotaxis (Morandi et al. Cytokine Growth Factor Review. 2014, 25: 327-335 and Lin et al. Mol Med. 2015, 21: 782-791). High expression of HLA-G in multiple tumor types, including for example, colorectal, pancreatic, endometrial, lung, breast, ovarian, and gastric cancer, is associated with advanced disease stage, tumor invasiveness, metastatic potential and an unfavorable prognosis (Lin et al. Mol Med. 2015, 21: 782-791 and Loumange et al. Int J Cancer. 2014, 135, 222: 581-597). Antibody-mediated blockade of HLA-G function in transgenic mouse models has been shown to inhibit tumor development and block expansion of myeloid-derived suppressor cells (MDSC) (Loumange et al. Int J Cancer. 2014, 135, 222: 581-597, Lin et al. Hum Immunol. 2013, 74: 439-446, and Agaugue et al. Blood. 2011, 117: 7021-7031). HLA-G binding to ILT4 can directly inhibit the function of monocytes, dendritic cells, and neutrophils, thus impairing the innate immune anti-tumor response. The interaction between HLA-G and monocytes due to ILT4 inhibits maturation of human monocyte-derived antigen-presenting cells (APCs) resulting in a reduced expression of MHC class II antigens and co-stimulatory molecules through Stat3 activation (Colonna et al. J Immunol. 1998, 160: 3096-3100; Allan et al. J Exp Med. 1999, 189(7): 1149-1155, and Liang et al. Proc Natl Sci USA. 2008, 105(24): 8357). Using human monocyte-derived dendritic cells (DCs) and ILT4-transgenic mice, HLA-G was shown to induce the development of tolerogenic APCs with arrest maturation/activation of myeloid DCs, and the induction of tolerogenic DCs by HLA-G was through disrupting the MHC class II presentation pathway (Ristich et al. Eur J Immunol. 2005, 35: 1133-1142).

PD-1 is recognized as an important molecule in immune regulation and the maintenance of peripheral tolerance. PD-1 is moderately expressed on naive T, B and NKT cells and upregulated by T/B cell receptor signaling on lymphocytes, monocytes and myeloid cells. Two known ligands for PD-1, PD-L1 (B7-H1) and PD-L2 (B7-DC), are expressed in human cancers arising in various tissues. In large sample sets of e.g., ovarian, renal, colorectal, pancreatic, liver cancers and melanoma, it was shown that PD-L1 expression correlated with poor prognosis and reduced overall survival irrespective of subsequent treatment. Similarly, PD-1 expression on tumor infiltrating lymphocytes was found to mark dysfunctional T cells in breast cancer and melanoma and to correlate with poor prognosis in renal cancer. Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 are FDA approved for treating cancer. It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents.

As a consequence, the need exists for stable co-formulations of an anti-ILT4 antibody and an anti-PD-1 antibody. Such stable formulations will preferably exhibit stability over months to years under conditions typical for storage of drugs for self-administration, i.e., at refrigerator temperature in a syringe, resulting in a long shelf-life for the corresponding drug product.

### SUMMARY OF THE INVENTION

In one aspect, the invention provides a co-formulation of anti-human ILT4 (anti-ILT4) antibody or antigen-binding fragment thereof and anti-PD-1 antibodies or antigen binding fragments thereof. In one formulation, the co-formulation comprises: (i) about 10 mg/mL to about 200 mg/mL of the anti-ILT4 antibody or antigen-binding fragment thereof; (ii) about 10 mg/mL to about 200 mg/mL of an anti-PD-1 antibody or antigen binding fragment thereof; (iii) about 5 mM to about 20 mM of a buffer; (iv) about 6% to about 8% weight/volume (w/v) of a non-reducing sugar; (v) about 0.01% to about 0.10% (w/v) of a non-ionic surfactant; and (vi) about 1 mM to about 20 mM of an anti-oxidant, wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21), wherein the anti-PD-1 antibody or antigen-binding fragment thereof comprises a variable light chain region comprising CDRL1 of SEQ ID NO: 195, CDRL2 of SEQ ID NO: 296, and CDRL3 of SEQ ID NO: 397, and a variable heavy chain region comprising CDRH1 of SEQ ID NO: 6100, CDRH2 of SEQ ID NO: 7101, and CDRH3 of SEQ ID NO: 8102, wherein the buffer is an L-histidine buffer, an acetate buffer, or a citrate buffer, wherein the non-reducing sugar is a disaccharide, wherein the non-ionic surfactant is polysorbate 20 or polysorbate 80, and wherein the antioxidant is methionine.

In some embodiments, the non-reducing sugar is sucrose. In some embodiments, the buffer is L-histidine buffer. In some embodiments, the buffer is L-histidine buffer at a pH of about 5.0-6.5. In certain embodiments, the non-ionic surfactant is polysorbate 80 (PS-80). In some embodiments, the anti-oxidant is L-methionine.

Thus, in another aspect, provided herein is a formulation for an anti-ILT4 antibody or antigen-binding fragment thereof, comprising: (i) about 10 mg/mL to about 200 mg/mL of the anti-ILT4 antibody or antigen-binding fragment thereof; (ii) about 10 mg/mL to about 200 mg/mL of an anti-PD-1 antibody or antigen-binding fragment thereof; (iii) about 5 mM to about 20 mM L-histidine buffer; (iv) about 6% to about 8% (w/v) sucrose; (v) about 0.01% to about 0.10% (w/v) PS-80; and (vi) about 1 mM to about 20 mM L-methionine, wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21) and wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 102.

In some embodiments, the formulation comprises about 8 mM to about 12 mM L-histidine buffer.

In certain embodiments, the formulation comprises about 5 mM to about 10 mM L-methionine.

In other embodiments, the formulation comprises about 0.01% to about 0.05% (w/v) PS-80.

In yet other embodiments, the formulation comprises about 10 mg/mL to about 150 mg/mL of the anti-ILT4 antibody or antigen-binding fragment thereof. In still other embodiments, the concentration of the anti-ILT4 antibody or antigen-binding fragment thereof is about 10 mg/mL, about 12.5 mg/mL, about 15 mg/mL, about 25 mg/mL, about 50 mg/mL, about 75 mg/mL, about 100 mg/mL, about 125 mg/mL, or about 150 mg/mL. In one embodiment, the concentration of the anti-ILT4 antibody or antigen-binding fragment thereof is about 10 mg/mL. In another embodiment, the concentration of the anti-ILT4 antibody or antigen-binding fragment thereof is about 12.5 mg/mL. In yet another embodiment, the concentration of the anti-ILT4 antibody or antigen-binding fragment thereof is about 15 mg/mL. In still another embodiment, the concentration of the anti-ILT4 antibody or antigen-binding fragment thereof is about 25 mg/mL. In one embodiment, the concentration of the anti-ILT4 antibody or antigen-binding fragment thereof is about 50 mg/mL. In another embodiment, the concentration of the anti-ILT4 antibody or antigen-binding fragment thereof is about 75 mg/mL. In yet another embodiment, the concentration of the anti-ILT4 antibody or antigen-binding fragment thereof is about 100 mg/mL. In still another embodiment, the concentration of the anti-ILT4 antibody or antigen-binding fragment thereof is about 125 mg/mL. In yet still another embodiment, the concentration of the anti-ILT4 antibody or antigen-binding fragment thereof is about 150 mg/mL.

Thus, in one specific embodiment, the formulation comprises about 25 mg/mL of the anti-ILT4 antibody or antigen-binding fragment thereof, about 10 mM L-histidine buffer, about 7% w/v sucrose, about 0.025% polysorbate 80, and about 10 mM L-methionine.

In another specific embodiment, the formulation comprises about 50 mg/mL of the anti-ILT4 antibody or antigen-binding fragment thereof, about 10 mM L-histidine buffer, about 7% w/v sucrose, about 0.025% polysorbate 80, and about 10 mM L-methionine.

In yet another specific embodiment, the formulation comprises about 75 mg/mL of the anti-ILT4 antibody or antigen-binding fragment thereof, about 10 mM L-histidine buffer, about 7% w/v sucrose, about 0.025% polysorbate 80, and about 10 mM L-methionine.

In still another specific embodiment, the formulation comprises about 100 mg/mL of the anti-ILT4 antibody or antigen-binding fragment thereof, about 10 mM L-histidine buffer, about 7% w/v sucrose, about 0.025% polysorbate 80, and about 10 mM L-methionine.

In yet still another specific embodiment, the formulation comprises about 125 mg/mL of the anti-ILT4 antibody or antigen-binding fragment thereof, about 10 mM L-histidine buffer, about 7% w/v sucrose, about 0.025% polysorbate 80, and about 10 mM L-methionine.

In certain embodiments of various formulations provided herein, the formulation has a pH range of from about 5.0 to about 6.8. In some embodiments, the formulation has a pH range of from about 5.5 to about 6.0. In other embodiments, the formulation has a pH of about 5.5, about 5.6, about 5.7, about 5.8, about 5.9, or about 6.0. In one embodiment, the formulation has a pH of about 5.5. In another embodiment, the formulation has a pH of about 5.6. In yet another embodiment, the formulation has a pH of about 5.7. In still another embodiment, the formulation has a pH of about 5.8. In another embodiment, the formulation has a pH of about 5.9. In yet still another embodiment, the formulation has a pH of about 6.0.

Thus, in one specific embodiment, the pharmaceutical formulation for an anti-ILT4 antibody or antigen-binding fragment thereof comprises: (i) about 50 mg/mL to about 100 mg/mL of the anti-ILT4 antibody or antigen-binding fragment thereof; (ii) about 10 mg/mL to about 200 mg/mL of an anti-PD-1 antibody or antigen-binding fragment thereof; (iii) about 10 mM L-histidine buffer, pH about 5.5; (iv) about 7% (w/v) sucrose; (v) about 0.025% (w/v) polysorbate 80; and (vi) about 10 mM L-methionine, wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21), and wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 10.

In certain embodiments of the various formulations provided herein, the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX₁X₂NRPS, wherein X₁ is N, Q, E or D and X₂ is S or A (SEQ ID NO: 20), and CDR-L3: QSFDNSLSAYV (SEQ ID NO: 21).

In some embodiments of the various formulations provided herein, the anti-ILT4 antibody or antigen binding fragment thereof comprises a heavy chain variable domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GDSNRPS (SEQ ID NO: 52), and CDR-L3: QSFDNSLSAYV (SEQ ID NO: 21).

In other embodiments of the various formulations provided herein, the anti-ILT4 antibody or antigen binding fragment thereof comprises a heavy chain variable domain comprising the amino acid sequence set forth in SEQ ID NO:57 and a light chain variable domain comprising the amino acid sequence set forth in SEQ ID NO:58.

In yet other embodiments of the various formulations provided herein, the anti-ILT4 antibody or antigen binding fragment thereof comprises a heavy chain comprising the amino acid sequence set forth in SEQ ID NO:2 and a light chain comprising the amino acid sequence set forth in SEQ ID NO:7.

In still other embodiments of the various formulations provided herein, the anti-ILT4 antibody or antigen binding fragment thereof comprises a heavy chain comprising the amino acid sequence set forth in SEQ ID NO:80 and a light chain comprising the amino acid sequence set forth in SEQ ID NO:7.

In certain embodiments of the various formulations provided herein, the anti-ILT4 antibody or antigen binding fragment thereof comprises a heavy chain variable domain consisting of the amino acid sequence set forth in SEQ ID NO:57 and a light chain variable domain consisting of the amino acid sequence set forth in SEQ ID NO:58.

In some embodiments of the various formulations provided herein, the anti-ILT4 antibody or antigen binding fragment thereof comprises a heavy chain consisting of the amino acid sequence set forth in SEQ ID NO:2 and a light chain consisting of the amino acid sequence set forth in SEQ ID NO:7.

In certain embodiments of the various formulations provided herein, the anti-ILT4 antibody or antigen binding fragment thereof is a monoclonal antibody comprising two heavy chains comprising the amino acid sequence set forth in SEQ ID NO:2 and two light chains comprising the amino acid sequence set forth in SEQ ID NO:7.

In certain embodiments of the various formulations provided herein, the anti-ILT4 antibody or antigen binding fragment thereof is a monoclonal antibody comprising two heavy chains consisting of the amino acid sequence set forth in SEQ ID NO:2 and two light chains consisting of the amino acid sequence set forth in SEQ ID NO:7.

In other embodiments of the various formulations provided herein, the anti-ILT4 antibody or antigen binding fragment thereof comprises a heavy chain consisting of the amino acid sequence set forth in SEQ ID NO:80 and a light chain consisting of the amino acid sequence set forth in SEQ ID NO:7.

In certain embodiments of the various formulations provided herein, the anti-ILT4 antibody or antigen binding fragment thereof is a monoclonal antibody comprising two heavy chains comprising the amino acid sequence set forth in SEQ ID NO:80 and two light chains comprising the amino acid sequence set forth in SEQ ID NO:7.

In certain embodiments of the various formulations provided herein, the anti-ILT4 antibody or antigen binding fragment thereof is a monoclonal antibody comprising two heavy chains consisting of the amino acid sequence set forth in SEQ ID NO:80 and two light chains consisting of the amino acid sequence set forth in SEQ ID NO:7.

In one embodiment, the formulation is a liquid formulation. In one embodiment, the formulation is a frozen formulation. In another embodiment, the liquid formulation is stored at about 5° C. In yet another embodiment, the formulation is stored frozen at about −20° C. or below. In yet another embodiment, the formulation is stored frozen at about −70° C. or below. In still another embodiment, the liquid formulation is a reconstituted solution from a lyophilized formulation.

In certain embodiments, after the formulation is stored at about 3° C. to about 5° C. (e.g., 3° C., 4° C., 5° C.) for up to 9 months, (i) the % monomer of the anti-ILT4 antibody or antigen-binding fragment thereof is at least about 99% as determined by ultra-performance size exclusion chromatography; (ii) the % monomer of the anti-PD-1 antibody or antigen-binding fragment thereof is at least about 98% as determined by ultra-performance size exclusion chromatography; (iii) the turbidity of the formulation is at most about 0.135 as measured by OD₃₅₀₋₅₀₀; (iv) the % main peak of the anti-ILT4 antibody or antigen-binding fragment thereof is at least about 52%, the % acidic variant of the anti-ILT4 antibody or antigen-binding fragment thereof is at most about 15%, and/or the basic variant of the anti-ILT4 antibody or antigen-binding fragment thereof is at most about 12%, as determined by high performance ion-exchange chromatography; (v) the % main peak of the anti-PD-1 antibody or antigen-binding fragment thereof is at least about 10%, the % acidic variant of the anti-ILT4 antibody or antigen-binding fragment thereof is at most about 5%, and/or the basic variant of the anti-ILT4 antibody or antigen-binding fragment thereof is at most about 4%, as determined by high performance ion-exchange chromatography; (vi) the subvisible particle count of particles that are at least 2 μm in size is at most about 3500 as determined by microflow imaging; and/or (vii) the % oxidation of one or more amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping.

In some embodiments, after the formulation is stored at about 3° C. to about 5° C. for 6 months, the % monomer of the anti-ILT4 antibody or antigen-binding fragment thereof is at least about 99% as determined by ultra-performance size exclusion chromatography.

In some embodiments, after the formulation is stored at about 3° C. to about 5° C. for 6 months, the turbidity of the formulation is at most about 0.135 as measured by OD₃₅₀₋₅₀₀.

In some embodiments, after the formulation is stored at about 3° C. to about 5° C. for 6 months, the % main peak of the anti-ILT4 antibody or antigen-binding fragment thereof is at least about 63%, as determined by high performance ion-exchange chromatography. In some embodiments, after the formulation is stored at about 3° C. to about 5° C. for 6 months, the % acidic variant of the anti-ILT4 antibody or antigen-binding fragment thereof is at most about 23%, as determined by high performance ion-exchange chromatography. In some embodiments, after the formulation is stored at about 3° C. to about 5° C. for 6 months, the basic variant of the anti-ILT4 antibody or antigen-binding fragment thereof is at most about 14%, as determined by high performance ion-exchange chromatography. In some embodiments, after the formulation is stored at about 3° C. to about 5° C. for 6 months, the % main peak of the anti-ILT4 antibody or antigen-binding fragment thereof is at least about 63%, the % acidic variant of the anti-ILT4 antibody or antigen-binding fragment thereof is at most about 23%, and the basic variant of the anti-ILT4 antibody or antigen-binding fragment thereof is at most about 14%, as determined by high performance ion-exchange chromatography.

In some embodiments, after the formulation is stored at about 3° C. to about 5° C. for 6 months, the subvisible particle count of particles that are at least 2 μm in size is at most about 3500 as determined by microflow imaging.

In some embodiments, after the formulation is stored at about 3° C. to about 5° C. for 6 months, the % oxidation of one or more amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, the amino acid residue is W7 in the heavy chain of the anti-ILT4 antibody as set forth in SEQ ID NO:2 or 80. In another embodiment, the amino acid residue is W102 in the heavy chain of the anti-ILT4 antibody as set forth in SEQ ID NO:2 or 80. In yet another embodiment, the amino acid residue is M253 in the heavy chain of the anti-ILT4 antibody as set forth in SEQ ID NO:2 or 80. In still another embodiment, the amino acid residue is M359 in the heavy chain of the anti-ILT4 antibody as set forth in SEQ ID NO:2 or 80. In yet still another embodiment, the amino acid residue is M429 in the heavy chain of the anti-ILT4 antibody as set forth in SEQ ID NO:2 or 80.

In one embodiment, after the formulation is stored at about 3° C. to about 5° C. for 6 months, the % oxidation of one amino acid residue selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of two amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of three amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of four amino acid residues selected from the group consisting of W7, W102, M253. M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of all five amino acid residues from the group consisting of W7, W102, M253, M359, and M429 in the heavy chain of the anti-ILT4 antibody as set forth in SEQ ID NO:2 or 80 is less than about 4%, as determined by reduced peptide mapping.

In yet another aspect, provided herein is a method of treating a cancer in a human patient in need thereof, comprising administering a therapeutically effective amount of the pharmaceutical formulation described herein.

In still another aspect, provided herein is use of the pharmaceutical formulation described herein for preparing a medicament for treating a cancer.

### BRIEF DESCRIPTION OF THE DRAWINGS

FIG. 1 shows high molecular weight (HMW) species as determined by UP-SEC in anti-ILT4/anti-PD-1 co-formulation, anti-ILT4 SE and pembrolizumab SE samples at different concentrations of L-Methionine and for different levels of light exposure and residual metal, as described in Example 6.

FIG. 2 shows PS80 content as determined by Reverse Phase Chromatography in anti-ILT4/anti-PD-1 co-formulation, anti-ILT4 SE and pembrolizumab SE samples at different concentrations of L-Methionine and for different levels of light exposure and residual metal, as described in Example 6.

FIG. 3 shows oxidation of M105 as determined by Reduced Peptide Mapping on anti-ILT4/anti-PD-1 co-formulation samples with different L-Methionine concentrations (7-15 mM) at 40° C. for 4 weeks and as a function of metal concentration and light exposure, as described in Example 6.

FIG. 4 shows oxidation of W102 as determined by Reduced Peptide Mapping on anti-ILT4/anti-PD-1 co-formulation and anti-ILT4 SE samples with different L-Methionine concentrations (7-15 mM) at 40° C. for 4 weeks and as a function of metal concentration and light exposure, as described in Example 6.

FIG. 5 shows Oxidation of W7 as determined by Reduced Peptide Mapping on anti-ILT4/anti-PD-1 co-formulation and anti-ILT4 SE samples with different L-Methionine concentrations (7-15 mM) at 40° C. for 4 weeks and as a function of metal concentration and light exposure, as described in Example 6.

FIG. 6 shows total oxidation of Methionine residues as determined by Hydrophobic-Interaction Chromatography on pembrolizumab SE samples with different L-Methionine concentrations (7-15 mM) at 40° C. for 4 weeks and as a function of metal concentration and light exposure, as described in Example 6.

FIG. 7 shows contour plots with predicted values of HMW coformulations at different factor levels at 40° C., as described in Example 7.

FIG. 8 shows contour plots with predicted values of HMW at different factor levels for anti-ILT4 SE at 40° C., as described in Example 7.

FIG. 9 shows contour plots with predicted values of HMW at different factor levels for pembrolizumab SE at 40° C., as described in Example 7.

FIG. 10 shows contour plots with predicted values of % Acidic Variants for anti-ILT4 in the co-formulation at different factor levels at 40° C. for 4 weeks, as described in Example 7.

FIG. 11 shows the contour plot of the % Main for anti-ILT4 in the co-formulation as a function of different factors (L-Methionine concentration. Light exposure and Metal content). The figure illustrates that increase in Light stress decreases % Main for anti-ILT4, as described in Example 7.

FIG. 12 shows the contour plot of the % Basic Variants for anti-ILT4 in the co-formulation as a function of different factors (L-Methionine concentration, Light exposure and Metal content). The figure illustrates that increase in Light stress increases % Basic Variants for anti-ILT4, as described in Example 7.

FIG. 13 shows the contour plots with the predicted values from the model fits over different settings of the three parameters (L-Methionine concentration, Light exposure, Metal content) over time, as described in Example 7.

FIG. 14 shows the contour plot of the % Main for pembrolizumab in the co-formulation as a function of different factors (L-Methionine concentration, Light exposure, and Metal content), as described in Example 7.

FIG. 15 shows the contour plot of the % Basic Variants for pembrolizumab in the co-formulation as a function of different factors (L-Methionine concentration, Light exposure, and Metal content), as described in Example 7.

FIG. 16 shows the contour plots with the predicted values from the model fits over different settings of the three parameters (L-Methionine concentration, Light exposure, Metal content) over time, as described in Example 7.

FIG. 17 shows the contour plot of the % Main for anti-ILT4 in the anti-ILT4 SE formulation as a function of different factors (L-Methionine concentration, Light exposure and Metal content), as described in Example 7.

FIG. 18 shows the contour plot of the % Basic Variants for anti-ILT4 in the anti-ILT4 SE formulation as a function of different factors (L-Methionine concentration, Light exposure and Metal content), as described in Example 7.

FIG. 19 shows the L-Methionine concentration at all stability time points for all the three formulations, as described in Example 7.

FIG. 20 shows the plot of turbidity values for all the three formulations at 5° C. and 40° C. conditions, as described in Example 8.

FIGS. 21-25 show the raw data from peptide mapping for the SE and coformulation at initial and 40° C. conditions, as described in Example 8. FIG. 21 depicts M253 (Shared), FIG. 22 depicts M429 (Shared), FIG. 23 depicts W7 (anti-ILT4), FIG. 24 depicts W102 (anti-ILT4), and FIG. 25 depicts M105 (pembrolizumab).

FIG. 26 shows high molecular weight (HMW) and low molecular weight (LMW) species as determined by UP-SEC in coformulation 1 (CF1) and coformulation 2 (CF 2) when stored at 5° C. (circle) for 12 months, 25° C. (triangle pointing up) and 40° C. (triangle pointing down) for 6 months, as described in Example 8.

FIG. 27 shows anti-ILT4 and pembrolizumab charged variants as determined by IEX in CF 1 and CF 2 when stored at 5° C. (circle) for 12 months, 25° C. (triangle pointing up) and 40° C. (triangle pointing down) for 6 months, as described in Example 8.

FIG. 28 shows total L-Methionine content in CF 1 and CF 2 when stored at 5° C. (circle) for 12 months, 25° C. (triangle pointing down) and 40° C. (triangle pointing up) for 6 months, as described in Example 8.

FIG. 29 shows total PS-80 content in CF1 and CF2 when stored at 5° C. (circle) for 12 months, 25° C. (triangle pointing up) and 40° C. (triangle pointing down) for 6 months, as described in Example 8.

FIG. 30 shows sub-visible particle count in CF1 and CF2 when stored at 5° C. (light gray) for 12 months, 25° C. (dark gray) and 40° C. (black) for 6 months, as described in Example 8.

FIG. 31 shows oxidation of M105 (anti-PD-1), M429 (shared residue) and M253 (shared residue) as determined by Reduced Peptide Mapping CF 1 and CF 2 when stored at 5° C. (circle) for 12 months, 25° C. (triangle pointing up) and 40° C. (triangle pointing down) for 6 months, as described in Example 8

FIG. 32 shows oxidation of W102 (anti-ILT4 monoclonal antibody) and W7 (anti-ILT4 monoclonal antibody) as determined by Reduced Peptide Mapping CF 1 and CF 2 when stored at 5° C. (circle) for 12 months, 25° C. (triangle pointing up) and 40° C. (triangle pointing down) for 6 months, as described in Example 8.

### DETAILED DESCRIPTION OF THE INVENTION

### Definitions and Abbreviations

As used throughout the specification and appended claims, the following abbreviations apply:
AAPH 2,2′-Azobis(2-methylpropionamidine) dihydrochloride
API active pharmaceutical ingredient
CDR complementarity determining region in the immunoglobulin variable regions, defined using the Kabat numbering system, unless otherwise indicated
CE-SDS capillary electrophoresis sodium dodecyl sulfate
CHO Chinese hamster ovary
cIEF capillary isoelectric focusing
DLS dynamic light scattering test
DSC differential scanning calorimetry
DSF differential scanning fluorimetry
ELISA enzyme-linked immunosorbent assay
FR framework region
HC heavy chain
HMW high molecular weight
HP-IEX high performance ion-exchange chromatography
ICH International Council on Harmonization
IgG immunoglobulin G
LC liquid chromatography
LC-MS liquid chromatography-mass spectrometry
LMW low molecular weight
mAb monoclonal antibody
NR-CE-SDS non-reduced capillary electrophoresis sodium dodecyl sulfate
PS20 (or PS-20) polysorbate 20
PS80 (or PS-80) polysorbate 80
R-CE-SDS reducing capillary electrophoresis sodium dodecyl sulfate
RH relative humidity
SE single entity
t-BHP tert-butyl hydroperoxide
UP-SEC ultra-performance size exclusion chromatography
V_{H} immunoglobulin heavy chain variable region
V_{L} immunoglobulin light chain variable region
v/v volume per volume
WFI water for injection
w/v weight per volume

Unless specifically defined elsewhere in this document, all technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

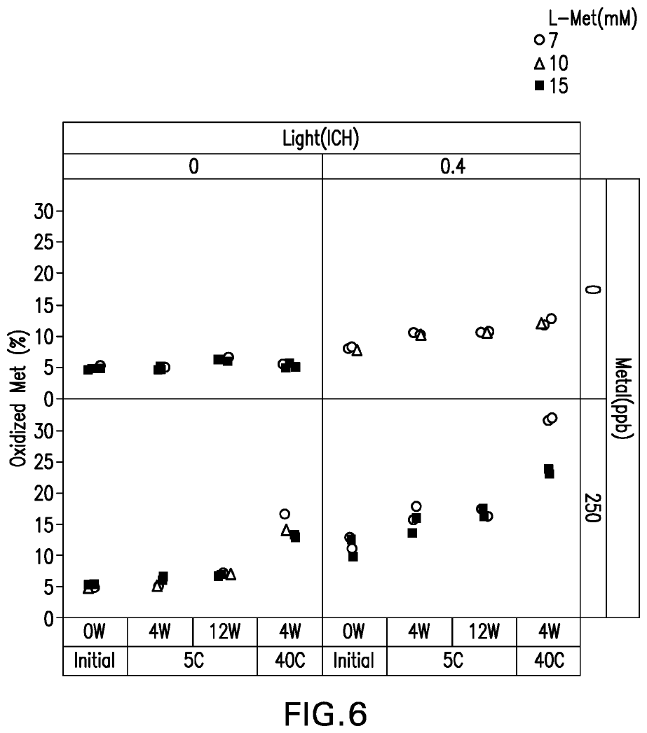

As used herein, “acidic variant” refers to the anti-ILT4 antibody that is more acidic (e.g., as determined by cation exchange chromatography) than the anti-ILT4 antibody main species. Such acidic variations are detected by various chromatography purification methods for separating molecule variants by charge, such as ion exchange, for example, cation exchange chromatography or WCX-10 HPLC (a weak cation exchange chromatography), optionally followed by mass spectroscopy. Generally, the acidic variant has a lower isoelectric point (pI) than the main species, and can have a more acidic character due to for example, methionine oxidation, sialylation of asparagine residues or deamidated variants of the antibody, or a combination thereof. In one embodiment, the anti-ILT4 antibody acidic variants are the anti-ILT4 antibody species as identified by the acidic variants peak(s) in FIG. 6D, and eluted according to the cation ion exchange method described in Example 11. In an ion exchange chromatography method, the “% acidic variants species” refers to the total area of acidic variants peak(s) divided by the total area of all peaks in the elution chromatogram. In one embodiment, the anti-ILT4 antibody acidic variant is as identified by peak(s) eluted prior to the main peak according to a cation ion exchange method. In another embodiment, the anti-ILT4 antibody acidic variant is as identified by peak(s) eluted prior to the main peak according to a weak cation ion exchange method. In an ion exchange chromatography method, the “% acidic variant” refers to the total area of acidic species peaks divided by the total area of all peaks in the elution chromatogram.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including KinExA and Biacore.

As used herein, the term “antibody” includes, but is not limited to, monoclonal antibodies, polyclonal antibodies, multispecific antibodies (e.g., bispecific antibodies), fully human antibodies, and chimeric antibodies.

As used herein, unless otherwise indicated. “antigen-binding fragment” refers to antigen-binding fragments of antibodies, i.e., antibody fragments that retain the ability to bind to the antigen bound by the full-length antibody, e.g., fragments that retain one or more CDR regions. Examples of antibody binding fragments include, but are not limited to, Fab, Fab′, F(ab′)₂, Fv fragments and individual antibody heavy chains or light chains, and individual heavy chain or light chain variable regions.

As used herein, “basic variant” refers to the anti-ILT4 antibody that is more basic (e.g., as determined by cation exchange chromatography) than the anti-ILT4 antibody main species. Such basic variants are detected by various chromatography purification methods for separating molecule variants by charge, such as ion exchange, for example, cation exchange chromatography (e.g., the method described in Example 11 or WCX-10 HPLC (a weak cation exchange chromatography), optionally followed by mass spectroscopy. Generally, the basic species has a higher pH than the main species, and can have a more basic character due to modifications or differences from the main species. In one embodiment, the anti-ILT4 antibody basic species is as identified by peak(s) eluted after the main peak according to a cation ion exchange method. In another embodiment, the anti-ILT4 antibody basic species is as identified by peak(s) eluted after the main peak according to a weak cation ion exchange method. In an ion exchange chromatography method, the “% basic species” refers to the total area of basic species peak(s) divided by the total area of all peaks in the elution chromatogram.

A “Fab fragment” is comprised of one light chain and the CH1 and variable regions of one heavy chain. The heavy chain of a Fab molecule cannot form a disulfide bond with another heavy chain molecule. An “Fab fragment” can be the product of papain cleavage of an antibody.

An “Fc” region contains two heavy chain fragments comprising the CH1 and CH2 domains of an antibody. The two heavy chain fragments are held together by two or more disulfide bonds and by hydrophobic interactions of the CH3 domains.

A “Fab′ fragment” contains one light chain and a portion or fragment of one heavy chain that contains the V_{H} domain and the CH1 domain and also the region between the CH1 and CH2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains. An “F(ab′)2 fragment” can be the product of pepsin cleavage of an antibody.

The “Fv region” comprises the variable regions from both the heavy and light chains, but lacks the constant regions.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

As used herein, “main species” or “main” refers to the anti-ILT4 antibody species identified as the majority of the antibody species in a mixture with one or more acidic or basic species thereof. Such main species are detected by various chromatography purification methods for separating molecule variants by charge, such as ion exchange, for example, cation exchange chromatography (e.g., the method described in Example 11) or WCX-10 HPLC (a weak cation exchange chromatography), optionally followed by mass spectroscopy. The mixture can be a result of for example, antibody preparations from mammalian cells and post-translational modifications thereof, upstream and downstream processing, or storage. In one embodiment, the main species is identified as the main peak according to a cation ion exchange method. In an ion exchange method, the “% main” refers to the total area of main peak divided by the total area of all peaks in the elution chromatogram. In one aspect of measuring the main species, acidic species or basic species, a Thermo Scientific ProPac WCX-10 column is used for the cation ion exchange method. In another embodiment, a Thermo Scientific ProPac WCX-10 column is used, with a Mobile Phase (A) 24 mM MES pH 6.1 with 4% acetonitrile, and mobile phase (B) 20 mM sodium phosphate, 95 mM NaCl pH 8.0 with 4% acetonitrile, and a column temperature of 35° C. In one embodiment, a non-linear gradient is used with: 22%-22% B for 0-0.6 min; 22%-29% B for 0.6-15.0 min; 29%-70% B for 15.0-30.0 min; 70%-100% B for 30.0-30.5 min; and 100%-100% B from 30.5-33.0 min. In a further embodiment, the cation ion exchange method is described in Example 11.

The term “monoclonal antibody”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

The term “fully human antibody” or “human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic “antibody” structural unit comprises a tetramer. In a monospecific antibody, each tetramer includes two identical pairs of polypeptide chains, each pair having one “light” (about 25 kDa) and one “heavy” chain (about 50-70 kDa). The amino-terminal portion of each chain includes a “variable region” or “variable domain” of about 100 to 110 or more amino acids primarily responsible for antigen recognition. The carboxy-terminal portion of the heavy chain may define a constant region primarily responsible for effector function.

Typically, human constant light chains are classified as kappa and lambda light chains. Furthermore, human constant heavy chains are typically classified as mu, delta, gamma, alpha, or epsilon, and define the antibody's isotype as IgM, IgD, IgG, IgA, and IgE, respectively. Subtypes of these IgG include, for example, IgG1 and IgG4. The invention includes anti-ILT4 antibodies and antigen-binding fragments comprising any of these light and/or heavy constant chains.

“Variable region,” variable domain, ““V region,” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75: Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved β-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | IMGT | Kabat | AbM | Chothia | Contact |
|---|---|---|---|---|---|
| V_{H} CDR1 | 27-38 | 31-35 | 26-35 | 26-32 | 30-35 |
| V_{H} CDR2 | 56-65 | 50-65 | 50-58 | 52-56 | 47-58 |
| V_{H} CDR3 | 105-117 | 95-102 | 95-102 | 95-102 | 93-101 |
| V_{L} CDR1 | 27-38 | 24-34 | 24-34 | 24-34 | 30-36 |
| V_{L} CDR2 | 56-65 | 50-56 | 50-56 | 50-56 | 46-55 |
| V_{L} CDR3 | 105-117 | 89-97 | 89-97 | 89-97 | 89-96 |

Sequence identity refers to the degree to which the amino acids of two polypeptides are the same at equivalent positions when the two sequences are optimally aligned.

Sequence similarity includes identical residues and non-identical, biochemically related amino acids. Biochemically related amino acids that share similar properties and may be interchangeable are discussed above.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of apolypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “epitope,” as used herein, refers to an area or region on an antigen to which an antibody or antigen-binding fragment binds. Binding of an antibody or antigen-binding fragment thereof disclosed herein to an epitope means that the antibody or antigen-binding fragment thereof binds to one or more amino acid residues within the epitope.

“Treat” or “treating” a cancer or an infectious condition as used herein means to administer a formulation of the invention to a subject having an immune condition or cancerous condition, or diagnosed with a cancer or pathogenic infection (e.g., viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, inhibiting the growth or survival of tumor cells, eliminating or reducing the size of one or more cancerous lesions or tumors, decreasing the level of one or more tumor markers, ameliorating, reducing the severity or duration of cancer, prolonging the survival of a patient relative to the expected survival in a similar untreated patient.

The term “cancer”, “cancerous”, or “malignant” refers to or describes the physiological condition in mammals that is typically characterized by unregulated cell growth. Examples of cancer include, but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the patient is an adult patient. In other embodiments, the patient is a pediatric patient. A patient “in need of treatment” is an individual diagnosed with, suspected of having, or predisposed to a disease or disorder in which a formulation of the invention is intended to treat or ameliorate (e.g., an ILT4-related disease such as cancer), or a patient for whom prevention of such a disorder is desired.

The term “therapeutically effective amount” or “effective amount” means an amount whereby sufficient therapeutic composition or formulation is introduced to a patient to treat a diseased or condition. One skilled in the art recognizes that this level may vary according to the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling, and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation oft 0.1%, 0.5%, 1%, 2%, 3%, 4%, 5%, or 10%.

As used herein, “x % (w/v)” is equivalent to x g/100 ml (for example 5% w/v equals 50 mg/ml).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise” or “comprises” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

The term “buffer” encompasses those agents which maintain the solution pH of the formulations in an acceptable range, or, for lyophilized formulations of the invention, provide an acceptable solution pH prior to lyophilization.

The terms “lyophilization,” “lyophilized,” and “freeze-dried” refer to a process by which the material to be dried is first frozen and then the ice or frozen solvent is removed by sublimation in a vacuum environment. Excipients may be included in pre-lyophilized formulations to enhance stability of the lyophilized product upon storage.

The term “pharmaceutical formulation” refers to preparations which are in such a form as to permit the active ingredients to be effective, and which contains no additional components which are toxic to the subjects to which the formulation would be administered. The term “formulation” and “pharmaceutical formulation” are used interchangeably throughout.

“Pharmaceutically acceptable” refers to excipients (vehicles, additives) and compositions that can reasonably be administered to a subject to provide an effective dose of the active ingredient employed and that are “generally regarded as safe,” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

A “reconstituted” formulation is one that has been prepared by dissolving a lyophilized protein formulation in a diluent such that the protein is dispersed in the reconstituted formulation. The reconstituted formulation is suitable for administration, e.g., parenteral administration), and may optionally be suitable for subcutaneous administration.

A “stable” formulation is one in which the protein therein essentially retains its physical stability and/or chemical stability and/or biological activity upon storage or under stress conditions. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 6 months. In one embodiment, a stable formulation is a formulation with no significant changes observed at a refrigerated temperature (2-8° C.) for at least 12 months. In another embodiment, a stable formulation is a formulation with no significant changes observed at a refrigerated temperature (2-8° C.) for at least 24 months. In another embodiment, stable formulation is a formulation with no significant changes observed at room temperature (23-27° C.) for at least 3 months. In another embodiment, stable formulation is a formulation with no significant changes observed at room temperature (23-27° C.) for at least 6 months. In another embodiment, stable formulation is a formulation with no significant changes observed at room temperature (23-27° C.) for at least 12 months. In another embodiment, stable formulation is a formulation with no significant changes observed at room temperature (23-27° C.) for at least 18 months. The criteria for stability for an antibody formulation are as follows. Typically, no more than 10%, preferably 5%, of antibody monomer is degraded as measured by SEC-HPLC. Typically, the formulation is colorless, or clear to slightly opalescent by visual analysis. Typically, the concentration, pH, and osmolality of the formulation have no more than +/−10% change. Potency is typically within 60-140%, preferably 80-120% of the control or reference. Typically, no more than 10%, preferably 5% of clipping of the antibody is observed, i.e., % low molecular weight species as determined, for example, by HP-SEC. Typically, no more than 10%, preferably no more than 5% of aggregation of the antibody is observed, i.e., % high molecular weight species as determined, for example, by HP-SEC.

An antibody “retains its physical stability” in a pharmaceutical formulation, if it shows no significant increase of aggregation, precipitation, and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

### Anti-ILT4 Antibodies and Antigen-Binding Fragments Thereof

The formulations disclosed herein can be used with any antibodies and antigen-binding fragments thereof (e.g., fully human antibodies) that bind to ILT4. In one embodiment, the anti-ILT4 antibody or antigen-binding fragment thereof is an antagonist. In another embodiment, the anti-ILT4 antibody or antigen-binding fragment thereof is an anti-ILT4 antibody or antigen-binding fragment thereof described herein (e.g., 1E1, 2A6, 3G7, or 2C1).

In one aspect, the formulations can be used with anti-ILT4 antibodies and antigen-binding fragments thereof as set forth herein having one or more of the properties set forth below:
binds human ILT4 at one or more amino acid residues in LYREKKSASW (SEQ ID NO:59), TRIRPEL (SEQ ID NO:60), NGQF (SEQ ID NO:61), and/or HTGRYGCQ (SEQ ID NO:62), and/or protects LYREKKSASW (SEQ ID NO:59), TRIRPEL (SEQ ID NO:60), NGQF (SEQ ID NO:61), and/or HTGRYGCQ (SEQ ID NO:62) from deuterium (e.g., DO) exchange, e.g., as determined by hydrogen-deuterium exchange mass spectrometry;
binds human ILT4 at domain 1 (see Wilcox et al. BMC Structural Biology 2:6 (2002));
binds human ILT4 extracellular domain or TM form of ILT4 expressed on a cell surface, e.g., a pre-B cell, Chinese hamster ovary cell, U937 cell, or Jurkat JE6 cell.
calculated pI ˜7.29 (e.g., 7.29 or 7.30);
experimentally determined pI ˜7.2;
is characterized by a thermogram having Tm onset >60° C., Tm1 ˜65.2° C. and Tm2 ˜78.8° C.;
binds human ILT4 with a K_{D} of about 1.7×10⁻⁸ M (e.g., as determined by surface plasmon resonance, e.g., binding of anti-ILT4 to polyhistidine tagged human ILT4);
Ka=5.5×10⁻⁵ M⁻¹ s⁻¹ (e.g., as determined by surface plasmon resonance, e.g., binding of anti-ILT4 to polyhistidine tagged human ILT4);
Kd=9×10⁻³ s⁻¹ (e.g., as determined by surface plasmon resonance, e.g., binding of anti-ILT4 to polyhistidine tagged human ILT4);
blocks binding of HLA-G (e.g., Fc fused HLA-G) to human ILT4 (e.g., ILT4 on mouse 3A9 T cells transfected with and expressing ILT4), e.g., with an ICso of about 0.25 micrograms/ml (±0.06 micrograms/ml), e.g., as determined by surface plasmon resonance;
blocks binding of HLA-A, HLA-B (e.g., fluorochrome labeled dexamers of HLA-A, such as HLA*A2:01 or HLA-B such as HLA*B7:02), and/or HLA-F (e.g., fluorochrome labeled tetramers of HLA-F) to ILT4 (e.g., ILT4 on mouse 3A9 T cells transfected with and expressing ILT4), e.g., as determined by surface plasmon resonance;
blocks ILT4 (e.g., ILT4 on mouse 3A9 T cells transfected with and expressing ILT4), binding to ANGPTL1, ANGPTL4, and/or ANGPTL7 (e.g., biotinylated ANGPTL proteins), e.g., as determined by surface plasmon resonance;
does not bind to ILT2, ILT3, ILT5, LILRB5, LILRA1, LILRA2, ILT7, ILT8, and/or ILT11;
reverses ILT4-mediated suppression of IL2 in ILT4 transfected 3A9 cells, e.g., with an EC₅₀ of 0.43 micrograms/ml (+0.14 micrograms/ml);
rescues ILT4:HLA-G induced suppression of mast cell degranulation (e.g., In the presence of plate-bound HLA-G tetramer), for example, wherein the mast cells express ILT4 and CD200RLa and are stimulated, for example, with antibody-mediated cross-linking of CD200RLa;
enhances lipopolysaccharide (LPS)-induced expression of proinflammatory myeloid cytokines, for example, GM-CSF and/or TNFalpha, from a peripheral blood mononuclear cell (PBMC);
enhances anti-CD3-induced expression of pro-inflammatory myeloid cytokines for example, GM-CSF and/or TNFalpha, from a peripheral blood mononuclear cell (PBMC);
inhibits tumor growth in humans or, for example, in other mammals such as mice (e.g., Immuno-deficient NSG mice) which were reconstituted with human hematopoietic stem cells, for example, which harbor peripheral human CD45+ immune cells, for example, wherein the tumor is a human skin melanoma tumor such as from the cell line SKMEL5;
relieves MDSC-mediated tumor tolerance in the body of a subject (e.g., human subject) with a tumor;
does not bind to cynomolgus monkey ILT4 and/or mouse pirB;
stains CD14+ human monocytes and/or CD11B+ human granulocytes; and/or
binds to one or more (e.g., 1, 2, 3, 4, 5, 6, 7, 8, 9 or all 10) of the human ILT4 haplotypes.

Antibody 1E1 (Q1E) heavy chain (IgG4)
Heavy chain
EVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCARLPTRWVTTRYFDLWGRGTLVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLGG
PSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 1; variable domain underscored; CDRs double underscored)
Heavy chain variable domain
(SEQ ID NO: 63)
EVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCARLPTRWVTTRYFDLWGRGTLVT
VSS
Antibody 1E1 (Q1E, S54A) heavy chain (IgG4)
Heavy chain
EVQLQQWGAGLLKPSETLSLTCAVYGGSFSGYYWSWIRQPPGKGLEWIGEINHAGSTNY
NPSLKSRVTISVDTSKNQFSLKLSSVTAADTAVYYCARLPTRWVTTRYFDLWGRGTLVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLGG
PSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 2; variable domain underscored; CDRs double underscored)
Heavy chain variable domain
(SEQ ID NO: 57)
EVQLQQWGAGLLKPSETLSLTCAVYGGSFSGYYWSWIRQPPGKGLEWIGEINHAGSTNY
NPSLKSRVTISVDTSKNQFSLKLSSVTAADTAVYYCARLPTRWVTTRYFDLWGRGTLVT
VSS
Antibody 1E1 heavy chain (IgG1)
Heavy chain
QVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCARLPTRWVTTRYFDLWGRGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
(SEQ ID NO: 44; variable domain underscored; CDRs double underscored)
Heavy chain variable domain
(SEQ ID NO: 69)
QVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCARLPTRWVTTRYFDLWGRGTLVT
VSS
1E1 heavy chain CDRs
CDR-H1:
(SEQ ID NO: 16)
GYYWS
CDR-H2:
(SEQ ID NO: 17)
EINHXGSTNYNPSLKS wherein X is S or A (e.g., EINHSGSTNYNPSLKS (SEQ ID
NO: 47) or EINHAGSTNYNPSLKS(SEQ ID NO: 48))
CDR-H3:
(SEQ ID NO: 18)
LPTRWVTTRYFDL
Antibody 1E1 (Q1E) light chain (lambda)
Light chain
ESVLTQPPSVSGAPGQRVTISCTGSSSNIGAGYDVHWYQQLPGTAPKLLIYGNSNRPSGV
PDRFSVSKSGASASLAITGLQAEDEADYYCQSFDNSLSAYVFGGGTQLTVLGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
(SEQ ID NO: 3; variable domain underscored; CDRs double underscored)
Light chain variable domain
(SEQ ID NO: 70)
ESVLTQPPSVSGAPGQRVTISCTGSSSNIGAGYDVHWYQQLPGTAPKLLIYGNSNRPSGV
PDRFSVSKSGASASLAITGLQAEDEADYYCQSFDNSLSAYVFGGGTQLTVL
Antibody 1E1 (Q1E, S54A) light chain (lambda)
Light chain
ESVLTQPPSVSGAPGQRVTISCTGSSSNIGAGYDVHWYQQLPGTAPKLLIYGNANRPSGV
PDRFSVSKSGASASLAITGLQAEDEADYYCQSFDNSLSAYVFGGGTQLTVLGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
(SEQ ID NO: 4; variable domain underscored; CDRs double underscored)
Light chain variable domain
(SEQ ID NO: 71)
ESVLTQPPSVSGAPGQRVTISCTGSSSNIGAGYDVHWYQQLPGTAPKLLIYGNANRPSGV
PDRFSVSKSGASASLAITGLQAEDEADYYCQSFDNSLSAYVFGGGTQLTVL
Antibody 1E1 (Q1E, N53Q) light chain (lambda)
Light chain
ESVLTQPPSVSGAPGQRVTISCTGSSSNIGAGYDVHWYQQLPGTAPKLLIYGQSNRPSGV
PDRFSVSKSGASASLAITGLQAEDEADYYCQSFDNSLSAYVFGGGTQLTVLGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
(SEQ ID NO: 5; variable domain underscored; CDRs double underscored)
Light chain variable domain
(SEQ ID NO: 72)
ESVLTQPPSVSGAPGQRVTISCTGSSSNIGAGYDVHWYQQLPGTAPKLLIYGQSNRPSGV
PDRFSVSKSGASASLAITGLQAEDEADYYCQSFDNSLSAYVFGGGTQLTVL
Antibody 1E1 (Q1E, N53E) light chain (lambda)
Light chain
ESVLTQPPSVSGAPGQRVTISCTGSSSNIGAGYDVHWYQQLPGTAPKLLIYGESNRPSGV
PDRFSVSKSGASASLAITGLQAEDEADYYCQSFDNSLSAYVFGGGTQLTVLGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
(SEQ ID NO: 6; variable domain underscored; CDRs double underscored)
Light chain variable domain
(SEQ ID NO: 73)
ESVLTQPPSVSGAPGQRVTISCTGSSSNIGAGYDVHWYQQLPGTAPKLLIYGESNRPSGV
PDRFSVSKSGASASLAITGLQAEDEADYYCQSFDNSLSAYVFGGGTQLTVL
Antibody 1E1 (Q1E, N53D) light chain (lambda)
Light chain
ESVLTQPPSVSGAPGQRVTISCTGSSSNIGAGYDVHWYQQLPGTAPKLLIYGDSNRPSGV
PDRFSVSKSGASASLAITGLQAEDEADYYCQSFDNSLSAYVFGGGTQLTVLGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
(SEQ ID NO: 7; variable domain underscored; CDRs double underscored)
Light chain variable domain
(SEQ ID NO: 58)
ESVLTQPPSVSGAPGQRVTISCTGSSSNIGAGYDVHWYQQLPGTAPKLLIYGDSNRPSGV
PDRFSVSKSGASASLAITGLQAEDEADYYCQSFDNSLSAYVFGGGTQLTVL
Antibody 1E1 light chain (lambda)
Light chain
QSVLTQPPSVSGAPGQRVTISCTGSSSNIGAGYDVHWYQQLPGTAPKLLIYGNSNRPSGV
PDRFSVSKSGASASLAITGLQAEDEADYYCQSFDNSLSAYVFGGGTQLTVLGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
(SEQ ID NO: 45; variable domain underscored; CDRs double underscored)
Light chain variable domain
(SEQ ID NO: 77)
QSVLTQPPSVSGAPGQRVTISCTGSSSNIGAGYDVHWYQQLPGTAPKLLIYGNSNRPSGV
PDRFSVSKSGASASLAITGLQAEDEADYYCQSFDNSLSAYVFGGGTQLTVL
1E1 light chain CDRs
CDR-L1:
(SEQ ID NO: 19)
TGSSSNIGAGYDVH
CDR-L2:
(SEQ ID NO: 20)
GX₁X₂NRPS; wherein X₁ is N, Q, E or D and X₂ is S or A
(e.g., GNSNRPS (SEQ ID NO: 49), GQSNRPS (SEQ ID NO: 50), GESNRPS (SEQ ID
NO: 51), GDSNRPS (SEQ ID NO: 52), GNANRPS (SEQ ID NO: 53), GQANRPS (SEQ ID
NO: 54), GEANRPS (SEQ ID NO: 55), or GDANRPS (SEQ ID NO: 56))
CDR-L3:
(SEQ ID NO: 21)
QSFDNSLSAYV

Antibodies and antigen-binding fragments thereof including the 1E1 heavy and light chain CDRs or the 1E1 V_{H} and V_{L} or the 1E1 heavy chain and light chain (or a variant thereof, e.g., as set forth herein) may be referred to as “1E1.”

Antibody 2A6 (Q1E) heavy chain (IgG4)
Heavy chain
EVQLVQSGAEVKKPGSSVKVSCKASGGTFSSYAISWVRQAPGQGLEWMGGIIPIFGTAN
YAQKFQGRVTITADESTSTAYMELSSLRSEDTAVYYCARYFDSSGWYKGGAFDIWGQG
TMVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
(SEQ ID NO: 8; variable domain underscored; CDRs double underscored)
Heavy chain variable domain
(SEQ ID NO: 64)
EVQLVQSGAEVKKPGSSVKVSCKASGGTFSSYAISWVRQAPGQGLEWMGGIIPIFGTAN
YAQKFQGRVTITADESTSTAYMELSSLRSEDTAVYYCARYFDSSGWYKGGAFDIWGQG
TMVTVSS
Antibody 2A6 (Q1E, S102A, M119L) heavy chain (IgG4)
Heavy chain
EVQLVQSGAEVKKPGSSVKVSCKASGGTFSSYAISWVRQAPGQGLEWMGGIIPIFGTAN
YAQKFQGRVTITADESTSTAYMELSSLRSEDTAVYYCARYFDASGWYKGGAFDIWGQG
TLVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
(SEQ ID NO: 9; variable domain underscored; CDRs double underscored)
Heavy chain variable domain
(SEQ ID NO: 65)
EVQLVQSGAEVKKPGSSVKVSCKASGGTFSSYAISWVRQAPGQGLEWMGGIIPIFGTAN
YAQKFQGRVTITADESTSTAYMELSSLRSEDTAVYYCARYFDASGWYKGGAFDIWGQG
TLVTVSS
Antibody 2A6 (Q1E, D101S, M119L) heavy chain (IgG4)
Heavy chain
EVQLVQSGAEVKKPGSSVKVSCKASGGTFSSYAISWVRQAPGQGLEWMGGIIPIFGTAN
YAQKFQGRVTITADESTSTAYMELSSLRSEDTAVYYCARYFSSSGWYKGGAFDIWGQG
TLVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
(SEQ ID NO: 10; variable domain underscored; CDRs double underscored)
Heavy chain variable domain
(SEQ ID NO: 66)
EVQLVQSGAEVKKPGSSVKVSCKASGGTFSSYAISWVRQAPGQGLEWMGGIIPIFGTAN
YAQKFQGRVTITADESTSTAYMELSSLRSEDTAVYYCARYFSSSGWYKGGAFDIWGQG
TLVTVSS

The formulations disclosed herein can be used with antibodies comprising SEQ ID NO:8, 9, 10, 64, 65, or 66, and antigen-binding fragments thereof, wherein residue 1 of SEQ ID NO:8, 9, 10, 64, 65, or 66 is Q instead of E.

2A6 heavy chain CDRs
CDR-H1:
(SEQ ID NO: 22)
SYAIS
CDR-H2:
(SEQ ID NO: 23)
GIIPIFGTANYAQKFQG
CDR-H3:
(SEQ ID NO: 24)
YFX₁X₂SGWYKGGAFDI; wherein X₁ is D or S and X₂ is S or A
(e.g., YFDSSGWYKGGAFDI (SEQ ID NO: 91), YFSSSGWYKGGAFDI (SEQ ID
NO: 92), YFDASGWYKGGAFDI (SEQ ID NO: 93), or YFSASGWYKGGAFDI (SEQ ID
NO: 94))
Antibody 2A6 light chain (lambda)
Light chain
QSVLTQPSSLSASPGASASLTCTLRSGINVDTYRIHWYQQKPGSPPQYLLRYKSDSDKHQ
GSGVPSRFSGSKDPSANAGILLISGLQSEDEADYYCAIWYSSTWVFGGGTQLTVLGQPKA
APSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECS
(SEQ ID NO: 11; variable domain underscored; CDRs double underscored)
Light chain variable domain
(SEQ ID NO: 74)
QSVLTQPSSLSASPGASASLTCTLRSGINVDTYRIHWYQQKPGSPPQYLLRYKSDSDKHQ
GSGVPSRFSGSKDPSANAGILLISGLQSEDEADYYCAIWYSSTWVFGGGTQLTVL

The formulations disclosed herein can be used with antibodies comprising SEQ ID NO:11 or 74 and antigen-binding fragments thereof, wherein residue 1 of SEQ ID NO:11 or 74 is E instead of Q.

2A6 light
chain CDRs
CDR-L1:
(SEQ ID NO: 5)
TLRSGINVDTYRIH
CDR-L2:
(SEQ ID NO: 26)
YKSDSDKHQGS
CDR-L3:
(SEQ ID NO: 27)
AIWYSSTWV

Antibodies and antigen-binding fragments thereof including the 2A6 heavy and light chain CDRs or the 2A6 V_{H} and V_{L} or the 2A6 heavy chain and light chain (or a variant thereof, e.g., as set forth herein) may be referred to as “2A6.”

Antibody 3G7 (Q1E) heavy chain (IgG4)
Heavy chain
EVQLVESGGGVVQPGRSLRLSCAASGFTFSSYAMHWVRQAPGKGLEWVAVISYDGSNK
YYADSVKGRFTISRDNSKNTLYLQMNSLRAEDTAVYYCARVGEWIQLWSPFDYWGQG
TLVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
(SEQ ID NO: 12; variable domain underscored; CDRs double underscored).
Heavy chain variable domain
(SEQ ID NO: 67)
EVQLVESGGGVVQPGRSLRLSCAASGFTFSSYAMHWVRQAPGKGLEWVAVISYDGSNK
YYADSVKGRFTISRDNSKNTLYLQMNSLRAEDTAVYYCARVGEWIQLWSPFDYWGQG
TLVTVSS

The formulations disclosed herein can be used with antibodies comprising SEQ ID NO:12 or 67 and antigen-binding fragments thereof wherein residue 1 of SEQ ID NO:12 or 67 is Q instead of E.

3G7 heavy chain CDRs
CDR-H1:
(SEQ ID NO: 28)
SYAMH
CDR-H2:
(SEQ ID NO: 29)
VISYDGSNKYYADSVKG
CDR-H3:
(SEQ ID NO: 30)
VGEWIQLWSPFDY
Antibody 3G7 light chain (kappa)
Light chain
DIQMTQSPSSVSASVGDRVTITCRASQGISSWLAWYQQKPGKAPKFLIYAASSLQSGVPS
KFSGSGSGTDFTLTISSLQPEDFATYYCQQYNSYPPTFGGGTKVEIKRtVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
(SEQ ID NO: 13; variable domain underscored; CDRs double underscored)
Light chain variable domain
(SEQ ID NO: 75)
DIQMTQSPSSVSASVGDRVTITCRASQGISSWLAWYQQKPGKAPKFLIYAASSLQSGVPS
KFSGSGSGTDFTLTISSLQPEDFATYYCQQYNSYPPTFGGGTKVEIK
3G7 light chain CDRs
CDR-L1:
(SEQ ID NO: 31)
RASQGISSWLA
CDR-L2:
(SEQ ID NO: 32)
AASSLQS
CDR-L3:
(SEQ ID NO: 33)
QQYNSYPPT

Antibodies and antigen-binding fragments thereof including the 3G7 heavy and light chain CDRs or the 3G7 V_{H} and V_{L} or the 3G7 heavy chain and light chain (or a variant thereof, e.g., as set forth herein) may be referred to as “3G7.”

Antibody 2C1 (Q1E) heavy chain (IgG4)
Heavy chain
EVQLVQSGAEVKKPGASVKVSCKVSGYTLTELSMHWVRQAPGKGLEWMGGFDPEDGE
TIYAQKFQGRVTMTEDTSTDTAYMELSSLRSEDTAVYYCARAGPLYTIFGVVIIPDNWFD
PWGQGTLVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK
(SEQ ID NO: 14; variable domain underscored; CDRs double underscored)
Heavy chain variable domain
(SEQ ID NO: 68)
EVQLVQSGAEVKKPGASVKVSCKVSGYTLTELSMHWVRQAPGKGLEWMGGFDPEDGE
TIYAQKFQGRVTMTEDTSTDTAYMELSSLRSEDTAVYYCARAGPLYTIFGVVIIPDNWFD
PWGQGTLVTVSS

The formulations disclosed herein can be used with antibodies comprising SEQ ID NO:14 or 68 and antigen-binding fragments thereof, wherein residue 1 of SEQ ID NO:14 or 68 is Q instead of E.

2C1 heavy chain CDRs
CDR-H1:
(SEQ ID NO: 34)
ELSMH
CDR-H2:
(SEQ ID NO: 35)
GFDPEDGETIYAQKFQG
CDR-H3:
(SEQ ID NO: 36)
AGPLYTIFGVVIIPDNWFDP
Antibody 2C1 light chain (Q1E) (lambda)
Light chain
ESVLTQPPSVSGAPGQRVTISCTGSSSNIGAGYDVHWYQQLPGTAPKLLIYGNSNRPSGV
PDRFSGSKSGTSASLAITGLQAEDEADYYCQSYDSSLSGSGVVFGGGTQLIILGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
(SEQ ID NO: 15; variable domain underscored; CDRs double underscored)
Light chain variable domain
(SEQ ID NO: 76)
ESVLTQPPSVSGAPGQRVTISCTGSSSNIGAGYDVHWYQQLPGTAPKLLIYGNSNRPSGV
PDRFSGSKSGTSASLAITGLQAEDEADYYCQSYDSSLSGSGVVFGGGTQLIIL

The formulations disclosed herein can be used with antibodies comprising SEQ ID NO:15 or 76 and antigen-binding fragments thereof, wherein residue 1 of SEQ ID NO:15 or 76 is Q instead of E.

2C1 light chain CDRs
CDR-L1:
(SEQ ID NO: 37)
TGSSSNIGAGYDVH
CDR-L2:
(SEQ ID NO: 38)
GNSNRPS
CDR-L3:
(SEQ ID NO: 39)
QSYDSSLSGSGVV

Antibodies and antigen-binding fragments thereof including the 2C1 heavy and light chain CDRs or the 2C1 V_{H} and V_{L} or the 2C1 heavy chain and light chain (or a variant thereof, e.g., as set forth herein) may be referred to as “2C1.”

In various embodiments of the antibody or antigen-binding fragment thereof, a C-terminal lysine of a heavy chain immunoglobulin is absent.

Thus, in some embodiments, the antibody or antigen-binding fragment thereof comprises a light chain immunoglobulin, a heavy chain immunoglobulin, or both a light and heavy chain immunoglobulin, wherein the light chain immunoglobulin comprises the amino acid sequence set forth in SEQ ID NO:3, 4, 5, 6, 7, 11, 13, 15, or 45; and/or the heavy chain immunoglobulin comprises the amino acid sequence set forth in SEQ ID NO: 1, 2, 8, 9, 10, 12, 14, 44, 79, 80, 81, 82, 83, 84, 85, or 86.

In certain embodiments, the antibody or antigen-binding fragment thereof comprises a heavy chain immunoglobulin comprising the amino acid sequence set forth in SEQ ID NO: 1 or 79; and a light chain immunoglobulin comprising the amino acid sequence set forth in SEQ ID NO: 3.

In some embodiments, the antibody or antigen-binding fragment thereof comprises a heavy chain immunoglobulin comprising the amino acid sequence set forth in SEQ ID NO: 2 or 80; and a light chain immunoglobulin comprising the amino acid sequence set forth in SEQ ID NO: 4.

In other embodiments, the antibody or antigen-binding fragment thereof comprises a heavy chain immunoglobulin comprising the amino acid sequence set forth in SEQ ID NO: 2 or 80; and a light chain immunoglobulin comprising the amino acid sequence set forth in SEQ ID NO: 5.

In yet other embodiments, the antibody or antigen-binding fragment thereof comprises a heavy chain immunoglobulin comprising the amino acid sequence set forth in SEQ ID NO: 2 or 80; and a light chain immunoglobulin comprising the amino acid sequence set forth in SEQ ID NO: 6.

In still other embodiments, the antibody or antigen-binding fragment thereof comprises a heavy chain immunoglobulin comprising the amino acid sequence set forth in SEQ ID NO: 2 or 80; and a light chain immunoglobulin comprising the amino acid sequence set forth in SEQ ID NO: 7.

In certain embodiments, the antibody or antigen-binding fragment thereof comprises a heavy chain immunoglobulin comprising the amino acid sequence set forth in SEQ ID NO: 2 or 80; and a light chain immunoglobulin comprising the amino acid sequence set forth in SEQ ID NO: 3.

In some embodiments, the antibody or antigen-binding fragment thereof comprises heavy chain immunoglobulin comprising the amino acid sequence set forth in SEQ ID NO: 8 or 82; and a light chain immunoglobulin comprising the amino acid sequence set forth in SEQ ID NO: 11.

In other embodiments, the antibody or antigen-binding fragment thereof comprises a heavy chain immunoglobulin comprising the amino acid sequence set forth in SEQ ID NO: 9 or 83; and a light chain immunoglobulin comprising the amino acid sequence set forth in SEQ ID NO: 11.

In yet other embodiments, the antibody or antigen-binding fragment thereof comprises a heavy chain immunoglobulin comprising the amino acid sequence set forth in SEQ ID NO: 10 or 84; and a light chain immunoglobulin comprising the amino acid sequence set forth in SEQ ID NO: 11.

In still other embodiments, the antibody or antigen-binding fragment thereof comprises a heavy chain immunoglobulin comprising the amino acid sequence set forth in SEQ ID NO: 12 or 85; and a light chain immunoglobulin comprising the amino acid sequence set forth in SEQ ID NO: 13.

In yet still embodiments, the antibody or antigen-binding fragment thereof comprises a heavy chain immunoglobulin comprising the amino acid sequence set forth in SEQ ID NO: 14 or 86; and a light chain immunoglobulin comprising the amino acid sequence set forth in SEQ ID NO: 15.

In certain other embodiments, the antibody or antigen-binding fragment thereof comprises a light chain immunoglobulin, a heavy chain immunoglobulin, or both a light and heavy chain immunoglobulin, wherein the light chain variable domain comprises the amino acid sequence set forth in SEQ ID NO:70, 71, 72, 73, 58, 74, 75, 76, or 77, and/or the heavy chain variable domain comprises the amino acid sequence set forth in SEQ ID NO:63, 57, 64, 65, 66, 67, 68, or 69.

In certain embodiments, the antibody or antigen-binding fragment thereof comprises a heavy chain variable domain comprising the amino acid sequence set forth in SEQ ID NO:63; and a light chain variable domain comprising the amino acid sequence set forth in SEQ ID NO:70.

In some embodiments, the antibody or antigen-binding fragment thereof comprises a heavy chain variable domain comprising the amino acid sequence set forth in SEQ ID NO:57; and a light chain variable domain comprising the amino acid sequence set forth in SEQ ID NO:71.

In other embodiments, the antibody or antigen-binding fragment thereof comprises a heavy chain variable domain comprising the amino acid sequence set forth in SEQ ID NO:57; and a light chain variable domain comprising the amino acid sequence set forth in SEQ ID NO:72.

In yet other embodiments, the antibody or antigen-binding fragment thereof comprises a heavy chain variable domain comprising the amino acid sequence set forth in SEQ ID NO:57; and a light chain variable domain comprising the amino acid sequence set forth in SEQ ID NO:73.

In still other embodiments, the antibody or antigen-binding fragment thereof comprises a heavy chain variable domain comprising the amino acid sequence set forth in SEQ ID NO:57; and a light chain variable domain comprising the amino acid sequence set forth in SEQ ID NO:58.

In certain embodiments, the antibody or antigen-binding fragment thereof comprises a heavy chain variable domain comprising the amino acid sequence set forth in SEQ ID NO:57; and a light chain variable domain comprising the amino acid sequence set forth in SEQ ID NO:70.

In some embodiments, the antibody or antigen-binding fragment thereof comprises a heavy chain variable domain comprising the amino acid sequence set forth in SEQ ID NO:64; and a light chain variable domain comprising the amino acid sequence set forth in SEQ ID NO:74.

In other embodiments, the antibody or antigen-binding fragment thereof comprises a heavy chain variable domain comprising the amino acid sequence set forth in SEQ ID NO:65; and a light chain variable domain comprising the amino acid sequence set forth in SEQ ID NO:74.

In yet other embodiments, the antibody or antigen-binding fragment thereof comprises a heavy chain variable domain comprising the amino acid sequence set forth in SEQ ID NO:66; and a light chain variable domain comprising the amino acid sequence set forth in SEQ ID NO:74.

In still other embodiments, the antibody or antigen-binding fragment thereof comprises a heavy chain variable domain comprising the amino acid sequence set forth in SEQ ID NO:67; and a light chain variable domain comprising the amino acid sequence set forth in SEQ ID NO:75.

In yet still other embodiments, the antibody or antigen-binding fragment thereof comprises a heavy chain variable domain comprising the amino acid sequence set forth in SEQ ID NO:68; and a light chain variable domain comprising the amino acid sequence set forth in SEQ ID NO:76.

In a further embodiment, the antibody or antigen-binding fragment thereof that binds ILT4 comprises an immunoglobulin light chain variable (V_{L}) domain comprising a CDR-L1, CDR-L2 and CDR-L3 of 1E1 (e.g., SEQ ID NOs: 19-21); and an immunoglobulin heavy chain variable (V_{H}) domain comprising a CDR-H1, CDR-H2 and CDR-H3 of 1E1 (e.g., SEQ ID NOs: 16-18).

In a further embodiment, the antibody or antigen-binding fragment thereof that binds ILT4 comprises an immunoglobulin light chain variable (V_{L}) domain comprising a CDR-L1, CDR-L2 and CDR-L3 of 2A6 (e.g., SEQ ID NOs: 25-27); and an immunoglobulin heavy chain variable (V_{H}) domain comprising a CDR-H1, CDR-H2 and CDR-H3 of 2A6 (e.g., SEQ ID NOs: 22-24).

In a further embodiment, the antibody or antigen-binding fragment thereof that binds ILT4 comprises an immunoglobulin light chain variable (V_{L}) domain comprising a CDR-L1, CDR-L2 and CDR-L3 of 3G7 (e.g., SEQ ID NOs: 31-33); and an immunoglobulin heavy chain variable (V_{H}) domain comprising a CDR-H1, CDR-H2 and CDR-H3 of 3G7 (e.g., SEQ ID NOs: 28-30).

In a further embodiment, the antibody or antigen-binding fragment thereof that binds ILT4 comprises an immunoglobulin light chain variable (V_{L}) domain comprising a CDR-L1, CDR-L2 and CDR-L3 of 2C1 (e.g., SEQ ID NOs: 37-39); and an immunoglobulin heavy chain variable (V_{H}) domain comprising a CDR-H1, CDR-H2 and CDR-H3 of 2C1 (e.g., SEQ ID NOs: 34-36).

In one embodiment, the antibody or antigen-binding fragment comprises a V_{H} domain comprising CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising CDR-L1: TGSSSNIGAGYDVH (SEQ ID NO: 19), CDR-L2: GNSNRPS (SEQ ID NO: 49), and CDR-L3: QSFDNSLSAYV (SEQ ID NO: 21).

In another embodiment, the antibody or antigen-binding fragment comprises a V_{H} domain comprising: CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GQSNRPS (SEQ ID NO: 50), and CDR-L3: QSFDNSLSAYV (SEQ ID NO: 21).

In yet another embodiment, the antibody or antigen-binding fragment thereof comprises a V_{H} domain comprising: CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GESNRPS (SEQ ID NO: 51), and CDR-L3: QSFDNSLSAYV (SEQ ID NO: 21).

In still another embodiment, the antibody or antigen-binding fragment thereof comprises: a V_{H} domain comprising: CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GDSNRPS (SEQ ID NO: 52), and CDR-L3: QSFDNSLSAYV (SEQ ID NO: 21).

In one embodiment, the antibody or antigen-binding fragment thereof comprises: a V_{H} domain comprising: CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GNANRPS (SEQ ID NO: 53), and CDR-L3: QSFDNSLSAYV (SEQ ID NO: 21).

In another embodiment, the antibody or antigen-binding fragment thereof comprises: a V_{H} domain comprising: CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GQANRPS (SEQ ID NO: 54), and CDR-L3: QSFDNSLSAYV (SEQ ID NO: 21).

In yet another embodiment, the antibody or antigen-binding fragment thereof comprises: a V_{H} domain comprising: CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GEANRPS (SEQ ID NO: 55), and CDR-L3: QSFDNSLSAYV (SEQ ID NO: 21).

In still another embodiment, the antibody or antigen-binding fragment thereof comprises: a V_{H} domain comprising: CDR-H1: GYYWS (SEQ ID NO: 16), CDR-H2: EINHSGSTNYNPSLKS (SEQ ID NO: 47), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GDANRPS (SEQ ID NO: 56), and CDR-L3: QSFDNSLSAYV (SEQ ID NO: 21).

In one embodiment, the antibody or antigen-binding fragment thereof comprises: a V_{H} domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GNSNRPS (SEQ ID NO: 49), and CDR-L3: QSFDNSLSAYV (SEQ ID NO: 21).

In another embodiment, the antibody or antigen-binding fragment thereof comprises: a V_{H} domain comprising: CDR-HL: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GQSNRPS (SEQ ID NO: 50), and CDR-L3: QSFDNSLSAYV (SEQ ID NO: 21).

In yet another embodiment, the antibody or antigen-binding fragment thereof comprises: a V_{H} domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GESNRPS (SEQ ID NO: 51), and CDR-L3: QSFDNSLSAYV (SEQ ID NO: 21).

In still another embodiment, the antibody or antigen-binding fragment thereof comprises: a V_{H} domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-12: GDSNRPS (SEQ ID NO: 52), and CDR-L3: QSFDNSLSAYV (SEQ ID NO: 21).

In one embodiment, the antibody or antigen-binding fragment thereof comprises: a V_{H} domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GNANRPS (SEQ ID NO: 53), and CDR-L3: QSFDNSLSAYV (SEQ ID NO: 21).

In another embodiment, the antibody or antigen-binding fragment thereof comprises: a V_{H} domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GQANRPS (SEQ ID NO: 54), and CDR-L3: QSFDNSLSAYV (SEQ ID NO: 21).

In yet another embodiment, the antibody or antigen-binding fragment thereof comprises: a V_{H} domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GEANRPS (SEQ ID NO: 55), and CDR-L3: QSFDNSLSAYV (SEQ ID NO: 21).

In still another embodiment, the antibody or antigen-binding fragment thereof comprises: a V_{H} domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and/or, a V_{L} domain comprising: CDR-L1: TGSSSNIGAGYDVH (SEQ ID NO: 19), CDR-L2: GDANRPS (SEQ ID NO: 56), and CDR-L3: QSFDNSLSAYV (SEQ ID NO: 21).

The formulations disclosed herein can be used with an antibody or antigen-binding fragment thereof that binds ILT4 and comprises the V_{L} domain of antibody 1E1 (e.g., SEQ ID NO:70, 71, 72, 73, 58 or 77) and/or the V_{H} domain of antibody 1E1 (e.g., SEQ ID NO:63, 57 or 69).

The formulations disclosed herein can be used with an antibody or antigen-binding fragment thereof that binds ILT4 and comprises the V_{L} domain of antibody 2A6 (e.g., SEQ ID NO:74) and/or the V_{H} domain of antibody 2A6 (e.g., SEQ ID NO:64, 65 or 66).

The formulations disclosed herein can be used with an antibody or antigen-binding fragment thereof that binds ILT4 and comprises the V_{L} domain of antibody 3G7 (e.g., SEQ ID NO:75) and/or the V_{H} domain of antibody 3G7 (e.g., SEQ ID NO:67).

The formulations disclosed herein can be used with an antibody or antigen-binding fragment thereof that binds ILT4 and comprises the V_{L} domain of antibody 2C1 (e.g., SEQ ID NO:76) and/or the V_{H} domain of antibody 2C1 (e.g., SEQ ID NO:68).

The formulations disclosed herein can be used with an antibody or antigen-binding fragment thereof that binds ILT4 and comprises the light chain immumoglobulin of antibody 1E1 (e.g., SEQ ID NO:3, 4, 5, 6, 7 or 45) and/or the heavy chain immunoglobulin of antibody 1E1 (e.g., SEQ ID NO:1, 2, 44, 79, 80, or 81).

The formulations disclosed herein can be used with an antibody or antigen-binding fragment thereof that binds ILT4 and comprises the light chain immnoglobulin of antibody 2A6 (e.g., SEQ ID NO:11) and/or the heavy chain immunoglobulin of antibody 2A6 (e.g., SEQ ID NO:8, 9, 10, 82, 83, or 84).

The formulations disclosed herein can be used with an antibody or antigen-binding fragment thereof that binds ILT4 and comprises the light chain immnoglobulin of antibody 3G7 (e.g., SEQ ID NO:13) and/or the heavy chain immunoglobulin of antibody 3G7 (e.g., SEQ ID NO:12 or 85).

The formulations disclosed herein can be used with an antibody or antigen-binding fragment thereof that binds ILT4 and comprises the light chain immnoglobulin of antibody 2C1 (e.g., SEQ ID NO:15) and/or the heavy chain immunoglobulin of antibody 2C1 (e.g., SEQ ID NO:14 or 86).

The formulations disclosed herein can be used with an antibody that consists of two heavy chains and two light chains, wherein each light chain comprises the V_{L} or light chain immunoglobulin of antibody 1E1, 2A6, 3G7, or 2C1, and each heavy chain comprises the V_{H} or heavy chain immunoglobulin of antibody 1E1, 2A6, 3G7, or 2C1.

In one embodiment, the antibody consists of two heavy chains and two light chains, wherein each light chain comprises the amino acid sequence set forth in SEQ ID NO:58 and each heavy chain comprises the amino acid sequence set forth in SEQ ID NO:57.

In another embodiment, the antibody consists of two heavy chains and two light chains, wherein each light chain comprises the amino acid sequence set forth in SEQ ID NO:58 and each heavy chain comprises the amino acid sequence set forth in SEQ ID NO:57, wherein the light chain further comprises the amino acid sequence set forth in SEQ ID NO:90.

In yet another embodiment, the antibody consists of two heavy chains and two light chains, wherein each light chain comprises the amino acid sequence set forth in SEQ ID NO:58 and each heavy chain comprises the amino acid sequence set forth in SEQ ID NO:57, wherein the heavy chain further comprises the amino acid sequence set forth in SEQ ID NO:89.

In still another embodiment, the antibody consists of two heavy chains and two light chains, wherein each light chain comprises the amino acid sequence set forth in SEQ ID NO:58 and each heavy chain comprises the amino acid sequence set forth in SEQ ID NO:57, wherein the light chain further comprises the amino acid sequence set forth in SEQ ID NO:90 and the heavy chain further comprises the amino acid sequence set forth in SEQ ID NO:89.

In one embodiment, the antibody consists of two heavy chains and two light chains, wherein each light chain comprises the amino acid sequence set forth in SEQ ID NO:7 and each heavy chain comprises the amino acid sequence set forth in SEQ ID NO:2.

In another embodiment, the antibody consists of two heavy chains and two light chains, wherein each light chain consists of the amino acid sequence set forth in SEQ ID NO:7 and each heavy chain consists of the amino acid sequence set forth in SEQ ID NO:2.

In an embodiment, the antibody or antigen-binding fragment thereof comprises a V_{L} (with or without signal sequence), e.g., the V_{L} in any of SEQ ID NO:58 or 70-77, having up to 1, 2, 3, 4, 5, 6, 7, 8, 9, 10 or more conservative or non-conservative amino acid substitutions; and/or a V_{H} (with or without signal sequence), e.g., the V_{H} in any of SEQ ID NO:57 or 63-69, having up to 1, 2, 3, 4, 5, 6, 7, 8, 9, 10 or more conservative or non-conservative amino acid substitutions, while still binding to ILT4.

The formulations disclosed herein can be used with polypeptides comprising the amino acid sequences disclosed herein, e.g., SEQ ID NOs: 1-39, 44, 45, 47-58, 63-77, or 79-86, as well as polypeptides comprising such amino acid sequences with up to 1, 2, 3, 4, 5, 6, 7, 8, 9, 10, 12, 15, 20 or more conservative or non-conservative amino acid substitutions therein.

In certain embodiments, the antibody or antigen-binding fragment thereof comprises a light chain immunoglobulin, a heavy chain immunoglobulin, or both a light and heavy chain immunoglobulin, wherein the light chain immunoglobulin has at least 90% amino acid sequence identity to the amino acid sequence set forth in SEQ ID NO:3, 4, 5, 6, 7, 11, 13, 15, or 45, and/or the heavy chain immunoglobulin has at least 90% amino acid sequence identity to the amino acid sequence set forth in SEQ ID NO:1, 2, 8, 9, 10, 12, 14, 44, 79, 80, 81, 82, 83, 84, 85, or 86.

In some embodiments, the antibody or antigen-binding fragment thereof comprises a light chain immunoglobulin, a heavy chain immunoglobulin, or both a light and heavy chain immunoglobulin, wherein the light chain immunoglobulin comprises a light chain variable domain having at least 90% amino acid sequence identity to the amino acid sequence set forth in SEQ ID NO:70, 71, 72, 73, 58, 74, 75, 76, or 77, and/or the heavy chain immunoglobulin comprises a heavy chain variable domain having at least 90% amino acid sequence identity to the amino acid sequence set forth in SEQ ID NO:63, 57, 64, 65, 66, 67, 68, or 69.

In an embodiment, an immunoglobulin heavy chain of an anti-ILT4 antibody or antigen-binding fragment of the invention is operably linked to a signal sequence, e.g., comprising the amino acid sequence MEWSWVFLFFLSVTTGVHS (SEQ ID NO:41) and/or an immunoglobulin light chain of an anti-ILT4 antibody or antigen-binding fragment of the invention is operably linked to a signal sequence, e.g., comprising the amino acid sequence MSVPTQVLGLLLLWLTDARC (SEQ ID NO:42).

In an embodiment, an N-terminal glutamine (Q) of an immunoglobulin chain set forth herein (e.g., heavy and/or light) is replaced with a pyroglutamic acid. In one embodiment, an N-terminal Q of a heavy chain immunoglobulin is replaced with a pyroglutamic acid. In another embodiment, an N-terminal Q of a light chain immunoglobulin is replaced with a pyroglutamic acid. In yet another embodiment, an N-terminal Q of a heavy chain immunoglobulin and an N-terminal Q of a heavy chain immunoglobulin are replaced with a pyroglutamic acid.

The formulations disclosed herein can also be used with antibodies or antigen-binding fragments that bind to the same epitope of ILT4 (e.g., human ILT4) as any anti-ILT4 antibody or antigen-binding fragment thereof disclosed herein (e.g., 1E1, 2A6, 3G7 or 2C1). In one embodiment, the epitope is LYREKKSASW (SEQ ID NO:59). In another embodiment, the epitope is TRIRPEL (SEQ ID NO:60). In yet another embodiment, the epitope is NGQF (SEQ ID NO:61). In still another embodiment, the epitope is HTGRYGCQ (SEQ ID NO:62). In certain embodiments, the antibody or antigen-binding fragment thereof binds to the same epitope of human ILT4 as an antibody or antigen-binding fragment thereof comprising the heavy chain and light chain amino acid sequences set forth in SEQ ID NOs:1 and 3; 2 and 4; 2 and 5; 2 and 6; 2 and 7; 2 and 3; 8 and 11; 9 and 11; 10 and 11; 12 and 13; 14 and 15; 79 and 3; 80 and 4; 80 and 5; 80 and 6; 80 and 7; 80 and 3; 82 and 11; 83 and 11; 84 and 11; 85 and 13; and 86 and 15; respectively. In some embodiments, the antibody or antigen-binding fragment thereof binds to the same epitope of human ILT4 as an antibody or antigen-binding fragment thereof comprising the heavy chain variable domain and light chain variable domain amino acid sequences set forth in SEQ ID NOs:63 and 70; 57 and 71; 57 and 72; 57 and 73; 57 and 58; 57 and 70; 64 and 74; 65 and 74; 66 and 74; 67 and 75; 68 and 76; respectively.

The formulations disclosed herein can be used with antibodies and antigen-binding fragments that cross-block the binding of any anti-ILT4 antibody or antigen-binding fragment thereof disclosed herein (e.g., 1E1, 2A6, 3G7 or 2C1) to ILT4 (e.g., human ILT4) or compete with any anti-ILT4 antibody or antigen-binding fragment thereof disclosed herein (e.g., IE1, 2A6, 3G7 or 2C1) to ILT4 (e.g., human ILT4). The cross-blocking antibodies and antigen-binding fragments thereof discussed herein can be identified based on their ability to block any of the antibodies or fragments specifically set forth herein from binding to ILT4, in binding assays (e.g., bio-layer interferometry (BLI; for example FORTEBIO OCTET binding assay; Pall ForteBio Corp; Menlo Park, CA), surface plasmon resonance (SPR), BIACore, ELISA, flow cytometry). For example, in an embodiment of the invention, when using BLI, the tip of a fiber-optic probe is coated with ligand (e.g., ILT4) and acts as the biosensor wherein binding of anti-ILT4 antibody or antigen-binding fragment to the ILT4 alters the interference pattern of white light reflected from the probe layer bound to ILT4 and an internal reference layer. The shift is indicative of ILT4/anti-ILT4 binding. In an embodiment of the invention, the ILT4 coated tip is immersed in a solution of analyte containing antibody or antigen-binding fragment, e.g., in the well of either a 96- or 384-well plate. In an embodiment of the invention, the plate is shaken during reading to create orbital flow. To read the assay, white light is directed down the length of the fiber. As mentioned above, interference between light reflecting off the reference layer and immobilized surfaces containing ILT4 of the tip creates a distinctive pattern of light returning up the fiber. As molecules bind to the immobilized sensor surface, that pattern changes in proportion to the extent of binding. For example, assays can be used in which an ILT4 (e.g., human ILT4) protein is immobilized on a BLI probe or plate, a reference anti-ILT4 antibody or fragment binds to ILT4 (e.g., at saturating concentration) and a test anti-ILT4 antibody or fragment is added. The ability of the test antibody to compete with the reference antibody for ILT4 binding is then determined. In the BLI format, light interference of the ILT4 complex is monitored to determine if the test antibody effectively competes with the reference antibody, e.g., nanometers of light wavelength shift over time is monitored wherein a shift indicates additional binding of the test antibody and a lack of cross-blocking. In an embodiment of the invention, in the BLI format, cross-blocking is qualitatively deemed to have occurred between the antibodies if no additional binding of test antibody is observed. In an embodiment of the invention, as a control, cross-blocking of the reference antibody with itself is confirmed; wherein the assay is determined to be operating correctly if the reference antibody can cross-block itself from ILT4 binding. The ability of a test antibody to inhibit the binding of the anti-ILT4 antibody or fragment 1E1, 2A6, 3G7 or 2C1, to ILT4 (e.g., human ILT4) demonstrates that the test antibody can cross-block the antibody or fragment for binding to ILT4 (e.g., human ILT4) and thus, may, in some cases, bind to the same epitope on ILT4 (e.g., human ILT4) as 1E1, 2A6, 3G7 and/or 2C1. As stated above, antibodies and fragments that bind to the same epitope as any of the anti-ILT4 antibodies or fragments of the invention also form part of the invention. In an embodiment of the invention, BLI is conducted in a sandwich format wherein a reference anti-ILT4 antibody or antigen-binding fragment is immobilized to the probe and then bound with ILT4. Test anti-ILT4 antibody or antigen-binding fragment is then tested for the ability to block binding of the references antibody or fragment.

In certain embodiments, the antibody or antigen-binding fragment thereof competes for binding to human ILT4 with an antibody or fragment comprising the heavy chain and light chain amino acid sequences set forth in SEQ ID NOs:1 and 3; 2 and 4; 2 and 5; 2 and 6; 2 and 7; 2 and 3; 8 and 11; 9 and 11; 10 and 11; 12 and 13; 14 and 15; 79 and 3; 80 and 4; 80 and 5; 80 and 6; 80 and 7; 80 and 3; 82 and 11; 83 and 11; 84 and 11; 85 and 13; and 86 and 15; respectively. In some embodiments, the antibody or antigen-binding fragment thereof competes for binding to human ILT4 with an antibody or fragment comprising the heavy chain variable domain and light chain variable domain amino acid sequences set forth in SEQ ID NOs:63 and 70; 57 and 71; 57 and 72; 57 and 73; 57 and 58; 57 and 70; 64 and 74; 65 and 74; 66 and 74; 67 and 75; 68 and 76; respectively.

The formulations disclosed herein can be used with anti-ILT4 antibodies and antigen-binding fragments thereof comprising N-linked glycans that are typically added to immunoglobulins produced in Chinese hamster ovary cells (CHO N-linked glycans) or in engineered yeast cells (engineered yeast N-linked glycans), such as, for example, Pichia pastoris. For example, in an embodiment, the anti-ILT4 antibodies and antigen-binding fragments thereof comprise one or more of the “engineered yeast N-linked glycans” or “CHO N-linked glycans” (e.g., G0 and/or G0-F and/or G1 and/or G1-F and/or G2-F and/or Man5). In an embodiment, the anti-ILT4 antibodies and antigen-binding fragments thereof comprise the engineered yeast N-linked glycans, i.e., G0 and/or G1 and/or G2, optionally, further including Man5. In an embodiment, the anti-ILT4 antibodies and antigen-binding fragments thereof comprise the CHO N-linked glycans, i.e., G0-F, G1-F and G2-F, optionally, further including G0 and/or G1 and/or G2 and/or Man5. In an embodiment, about 80% to about 95% (e.g., about 80-90%, about 85%, about 90% or about 95%) of all N-linked glycans on the anti-ILT4 antibodies and antigen-binding fragments thereof are engineered yeast N-linked glycans or CHO N-linked glycans. See Nett et al. Yeast. 28(3): 237-252 (2011); Hamilton et al. Science. 313(5792): 1441-1443 (2006); Hamilton et al. Curr Opin Biotechnol. 18(5): 387-392 (2007). For example, in an embodiment, an engineered yeast cell is GFI5.0 or YGLY8316 or strains set forth in U.S. Pat. No. 7,795,002 or Zha et al. Methods Mol Biol. 988:31-43 (2013). See also international patent application publication no. WO2013/066765.

The anti-ILT4 antibodies and antigen-binding fragments thereof that can be used with various formulations disclosed herein (e.g., 1E1, 2A6, 3G7 and/or 2C1) can also be engineered to include modifications within the Fc region, typically to alter one or more functional properties of the antibody, such as serum half-life, complement fixation, Fc receptor binding, and/or effector function (e.g., antigen-dependent cellular cytotoxicity). Furthermore, the antibodies and antigen-binding fragments thereof that can be used with various formulations disclosed herein (e.g., IE1, 2A6, 3G7 and/or 2C1) can be chemically modified (e.g., one or more chemical moieties can be attached to the antibody, such as a labeled antibody or an antibody-drug conjugate) or be modified to alter its glycosylation, again to alter one or more fumctional properties of the antibody.

### PD-1 Antibodies and Antigen Binding Fragments Thereof

In one aspect, the invention provides stable biological formulations comprising anti-ILT4 antibodies or antigen binding fragments thereof, co-formulated with an anti-human PD-1 antibodies or antigen binding fragments thereof which specifically bind to human PD-1 (e.g., a human or humanized anti-PD-1 antibody) as the active pharmaceutical ingredient (PD-1 API), as well as methods for using the formulations of the invention. Any anti-PD-1 antibody or antigen binding fragment thereof can be used in the co-formulations and methods of the invention. In particular embodiments, the PD-1 API is an anti-PD-1 antibody, which is selected from pembrolizumab and nivolumab. In specific embodiments, the anti-PD-1 antibody is pembrolizumab. In alternative embodiments, the anti-PD-1 antibody is nivolumab. Table 3 provides amino acid sequences for exemplary anti-human PD-1 antibodies pembrolizumab and nivolumab. Alternative PD-1 antibodies and antigen-binding fragments that are useful in the co-formulations and methods of the invention are shown in Table 4.

In some embodiments, an anti-human PD-1 antibody or antigen binding fragment thereof for use in the co-formulations of the invention comprises three light chain CDRs of CDRL1, CDRL2 and CDRL3 and/or three heavy chain CDRs of CDRH1, CDRH2 and CDRH3.

In one embodiment of the invention, CDRL1 is SEQ ID NO:95 or a variant of SEQ ID NO:95, CDRL2 is SEQ ID NO:96 or a variant of SEQ ID NO:96, and CDRL3 is SEQ ID NO:97 or a variant of SEQ ID NO:97.

In one embodiment, CDRH1 is SEQ ID NO:100 or a variant of SEQ ID NO:100, CDRH2 is SEQ ID NO: 101 or a variant of SEQ ID NO:101, and CDRH3 is SEQ ID NO:102 or a variant of SEQ ID NO:102.

In one embodiment, the three light chain CDRs are SEQ ID NO:95, SEQ ID NO:96, and SEQ ID NO:97 and the three heavy chain CDRs are SEQ ID NO:100, SEQ ID NO:101 and SEQ ID NO:102.

In an alternative embodiment of the invention, CDRL1 is SEQ ID NO:105 or a variant of SEQ ID NO:105, CDRL2 is SEQ ID NO:106 or a variant of SEQ ID NO:106, and CDRL3 is SEQ ID NO:107 or a variant of SEQ ID NO:107.

In one embodiment, CDRH1 is SEQ ID NO:110 or a variant of SEQ ID NO:110, CDRH2 is SEQ ID NO:111 or a variant of SEQ ID NO:111, and CDRH3 is SEQ ID NO:11128 or a variant of SEQ ID NO:112.

In one embodiment, the three light chain CDRs are SEQ ID NO:95, SEQ ID NO:96, and SEQ ID NO:97 and the three heavy chain CDRs are SEQ ID NO:100, SEQ ID NO:101 and SEQ ID NO:102.

In an alternative embodiment, the three light chain CDRs are SEQ ID NO:105, SEQ ID NO:106, and SEQ ID NO:107 and the three heavy chain CDRs are SEQ ID NO:110, SEQ ID NO:111 and SEQ ID NO:112.

In a further embodiment of the invention, CDRL1 is SEQ ID NO:115 or a variant of SEQ ID NO:115, CDRL2 is SEQ ID NO:116 or a variant of SEQ ID NO:116, and CDRL3 is SEQ ID NO:117 or a variant of SEQ ID NO:117.

In yet another embodiment. CDRH1 is SEQ ID NO:118 or a variant of SEQ ID NO:118, CDRH2 is SEQ ID NO: 119 or a variant of SEQ ID NO:119, and CDRH3 is SEQ ID NO:120 or a variant of SEQ ID NO:120.

In another embodiment, the three light chain CDRs are SEQ ID NO:115, SEQ ID NO:116, and SEQ ID NO: 117 and the three heavy chain CDRs are SEQ ID NO:118, SEQ ID NO:119 and SEQ ID NO:120.

Some anti-human PD-1 antibody and antigen binding fragments of the invention comprise a light chain variable region and a heavy chain variable region. In some embodiments, the light chain variable region comprises SEQ ID NO:98 or a variant of SEQ ID NO:98, and the heavy chain variable region comprises SEQ ID NO:103 or a variant of SEQ ID NO:103. In further embodiments, the light chain variable region comprises SEQ ID NO:108 or a variant of SEQ ID NO:108, and the heavy chain variable region comprises SEQ ID NO:113 or a variant of SEQ ID NO:113. In further embodiments, the heavy chain variable region comprises SEQ ID NO:121 or a variant of SEQ ID NO:121 and the light chain variable region comprises SEQ ID NO:122 or a variant of SEQ ID NO:122, SEQ ID NO:123 or a variant of SEQ ID NO:123, or SEQ ID NO:124 or a variant of SEQ ID NO:124. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the co-formulations of the invention, the anti-human PD-1 antibody or antigen binding fragment comprises a light chain variable region comprising or consisting of SEQ ID NO:98 and a heavy chain variable region comprising or consisting of SEQ ID NO:103. In a further embodiment, the anti-human PD-1 antibody or antigen binding fragment comprises a light chain variable region comprising or consisting of SEQ ID NO:108 and a heavy chain variable region comprising or consisting of SEQ ID NO:113. In one embodiment of the formulations of the invention, the anti-human PD-1 antibody or antigen binding fragment comprises a light chain variable region comprising or consisting of SEQ ID NO:122 and a heavy chain variable region comprising or consisting of SEQ ID NO:121. In a further embodiment, the anti-human PD-1 antibody or antigen binding fragment comprises a light chain variable region comprising or consisting of SEQ ID NO:123 and a heavy chain variable region comprising or consisting of SEQ ID NO:121. In another embodiment, the antibody or antigen binding fragment comprises a light chain variable region comprising or consisting of SEQ ID NO:124 and a heavy chain variable region comprising or consisting of SEQ ID NO:121.

In another embodiment, the co-formulations of the invention comprise an anti-human PD-1 antibody or antigen binding protein that has a VL domain and/or a VH domain with at least 99%, 98%, 97%, 96%, 95%, 90%, 85%, 80%, 75% or 50% sequence homology to one of the VL domains or VH domains described above, and exhibits specific binding to PD-1. In another embodiment, the anti-human PD-1 antibody or antigen binding protein of the co-formulations of the invention comprises VL and VH domains having up to 1, 2, 3, 4, or 5 or more amino acid substitutions, and exhibits specific binding to PD-1.

In any of the embodiments above, the PD-1 API may be a full-length anti-PD-1 antibody or an antigen binding fragment thereof that specifically binds human PD-1. In certain embodiments, the PD-1 API is a full-length anti-PD-1 antibody selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the VL and VH regions provided herein. For example, if a particular intended use of an antibody (or fragment) of the invention was to call for altered effector functions, a heavy chain constant domain other than IgG1 may be used. Although IgG1 antibodies provide a long half-life and for effector functions, such as complement activation and antibody-dependent cellular cytotoxicity, such activities may not be desirable for all uses of the antibody. In such instances an IgG4 constant domain, for example, may be used.

In embodiments of the invention, the PD-1 API is an anti-PD-1 antibody comprising a light chain comprising or consisting of a sequence of amino acid residues as set forth in SEQ ID NO:99 and a heavy chain comprising or consisting of a sequence of amino acid residues as set forth in SEQ ID NO:104. In alternative embodiments, the PD-1 API is an anti-PD-1 antibody comprising a light chain comprising or consisting of a sequence of amino acid residues as set forth in SEQ ID NO:109 and a heavy chain comprising or consisting of a sequence of amino acid residues as set forth in SEQ ID NO:114. In further embodiments, the PD-1 API is an anti-PD-1 antibody comprising a light chain comprising or consisting of a sequence of amino acid residues as set forth in SEQ ID NO:126 and a heavy chain comprising or consisting of a sequence of amino acid residues as set forth in SEQ ID NO:31251. In additional embodiments, the PD-1 API is an anti-PD-1 antibody comprising a light chain comprising or consisting of a sequence of amino acid residues as set forth in SEQ ID NO:127 and a heavy chain comprising or consisting of a sequence of amino acid residues as set forth in SEQ ID NO:125. In yet additional embodiments, the PD-1 API is an anti-PD-1 antibody comprising a light chain comprising or consisting of a sequence of amino acid residues as set forth in SEQ ID NO:128 and a heavy chain comprising or consisting of a sequence of amino acid residues as set forth in SEQ ID NO:125. In some co-formulations of the invention, the PD-1 API is pembrolizumab or a pembrolizumab biosimilar. In some co-formulations of the invention, the PD-1 API is nivolumab or a nivolumab biosimilar.

Ordinarily, amino acid sequence variants of the anti-PD-1 antibodies and antigen binding fragments of the invention and the anti-ILT4 antibodies and antigen binding fragments will have an amino acid sequence having at least 75% amino acid sequence identity with the amino acid sequence of a reference antibody or antigen binding fragment (e.g., heavy chain, light chain, VH, VL, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70, Henikoff, S., et al., (1992) Proc. Natl. Acad. Sci. USA 89:10915-10919; Altschul, S. F., et al., (1993) J1 Mol. Evol. 36:290-300; ALIGNMENT STATISTICS: Karlin, S., t al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

The formulation further comprises an anti-PD-1 antibody or antigen binding fragment as exemplified below.

TABLE 3
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 95 |
| CDR2 | LASYLES | 96 |
| CDR3 | QHSRDLPLT | 97 |
| Variable | EIVLTQSPATLSLSPGERATLSCRASKGVSTSGYSYLHWY | 98 |
| Region | QQKPGQAPRLLIYLASYLESGVPARFSGSGSGTDFTLTISS | |
| | LEPEDFAVYYCQHSRDLPLTFGGGTKVEIK | |
| Light Chain | EIVLTQSPATLSLSPGERATLSCRASKGVSTSGYSYLHWY | 99 |
| | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQ | |
| | SGNSQESVTEQDSKDSTYSLSSTLTLSKADYEKHKVYAC | |
| | EVTHQGLSSPVTKSFNRGEC | |

Pembrolizumab Heavy Chain

| | | |
|---|---|---|
| CDR1 | NYYMY | 100 |
| CDR2 | GINPSNGGTNFNEKFKN | 101 |
| CDR3 | RDYRFDMGFDY | 102 |
| Variable | QVQLVQSGVEVKKPGASVKVSCKASGYTFTNYYMYWV | 103 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 104 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |

Nivolumab Light Chain

| | | |
|---|---|---|
| CDR1 | RASQSVSSYLA | 105 |
| CDR2 | DASNRAT | 106 |
| CDR3 | QQSSNWPRT | 107 |
| Variable | EIVLTQSPATLSLSPGERATLSCRASQSVSSYLAWYQQKP | 108 |
| Region | GQAPRLLIYDASNRATGIPARFSGSGSGTDFTLTISSLEPE | |
| | DFAVYYCQQSSNWPRTFGQGTKVEIK | |
| Light Chain | EIVLTQSPATLSLSPGERATLSCRASQSVSSYLAWYQQKP | 109 |
| | GQAPRLLIYDASNRATGIPARFSGSGSGTDFTLTISSLEPE | |
| | DFAVYYCQQSSNWPRTFGQGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |

Nivolumab Heavy Chain

| | | |
|---|---|---|
| CDR1 | NSGMH | 110 |
| CDR2 | VIWYDGSKRYYADSVKG | 111 |
| CDR3 | NDDY | 112 |
| Variable | QVQLVESGGGVVQPGRSLRLDCKASGITFSNSGMHWVR | 113 |
| Region | QAPGKGLEWVAVIWYDGSKRYYADSVKGRFTISRDNSK | |
| | NTLFLQMNSLRAEDTAVYYCATNDDYWGQGTLVTVSS | |
| Heavy | QVQLVESGGGVVQPGRSLRLDCKASGITFSNSGMHWVR | 114 |
| Chain | QAPGKGLEWVAVIWYDGSKRYYADSVKGRFTISRDNSK | |
| | NTLFLQMNSLRAEDTAVYYCATNDDYWGQGTLVTVSSA | |
| | STKGPSVFPLAPCSRSTSESTAALGCLVKDYFPEPVTVSW | |
| | NSGALTSGVHTFPAVLQSSGLYSLSSVVTVPSSSLGTKTY | |
| | TCNVDHKPSNTKVDKRVESKYGPPCPPCPAPEFLGGPSVF | |
| | LFPPKPKDTLMISRTPEVTCVVVDVSQEDPEVQFNWYVD | |
| | GVEVHNAKTKPREEQFNSTYRVVSVLTVLHQDWLNGKE | |
| | YKCKVSNKGLPSSIEKTISKAKGQPREPQVYTLPPSQEEM | |
| | TKNQVSLTCLVKGFYPSDIAVEWESNGQPENNYKTTPPV | |
| | LDSDGSFFLYSRLTVDKSRWQEGNVFSCSVMHEALHNH | |
| | YTQKSLSLSLGK | |

TABLE 4
Additional Anti-PD-1 Antibodies and Antigen Binding Fragments
Useful in the Formulations, Methods and Uses of the Invention.
A. Antibodies and antigen binding fragments comprising light and
heavy chain CDRs of hPD-1.08A in WO2008/156712 (incorporated
herein by reference in its entirety)

| | |
|---|---|
| CDRL1 | SEQ ID NO: 115 |
| CDRL2 | SEQ ID NO: 116 |
| CDRL3 | SEQ ID NO: 117 |
| CDRH1 | SEQ ID NO: 118 |
| CDRH2 | SEQ ID NO: 119 |
| CDRH3 | SEQ ID NO: 120 |

B. Antibodies and antigen binding fragments comprising the mature
h109A heavy chain variable region and one of the mature K09A light
chain variable regions in WO 2008/156712

| | |
|---|---|
| Heavy chain VR | SEQ ID NO: 121 |
| Light chain VR | SEQ ID NO: 122 or |
| | SEQ ID NO: 123 or |
| | SEQ ID NO: 124 |

C. Antibodies and antigen binding fragments comprising the mature
409 heavy chain and one of the mature K09A light chains in WO
2008/156712

| | |
|---|---|
| Heavy chain | SEQ ID NO: 125 |
| Light chain | SEQ ID NO: 126 or |
| | SEQ ID NO: 127 or |
| | SEQ ID NO: 128 |

As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

In another aspect of the invention, the formulation comprises an anti-ILT4 antibody or antigen binding fragment comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21); and an anti-PD-1 antibody or antigen binding fragment comprising a light chain variable region sequence of SEQ ID NO: 98 and a heavy chain variable region sequence of SEQ ID NO: 103. In another embodiment, the formulation comprises an anti-ILT4 antibody comprising wherein the anti-ILT4 antibody or antigen binding fragment thereof comprises a heavy chain variable domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GDSNRPS (SEQ ID NO: 52), and CDR-L3: QSFDNSLSAYV (SEQ ID NO: 21) and an anti-PD-1 antibody comprising a light chain sequence of SEQ ID NO: 5 and a heavy chain sequence of SEQ ID NO: 104. The invention also provides formulations of anti-ILT4 antibodies or antigen binding fragments thereof a heavy chain variable domain comprising the amino acid sequence set forth in SEQ ID NO:57 and a light chain variable domain comprising the amino acid sequence set forth in SEQ ID NO:58; and an anti-PD-1 antibody comprising a light chain CDRL1 sequence of SEQ ID NO: 95, CDRL2 sequence of SEQ ID NO: 96, and CDRL3 sequence of SEQ ID NO: 97, and a heavy chain CDRH1 sequence of SEQ ID NO: 100, CDRH2 sequence of SEQ ID NO: 101, and CDRH3 sequence of SEQ ID NO: 102. In some formulations of the invention, the anti-ILT4 antibody comprises a heavy chain comprising the amino acid sequence set forth in SEQ ID NO:2 and a light chain comprising the amino acid sequence set forth in SEQ ID NO:7. In some embodiments, the anti-ILT4 antibody or antigen binding fragment thereof comprises a heavy chain comprising the amino acid sequence set forth in SEQ ID NO:80 and a light chain comprising the amino acid sequence set forth in SEQ ID NO:7.

In a further aspect of the invention, the formulations comprise an anti-ILT4 antibody or antigen binding fragment thereof comprises a heavy chain variable domain consisting of the amino acid sequence set forth in SEQ ID NO:57 and a light chain variable domain consisting of the amino acid sequence set forth in SEQ ID NO:58, and an anti-PD-1 antibody or antigen binding fragment that comprises a light chain variable region sequence of SEQ ID NO: 108 and a heavy chain variable region sequence of SEQ ID NO: 113. The invention also provides formulations of an anti-ILT4 antibody or antigen binding fragment comprising comprises a heavy chain consisting of the amino acid sequence set forth in SEQ ID NO:80 and a light chain consisting of the amino acid sequence set forth in SEQ ID NO:7, and an anti-PD-1 antibody or antigen binding fragment comprising a light chain variable region CDRL1 sequence of SEQ ID NO: 105, CDRL2 sequence of SEQ ID NO: 106, and CDRL3 sequence of SEQ ID NO: 107, and a heavy chain variable region CDRH1 sequence of SEQ ID NO: 110, CDRH2 sequence of SEQ ID NO: 111, and CDRH3 sequence of SEQ ID NO: 112.

Antibody or antigen binding fragments of the formulation can comprise a light chain variable region and a heavy chain variable region. In some embodiments, the light chain variable region comprises SEQ ID NO:98 or a variant of SEQ ID NO:98, and the heavy chain variable region comprises SEQ ID NO:103 or a variant of SEQ ID NO:103. In further embodiments, the light chain variable region comprises SEQ ID NO:108 or a variant of SEQ ID NO:108, and the heavy chain variable region comprises SEQ ID NO:113 or a variant of SEQ ID NO:113. In further embodiments, the heavy chain variable region comprises SEQ ID NO:121 or a variant of SEQ ID NO:121 and the light chain variable region comprises SEQ ID NO:122 or a variant of SEQ ID NO:1228, SEQ ID NO:123 or a variant of SEQ ID NO:123, or SEQ ID NO:124 or a variant of SEQ ID NO:124. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In another embodiment, the formulations of the invention comprise an antibody or antigen binding fragment that has a V_{L} domain and/or a V_{H} domain with at least 95%, 90%, 85%, 80%, 75% or 50% sequence homology to one of the V_{L} domains or V_{H} domains described above, and exhibits specific binding to PD-1 or ILT4. In another embodiment, the antibody or antigen binding fragment of the formulations of the invention comprises V_{L} and V_{H} domains having up to 1, 2, 3, 4, or 5 or more amino acid substitutions, and exhibits specific binding to PD-1 or ILT4.

In embodiments of the invention, the antibody is an anti-PD-1 antibody comprising a light chain comprising or consisting of a sequence of amino acid residues as set forth in SEQ ID NO:99 and a heavy chain comprising or consisting of a sequence of amino acid residues as set forth in SEQ ID NO:104. In alternative embodiments, the antibody is an anti-PD-1 antibody comprising a light chain comprising or consisting of a sequence of amino acid residues as set forth in SEQ ID NO:109 and a heavy chain comprising or consisting of a sequence of amino acid residues as set forth in SEQ ID NO:114. In further embodiments, the antibody is an anti-PD-1 antibody comprising a light chain comprising or consisting of a sequence of amino acid residues as set forth in SEQ ID NO:126 and a heavy chain comprising or consisting of a sequence of amino acid residues as set forth in SEQ ID NO:125. In additional embodiments, the antibody is an anti-PD-1 antibody comprising a light chain comprising or consisting of a sequence of amino acid residues as set forth in SEQ ID NO:127 and a heavy chain comprising or consisting of a sequence of amino acid residues as set forth in SEQ ID NO:125. In yet additional embodiments, the antibody is an anti-PD-1 antibody comprising a light chain comprising or consisting of a sequence of amino acid residues as set forth in SEQ ID NO:128 and a heavy chain comprising or consisting of a sequence of amino acid residues as set forth in SEQ ID NO:125. In some formulations of the invention, the anti-PD-1 antibody is pembrolizumab or a pembrolizumab variant.

Ordinarily, amino acid sequence variants of the anti-PD-1 or anti-ILT4 antibodies and antigen binding fragments of the invention will have an amino acid sequence having at least 75% amino acid sequence identity with the amino acid sequence of a reference antibody or antigen binding fragment (e.g., heavy chain, light chain, V_{H}, V_{L}, framework or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 or anti-ILT4 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

In one embodiment, the ratio of anti-ILT4 antibody to anti-PD-1 antibody in the formulation is 1:1, 1:2 or 1:3. In another embodiment, the molar ratio of anti-ILT4 antibody to anti-PD-1 antibody in the formulation is 1:1, 2:1, 3:1, 3.5:1, 4:1, 5:1 or 6:1. In one embodiment, the molar ratio of anti-ILT4 antibody to anti-PD-1 antibody in the formulation is 4:1. In another embodiment, the molar ratio of anti-ILT4 antibody to anti-PD-1 antibody in the formulation is 5:1.

### Formulations

The coformulations described herein can retain physical, chemical, and/or biological stability of an anti-ILT4 antibody (e.g., 1E1, 2A6, 3G7, or 2C1) or antigen-binding fragment thereof during storage (e.g., at about 5° C. for at least 6 months, 12 months, 24 months, or 36 months) and/or under various stress conditions (e.g., agitation, freeze and thaw cycles, or light exposure).

In one aspect, provided herein are various co-formulations of an anti-ILT4 antibody (e.g., 1E1, 2A6, 307, or 2C1) or antigen-binding fragment thereof and anti-PD-1 antibody or antigen-binding fragment thereof, comprising (i) an anti-ILT4 antibody or antigen-binding fragment thereof (e.g., 1E1, 2A6, 3G7, or 2C1); (ii) an anti-PD-1 antibody or antigen binding fragment thereof; (iii) a buffer (e.g., L-histidine buffer or acetate buffer); (iv) a non-reducing sugar (e.g., sucrose); (v) a non-ionic surfactant (e.g., PS-80); and (vi) an antioxidant (e.g., L-methionine).

In some aspects of the invention, the formulations of the invention minimize the formation of antibody aggregates (high molecular weight species) and particulates, improve colloidal stability, minimize fragmentation (low molecular weight species), or insure that the antibody maintains its biological activity over time. In one aspect, the formulation comprises: about 3-300 mg/mL of an anti-ILT4 antibody or antigen-binding fragment thereof and about 3-300 mg/mL of an anti-PD-1 antibody or antigen-binding fragment thereof at a molar ratio of 4:1 to 5:1 (anti-ILT4 antibody to anti-PD-1 antibody, or antigen binding fragments thereof), one or more of an excipient selected from the group consisting of histidine, aspartate, glutamine, glycine, proline, methionine, arginine or pharmaceutically acceptable salt thereof, NaCl, KCl, LiCl, CaCl₂, MgCl₂, ZnCl₂, and FeCl₂, at a total excipient concentration of about 10-1000 mM, and a buffer at pH about 5-8. In one embodiment, the anti-ILT4 antibody or antigen-binding fragment thereof and anti-PD-1 antibody or antigen-binding fragment thereof have a molar ratio of 4:1 (anti-ILT4 antibody to anti-PD-1 antibody, or antigen binding fragments thereof). In another embodiment, the anti-ILT4 antibody or antigen-binding fragment thereof and anti-PD-1 antibody or antigen-binding fragment thereof have a molar ratio of 5:1 (anti-ILT4 antibody to anti-PD-1 antibody, or antigen binding fragments thereof). In another aspect, the formulation comprises: about 4-200 mg/mL of an anti-ILT4 antibody or antigen-binding fragment thereof and about 4-200 mg/ml of an anti-PD-1 antibody or antigen-binding fragment thereof. In one embodiment, one or more of an excipient selected from the group consisting of histidine, aspartate, glutamine, glycine, proline, methionine, arginine or a pharmaceutically acceptable salt thereof, NaCl, KCl, LiCl, CaCl₂, MgCl₂, ZnCl₂, and FeCl₂, is at a total excipient concentration of about 25-250 mM. In another embodiment, one or more of an excipient selected from the group consisting of histidine, aspartate, glutamine, glycine, proline, methionine, arginine or pharmaceutically acceptable salt thereof, NaCl, KCl, LiCl, CaCl₂, MgCl₂, ZnCl₂, and FeCl₂, is at a total excipient concentration of about 40-250 mM.

In one aspect, the excipient is arginine or a pharmaceutically acceptable salt thereof at a concentration of about 15-250 mM. In one aspect, the excipient is arginine or a pharmaceutically acceptable salt thereof at a concentration of about 25-250 mM. In another embodiment, the excipient is arginine or a pharmaceutically acceptable salt thereof at a concentration of about 40-150 mM. In another embodiment, the excipient is arginine or a pharmaceutically acceptable salt thereof at a concentration of about 40-100 mM. In another embodiment, the excipient is L-arginine or a pharmaceutically acceptable salt thereof at a concentration of about 70 mM. In another embodiment, the excipient is arginine or a pharmaceutically acceptable salt thereof at a concentration of about 70-150 mM. Examples of pharmaceutically acceptable salts of arginine (L or D form) include but are not limited to L-arginine-hydrochloride and L-arginine succinate. In other aspects of the foregoing embodiments, the formulation further comprises a non-ionic surfactant, sugar or polyol, or glutamine, glycine, proline, or methionine.

In another aspect, the excipients are NaCl and arginine or a pharmaceutically acceptable salt thereof with a total excipient concentration of about 25-250 mM. In a further embodiment, the excipients are NaCl and arginine or a pharmaceutically acceptable salt thereof with a total excipient concentration of about 70-100 mM. In one embodiment, the NaCl to arginine concentration ratio is 1:1. In another embodiment, the NaCl concentration is about 35 mM and the arginine concentration is about 35 mM. In another embodiment, the NaCl concentration is about 50 mM and the arginine concentration is about 50 mM.

In a further aspect, the excipient is NaCl, KCl or LiCl at about 40-150 mM. In a further embodiment, the excipient is NaCl, KCl or LiCl at about 40-100 mM. In a further embodiment, the excipient is NaCl, KCl or LiCl at about 70-130 mM. In a further embodiment, the excipient is NaCl, KCl or LiCl at about 70-100 mM. In a further embodiment, the excipient is NaCl at about 70 mM. In other aspects of the foregoing embodiments, the formulation further comprises a non-ionic surfactant.

In a further aspect, the excipient is L-histidine at about 25-200 mM. In a further embodiment, the L-histidine is at about 50-200 mM. In yet a further embodiment, the L-histidine is at about 40-100 mM.

In a further aspect, the excipient is L-glutamine, L-glycine, L-proline or L-methionine, or a combination thereof at about 25-200 mM. In a further embodiment, the excipient is at about 50-200 mM. In yet a further embodiment, the excipient is at about 40-100 mM. In yet a further embodiment, the excipient is at about 70 mM.

In one embodiment, the excipient is L-glutamine, L-glycine, L-aspartate, or a combination thereof at about 25-200 mM. In another embodiment, the excipient is at about 20-50 mM. In a further embodiment, the excipient is at about 20 mM. In yet a further embodiment, the excipient is at about 40-100 mM. In yet a further embodiment, the excipient is at about 70 mM. In another embodiment, the excipients are about 20 mM L-aspartate and about 50 mM L-glycine. In another embodiment, the excipients are about 20 mM L-glutamine and about 50 mM L-glycine.

In one embodiment, the co-formulated composition has a buffer having a neutral or slightly acidic pH (pH 4.5-8), and arginine or a pharmaceutically acceptable salt thereof. In one embodiment, a buffer of pH about 5.5-6.5 is used in the composition. In one embodiment, a buffer of pH about 4.5-6.5 is used in the composition. In another embodiment, a buffer of pH about 5.5-6.0 is used in the composition. In a further embodiment, a buffer of pH about 5.0-6.0 is used in the composition. The buffer can have a concentration of about 5-1000 mM. In another embodiment, the buffer can have a concentration of about 5-150 mM. In a further embodiment, the buffer can have a concentration of about 5-300 mM. In a further embodiment, the buffer has a concentration of about 1-300 mM. In another embodiment, the buffer can have a concentration of about 1-30 mM. In yet a further embodiment, the buffer can have a concentration of 5-30 mM. In yet a further embodiment, the buffer can have a concentration of about 5-20 mM. In yet a further embodiment, the buffer can have a concentration of about 8-12 mM. In one embodiment, the buffer is histidine, acetate or citrate. In some embodiments, the buffer contains about 10 mM histidine, acetate or citrate.

In one embodiment, the formulation comprises about 3-300 mg/mL of an anti-ILT4 antibody or antigen-binding fragment thereof and 3-300 mg/mL of an anti-PD-1 antibody or antigen-binding fragment thereof at a molar ratio of 4:1 to 5:1 (anti-ILT4 antibody to anti-PD-1 antibody, or antigen binding fragments thereof), sugar or polyol; a non-ionic surfactant, a histidine buffer or acetate buffer at pH about 4.5-8, about 10-1000 mM methionine (L or D form) or a pharmaceutically acceptable salt thereof and optionally arginine, EDTA, DTPA, tryptophan (L or D form) or pyridoxine. In another embodiment, the formulation comprises about 4-250 mg/mL of an anti-ILT4 antibody or antigen-binding fragment thereof and about 4-250 mg/mL of an anti-PD-1 antibody or antigen-binding fragment thereof at a molar ratio of 4:1 to 5:1 (anti-ILT4 antibody to anti-PD-1 antibody, or antigen binding fragments thereof), a sugar or polyol; a non-ionic surfactant, about 50-500 mM histidine buffer at pH about 5-8, about 10-1000 mM salt of monovalent cations selected from NaCl, KCl and LiCl or salt of polyvalent cations selected from CaCl₂, MgCl₂, ZnCl₂, FeCl₂ and FeCl₃, optionally about 10-1000 mM methionine (D or L form) or a pharmaceutically acceptable salt thereof and optionally arginine, EDTA, DTPA, tryptophan and Pyridoxine. In another aspect, the formulation comprises: about 4-200 mg/mL of an anti-ITL4 antibody or antigen-binding fragment thereof and about 4-200 mg/ml of an anti-PD-1 antibody or antigen-binding fragment thereof. In one embodiment, the anti-ILT4 antibody or antigen-binding fragment thereof and anti-PD-1 antibody or antigen-binding fragment thereof has a molar ratio of 4:1 (anti-ILT4 antibody to anti-PD-1 antibody, or antigen binding fragments thereof). In another embodiment, the anti-ILT4 antibody or antigen-binding fragment thereof and anti-PD-1 antibody or antigen-binding fragment thereof has a molar ratio of 5:1 (anti-ILT4 antibody to anti-PD-1 antibody, or antigen binding fragments thereof).

The formulation may include about 1-100 uM, about 1-30 uM, about 1-20 uM, about 10 uM-30 uM DTPA or EDTA. The formulation may also include about 1-30 mM L-methionine. In one embodiment, the formulation may also include about 1-20 mM L-methionine. The formulation may also include about 5-15 mM L-methionine. The formulation may also include about 5-15 mM L-methionine. The formulation may also include about 5-20 mM L-methionine. The formulation may also include about 10 mM, or at least about 10 mM L-methionine. Sometimes nitrogen overlay (blanketing, for example only 5% or 10% residual O₂ upon nitrogen overlay) is used during production steps and/or prior to vial closure, to stabilize antibody against oxidation.

In another aspect of the invention, the formulation further comprises a sugar, polyol, or a non-ionic surfactant, or a combination thereof. In one embodiment, the sugar is selected from the group consisting of glucose, sucrose, trehalose and lactose or a combination thereof. In one embodiment, the sugar is a disaccharide such as sucrose, trehalose and maltose. In one embodiment, the sugar is a non-reducing sugar. In another embodiment, the sugar is a non-reducing disaccharide such as sucrose or trehalose, or a combination thereof. In one embodiment, the sugar is at a concentration of about 10-200 mg/ml. In another embodiment, the sugar is at a concentration of about 30-120 mg/ml. In another embodiment, the sugar is at a concentration of about 30-80 mg/ml. In a further embodiment, the sugar is at a concentration of about 50-90 mg/ml.

In one embodiment, the polyol is selected from the group consisting of mannitol, sorbitol, glycerol and polyethylene glycol. In another embodiment, the polyol is a sugar alcohol. In one embodiment, the sugar and polyol are selected from the group consisting of sucrose, trehalose, sorbitol, glycerol and polyethylene glycol. In a further embodiment, the polyol is a glycol. In one embodiment, the glycol is selected from the group consisting of ethylene glycol, propylene glycol and polyethylene glycol. In one embodiment, the polyol is at a concentration of about 10-200 mg/ml. In another embodiment, the polyol is at a concentration of about 10-50 mg/ml. In a further embodiment, the polyol is at a concentration of about 5-30 mg/ml.

In one embodiment, the formulation comprises about 10-250 mg/ml of sucrose or trehalose. In another embodiment, the formulation comprises about 20-200 mg/ml of sucrose or trehalose. In a further embodiment, the formulation comprises about 50-80 mg/ml of sucrose or trehalose. In a further embodiment, the formulation comprises about 30-80 mg/ml of sucrose or trehalose. In another embodiment, the formulation comprises about 50-90 mg/ml of sucrose or trehalose. In yet a further embodiment, the formulation comprises about 70-80 mg/ml of sucrose or trehalose. In yet a further embodiment, the formulation comprises at least about 50 mg/ml of sucrose or trehalose. In another embodiment, the formulation comprises about 20-200 mg/ml of sorbitol, PEG400 or glycerol. In a further embodiment, the formulation comprises about 20-50 mg/ml of sorbitol, PEG400 or glycerol.

In one embodiment, the non-ionic surfactant is selected from the group consisting of a polysorbate and a poloxamer. In yet another embodiment, the surfactant is selected from the group consisting of Tween80® (polysorbate 80), Tween20® (polysorbate 20), PluronicF88®, Pluoronic F-127®, PluronicF68®, Triton X-100®. In one embodiment, the surfactant is polysorbate 20 or polysorbate 80, and the sugar is sucrose or trehalose. The polysorbate 80 or polysorbate 20 surfactant may be present in the formulation in an amount from about 0.005 to about 1 mg/ml. The polysorbate 80 or polysorbate 20 surfactant may be present in the formulation in an amount from about 0.02 to about 2 mg/ml. The polysorbate 80 or polysorbate 20 surfactant may be present in the formulation in an amount from about 0.05 to about 1 mg/ml. The polysorbate 80 or polysorbate 20 surfactant may be present in the formulation in an amount from about 0.1 to about 0.5 mg/ml. In another embodiment, the polysorbate 80 or polysorbate 20 surfactant may be present in the formulation in an amount from about at least about 0.005 mg/ml. The polysorbate 80 or polysorbate 20 surfactant may also be present in the formulation in an amount from about at least about 0.1 mg/ml. The polysorbate 80 surfactant may be present in the formulation in an amount from about 0.2 mg/ml.

In other aspects of the above formulations, at 50° C., the % High Molecular Weight (HMW) is less than 5% in the co-formulated anti-ILT4 antibody and anti-PD-1 antibody formulation after 10-days as measured by size exclusion chromatography.

### Liquid Formulation

A liquid antibody formulation can be made by taking the drug substance, which is in, for example, in an aqueous pharmaceutical formulation and buffer exchanging it into the desired buffer as the last step of the purification process. The drug substance in the final buffer is concentrated to a desired concentration. Excipients such as stabilizers and surfactants are added to the drug substance and it is diluted using the appropriate buffer to final protein concentration. The final formulated drug substance is filtered using 0.22 μm filters and filled into a final container (e.g., glass vials). The formulation may be stored in a vial, and delivered through an injection device or vessel.

In another aspect of the invention, for the liquid co-formulated formulation comprising the anti-PD-1 antibody and anti-ILT4 antibody (or antigen binding fragments thereof), the anti-ILT4 antibody or antigen binding fragment thereof has the concentration of about 10-300 mg/ml. In another embodiment, the anti-ILT4 antibody or antigen binding fragment thereof has the concentration of about 15-250 mg/ml. In another embodiment, the anti-ILT4 antibody or antigen binding fragment thereof has the concentration of about 20-100 mg/ml. In a further embodiment, the anti-ILT4 antibody or antigen binding fragment thereof has the concentration of about 30-80 mg/ml. In a further embodiment, the anti-ILT4 antibody or antigen binding fragment thereof has the concentration of about 35-75 mg/ml. In a further embodiment, the anti-ILT4 antibody or antigen binding fragment thereof has the concentration of about 40-70 mg/mL. In a further embodiment, the anti-ILT4 antibody or antigen binding fragment thereof has the concentration of about 45-65 mg/ml. In another embodiment, the anti-ILT4 antibody or antigen binding fragment thereof is at a concentration of about 45-60 mg/mL. In yet a further embodiment, the anti-ILT4 antibody or antigen binding fragment thereof is at a concentration of about 45-65 or 55 mg/mL. In yet a further embodiment, the anti-ILT4 antibody or antigen binding fragment thereof is at a concentration of about 40-60 mg/mL or 45-55 mg/mL. In yet a further embodiment, the anti-ILT4 antibody or antigen binding fragment thereof is at a concentration of about 46-52 mg/mL. In yet a further embodiment, the anti-ILT4 antibody or antigen binding fragment thereof is at a concentration of about 48-52 mg/mL. In yet a further embodiment, the anti-ILT4 antibody or antigen binding fragment thereof is at a concentration of about 49 mg/mL. In yet a further embodiment, the anti-ILT4 antibody or antigen binding fragment thereof is at a concentration of about 50 mg/mL. In yet a further embodiment, the anti-ILT4 antibody or antigen binding fragment thereof is at a concentration of about 51 mg/mL.

In another aspect of the invention, the anti-PD-1 antibody or antigen binding fragment thereof in the liquid formulation has the concentration of about 3-300 mg/ml. In one embodiment, the anti-PD-1 antibody or antigen binding fragment thereof is at concentration of about 4-250 mg/ml. In another embodiment, the anti-PD-1 antibody or antigen binding fragment thereof is at a concentration of about 40-100 mg/ml. In a further embodiment, the anti-PD-1 antibody or antigen binding fragment thereof is at a concentration of about 10-60 mg/ml. In a further embodiment, the anti-PD-1 antibody or antigen binding fragment thereof is at a concentration of about 20-30 mg/ml. In a further embodiment, the anti-PD-1 antibody or antigen binding fragment thereof is at a concentration of about 10-30 mg/mL. In a further embodiment, the anti-PD-1 antibody or antigen binding fragment thereof is at a concentration of about 15-50 mg/ml. In another embodiment, the anti-PD-1 antibody or antigen binding fragment thereof is at a concentration of about 10-100 mg/mL. In another embodiment, the anti-PD-1 antibody or antigen binding fragment thereof is at a concentration of about 20-30 or 25 mg/mL. In one embodiment, the anti-PD-1 antibody or antigen binding fragment thereof is at concentration of about 21-29 mg/ml. In one embodiment, the anti-PD-1 antibody or antigen binding fragment thereof is at concentration of about 22-28 mg/ml. In one embodiment, the anti-PD-1 antibody or antigen binding fragment thereof has a concentration of about 24 mg/ml. In one embodiment, the anti-PD-1 antibody or antigen binding fragment thereof has a concentration of about 25 mg/ml. In one embodiment, the anti-PD-1 antibody or antigen binding fragment thereof has a concentration of about 26 mg/ml.

In one embodiment, the liquid formulation comprises a buffer at pH about 4.5-8, 5.0-6.5, 5.5-6.5, 5.5, 5.6, 5.7, 5.8, 5.9, 6.0, 6.1 or 6.2 and arginine or a pharmaceutically acceptable salt thereof. In one embodiment, the liquid formulation comprises a buffer at pH about 5-8. In one embodiment, the liquid formulation comprises a buffer at pH about 5.0-6.5. In one embodiment, the liquid formulation comprises a buffer at pH about 5.0-6.0. In other embodiments, the buffer is histidine. In another embodiment, the buffer is citrate or acetate. In a further embodiment, the liquid formulation comprises an acetate buffer at pH about 5-8, 5.0-6.5, 5.5-6.5, 5.5, 5.6, 5.7, 5.8, 5.9, 6.0, 6.1 or 6.2 and arginine or a pharmaceutically acceptable salt thereof.

The liquid antibody formulation of this invention is suitable for parenteral administration such as intravenous, intramuscular, intraperitoneal, or subcutaneous injection; particularly suitable for subcutaneous injection.

### Dosing and Administration

Toxicity is a consideration in selecting the proper dosing of a therapeutic agent, such as a humanized anti-ILT4 or anti-PD-1 antibody (or antigen binding fragments thereof). Toxicity and therapeutic efficacy of the antibody compositions, administered alone or in combination with an immunosuppressive agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio of LD50 to ED50. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized.

Suitable routes of administration may, for example, include parenteral delivery, including intramuscular, intradermal, subcutaneous, intramedullary injections, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g., subcutaneous administration) require that a lyophilized formulation to enable reconstitution at high concentration.

Alternately, one may administer the antibody in a local rather than systemic manner, for example, via injection of the antibody directly into a pathogen-induced lesion characterized by immunopathology, often in a depot or sustained release formulation. Furthermore, one may administer the antibody in a targeted drug delivery system, for example, in a liposome coated with a tissue-specific antibody, targeting, for example, pathogen-induced lesion characterized by immunopathology. The liposomes will be targeted to and taken up selectively by the afflicted tissue.

Selecting an administration regimen for a therapeutic depends on several factors, including the serum or tissue tumover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002).

Determination of the appropriate dose is made by the clinician, e.g., using parameters or factors known or suspected in the art to affect treatment or predicted to affect treatment. The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. The protein is suitably administered to the patient at one time or repeatedly. The protein may be administered alone or in conjunction with other drugs or therapies.

Antibodies, or antibody fragments can be provided by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects.

In certain embodiments, the pharmaceutical formulations of the invention will be administered by intravenous (IV) infusion or injection.

In other embodiments, the pharmaceutical formulations of the invention will be administered by subcutaneous administration. Subcutaneous administration may be performed by injected using a syringe, or using other injection devices (e.g., the Inject-Ease® device); injector pens; or needleless devices (e.g., MediJector and BioJector®).

Subcutaneous administration may be performed by injection using a syringe, an autoinjector, an injector pen or a needleless injection device. Intravenous injection may be performed after diluting the formulation with suitable commercial diluent such as saline solution or 5% dextrose in water.

Although the high concentration solution formulations of the invention are particularly advantageous for uses requiring a high concentration of antibody, there is no reason that the formulations can't be used at lower concentrations in circumstances where high concentrations are not required or desirable. Lower concentrations of antibody may be useful for low dose subcutaneous administration, or in other modes of administration (such as intravenous administration) where the volume that can be delivered is substantially more than 1 ml. Such lower concentrations can include about 15, 10, 5, 2, 1 mg/ml or less.

### Uses

The invention provides lyophilized or liquid formulations of an anti-ILT4 antibody or antigen-binding fragment thereof and an anti-PD-1 antibody or antigen-binding fragment for use in the treatment of cancer and infection.

Those skilled in the art will realize that the term “cancer” to be the name for diseases in which the body's cells become abnormal and divide without control. Cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma): Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre tumor cervical dysplasia), ovaries (ovarian carcinoma [serous cystadenocarcinoma, mucinous cystadenocarcinoma, unclassified carcinoma], granulosa thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. In one embodiment, the cancer is selected from colorectal cancer, gastric cancer and head and neck cancer.

Buffers that can be used in the pharmaceutical formulations disclosed herein include, but are not limited to, succinate (sodium or potassium), L-histidine, phosphate (sodium or potassium), Tris (tris (hydroxymethyl) aminomethane), diethanolamine, citrate (sodium), acetate (sodium) and the like. In some embodiments of the formulation, the buffer is present in the formulation at a concentration of about 1-20 mM (1, 2, 3, 4, 5, 6, 7, 8, 9, 10, 11, 12, 13, 14, 15, 16, 17, 18, 19, or 20 mM).

The buffer of various formulations described herein has a pH in the range of from about 4.5 to about 7.0, from about 4.5 to about 6.8, from about 5.0 to about 6.8, from about 5.0 to about 6.5, from 5.0 to about 6.0, from about 5.5 to about 6.0. In arriving at the exemplary formulation, L-histidine and acetate buffers in the pH range of 5.0-6.8 were explored for suitability. When a range of pH values is recited, such as “a pH from about pH 5.5 to about 6.0,” the range is intended to be inclusive of the recited values. For example, a range from about 5.0 to about 6.0 includes 5.0, 5.1, 5.2, 5.3, 5.4, 5.5, 5.6, 5.7, 5.8, 5.9, and 6.0. In the event where the anti-ILT4 formulation described herein is a lyophilized formulation, the pH refers to the pH after reconstitution of the lyophilized formulation. pH is typically measured at 25° C. using a standard glass bulb pH meter. As used herein, a solution comprising “histidine buffer at pH X” refers to a solution at pH X and comprising the histidine buffer, i.e., the pH is intended to refer to the pH of the solution.

In some embodiments, the anti-ILT4 formulation comprises a non-reducing sugar. As used herein, “non-reducing sugar” is a sugar not capable of acting as a reducing agent because it does not contain or cannot be converted to contain a free aldehyde group or a free ketone group. Examples of non-reducing sugars include, but are not limited to, disaccharides such as sucrose and trehalose. In an embodiment of the invention, the non-reducing sugar is present in an amount of from about 1% (w/v) to about 10% (w/v) (about 1, 2, 3, 4, 5, 6, 7, 8, 9 or 10% (w/v)). In another embodiment, the non-reducing sugar is present in an amount from about 6% (w/v) to about 8% (w/v) (about 6, 7, or 8% (w/v)). In a further embodiment, the non-reducing sugar is present in an amount of about 6% (w/v). In a further embodiment, the non-reducing sugar is present in an amount of about 7% (w/v). In a further embodiment, the non-reducing sugar is present in an amount of about 8% (w/v). In one embodiment, the non-reducing sugar is sucrose, trehalose, or rafinose. In a further embodiment, the sucrose is present at about 6% (w/v) to about 8% (w/v). In one embodiment, the sucrose is present at about 6% (w/v). In one embodiment, the sucrose is present at about 7% (w/v). In one embodiment, the sucrose is present at about 8% (w/v).

In certain embodiments, the anti-ILT4 formulations also comprise a surfactant. As used herein, a surfactant is a surface active agent that is amphipathic in nature. Surfactants can be added to the formulations herein to provide stability, reduce and/or prevent aggregation, or to prevent and/or inhibit protein damage during processing conditions such as purification, filtration, freeze-drying, transportation, storage, and delivery. In some embodiments, a surfactant can be useful for providing additional stability to the active ingredient(s).

Non-ionic surfactants that can be used in the formulations of the invention include, but are not limited to, polyoxyethylene sorbitan fatty acid esters (Polysorbates, sold under the trade name Tween® (Uniquema Americas LLC, Wilmington, DE)) including Polysorbate-20 (PS-20, polyoxyethylene sorbitan monolaurate), Polysorbate-40 (PS-40, polyoxyethylene sorbitan monopalmitate), Polysorbate-60 (PS-60, polyoxyethylene sorbitan monostearate), and Polysorbate-80 (PS-80, polyoxyethylene sorbitan monooleate); polyoxyethylene alkyl ethers such as Brij® 58 (Uniquema Americas LLC, Wilmington, DE) and Brij® 35; poloxamers (e.g., poloxamer 188); Triton® X-100 (Union Carbide Corp., Houston, TX) and Triton® X-114; NP40; Span 20, Span 40, Span 60, Span 65, Span 80 and Span 85; copolymers of ethylene and propylene glycol (e.g., the Pluronic® series of nonionic surfactants such as Pluronic® F68, Pluronic®10RS, Pluronic® F108, Pluronic® F127, Pluronic® F38, Pluronic® L44, Pluronic® L62 (BASF Corp., Ludwigshafen, Germany); and sodium dodecyl sulfate (SDS). In one embodiment, the non-ionic surfactant is PS-80 or PS-20. In one embodiment, the non-ionic surfactant is PS-20. In another embodiment, the non-ionic surfactant is PS-80.

The amount of non-ionic surfactant to be included in the formulations is an amount sufficient to perform the desired function, i.e., a minimal amount necessary to stabilize the active pharmaceutical ingredient (i.e., the anti-ILT4 antibody or antigen-binding fragment thereof (e.g., 1E1, 2A6, 3G7, or 2C1)) in the formulation. All percentages for the non-ionic surfactant are listed as % (w/v). Typically, the surfactant is present in a concentration of from about 0.008% to about 0.1% (w/v). In some embodiments of this aspect of the invention, the surfactant is present in the formulation in an amount from about 0.01% to about 0.1%, from about 0.01% to about 0.09%, from about 0.01% to about 0.08%, from about 0.01% to about 0.07%, from about 0.01% to about 0.06%, from about 0.01% to about 0.05%, from about 0.01% to about 0.04%, from about 0.01% to about 0.03%, from about 0.01% to about 0.02%, from about 0.015% to about 0.04%, from about 0.015% to about 0.03%, from about 0.015% to about 0.02%, from about 0.02% to about 0.04%, from about 0.02% to about 0.035%, or from about 0.02% to about 0.03%. In alternative embodiments, the surfactant is present in an amount of about 0.01%, about 0.015%, about 0.02%, about 0.025%, about 0.03%, about 0.035%, or about 0.04%.

In specific embodiments, the formulations described herein comprise about 0.01% to about 0.04% (w/v) PS-80. In one embodiment, the formulations described herein comprise PS-80 in an amount of about 0.01%. In one embodiment, the amount of PS-80 is about 0.015%. In another embodiment, the amount of PS-80 is about 0.02%. In a further embodiment, the amount of PS-80 is about 0.025%. In another embodiment, the amount of PS-80 is about 0.03%. In a further embodiment, the amount of PS-80 is about 0.035%. In another embodiment, the amount of PS-80 is about 0.04%. In a further embodiment, the amount of PS-80 is about 0.045%.

The formulations described herein also comprise methionine, or pharmaceutically acceptable salt thereof as an antioxidant. In one embodiment, the methionine is L-methionine. In another embodiment, the methionine is a pharmaceutically acceptable salt of L-methionine, such as, for example, methionine HCl. In an embodiment of the invention, methionine is present in the formulation at a concentration of about 1-20 mM (1, 2, 3, 4, 5, 6, 7, 8, 9, 10, 11, 12, 13, 14, 15, 16, 17, 18, 19, and 20 mM). In another embodiment, the methionine is present from about 5 mM to about 10 mM (5, 6, 7, 8, 9, and 10 mM). In another embodiment, the methionine is present at about 10 mM.

Thus, in one aspect, provided herein is a pharmaceutical formulation for an anti-ILT4 antibody or antigen-binding fragment thereof, comprising: (i) about 10 mg/mL to about 200 mg/mL of the anti-ILT4 antibody or antigen-binding fragment thereof; (ii) about 5 mM to about 20 mM of a buffer; (iii) about 6% to about 8% weight/volume (w/v) of a non-reducing sugar; (iv) about 0.01% to about 0.10% (w/v) of a non-ionic surfactant; and (v) about 1 mM to about 20 mM of an anti-oxidant.

In certain embodiments, the buffer is a selected from the group consisting of L-histidine buffer, acetate buffer, and citrate buffer. In one embodiment, the buffer is L-histidine buffer. In another embodiment, the buffer is acetate buffer. In yet another embodiment, the buffer is citrate buffer.

In some embodiments, the non-reducing sugar is sucrose.

In certain embodiments, the non-ionic surfactant is PS-80 or PS-20. In one embodiment, the non-ionic surfactant is PS-80. In another embodiment, the non-ionic surfactant is PS-20.

In some embodiments, the anti-oxidant is L-methionine.

Thus, in another aspect, provided herein is a pharmaceutical formulation for an anti-ILT4 antibody or antigen-binding fragment thereof, comprising: (i) about 10 mg/mL to about 200 mg/mL of the anti-ILT4 antibody or antigen-binding fragment thereof; (ii) about 5 mM to about 20 mM L-histidine buffer; (iii) about 6% to about 8% (w/v) sucrose; (iv) about 0.01% to about 0.10% (w/v) PS-80; and (v) about 1 mM to about 20 mM L-methionine.

In some embodiments, the formulation comprises about 8 mM to about 12 mM L-histidine buffer.

In certain embodiments, the formulation comprises about 5 mM to about 10 mM L-methionine.

In other embodiments, the formulation comprises about 0.01% to about 0.05% (w/v) PS-80.

In yet other embodiments, the formulation comprises about 10 mg/mL to about 150 mg/mL of the anti-ILT4 antibody or antigen-binding fragment thereof. In still other embodiments, the concentration of the anti-ILT4 antibody or antigen-binding fragment thereof is about 10 mg/mL, about 12.5 mg/mL, about 15 mg/mL, about 25 mg/mL, about 50 mg/mL, about 75 mg/mL, about 100 mg/mL, about 125 mg/mL, or about 150 mg/mL. In one embodiment, the concentration of the anti-ILT4 antibody or antigen-binding fragment thereof is about 10 mg/mL. In another embodiment, the concentration of the anti-ILT4 antibody or antigen-binding fragment thereof is about 12.5 mg/mL. In yet another embodiment, the concentration of the anti-ILT4 antibody or antigen-binding fragment thereof is about 15 mg/mL. In still another embodiment, the concentration of the anti-ILT4 antibody or antigen-binding fragment thereof is about 25 mg/mL. In one embodiment, the concentration of the anti-ILT4 antibody or antigen-binding fragment thereof is about 50 mg/mL. In another embodiment, the concentration of the anti-ILT4 antibody or antigen-binding fragment thereof is about 75 mg/mL. In yet another embodiment, the concentration of the anti-ILT4 antibody or antigen-binding fragment thereof is about 100 mg/mL. In still another embodiment, the concentration of the anti-ILT4 antibody or antigen-binding fragment thereof is about 125 mg/mL. In yet still another embodiment, the concentration of the anti-ILT4 antibody or antigen-binding fragment thereof is about 150 mg/mL.

Thus, in one specific embodiment, the formulation comprises about 25 mg/mL of the anti-ILT4 antibody or antigen-binding fragment thereof, about 10 mM L-histidine buffer, about 7% w/v sucrose, about 0.025% polysorbate 80, and about 10 mM L-methionine.

In another specific embodiment, the formulation comprises about 50 mg/mL of the anti-ILT4 antibody or antigen-binding fragment thereof, about 10 mM L-histidine buffer, about 7% w/v sucrose, about 0.025% polysorbate 80, and about 10 mM L-methionine.

In yet another specific embodiment, the formulation comprises about 75 mg/mL of the anti-ILT4 antibody or antigen-binding fragment thereof, about 10 mM L-histidine buffer, about 7% w/v sucrose, about 0.025% polysorbate 80, and about 10 mM L-methionine.

In still another specific embodiment, the formulation comprises about 100 mg/mL of the anti-ILT4 antibody or antigen-binding fragment thereof, about 10 mM L-histidine buffer, about 7% w/v sucrose, about 0.025% polysorbate 80, and about 10 mM L-methionine.

In yet still another specific embodiment, the formulation comprises about 125 mg/mL of the anti-ILT4 antibody or antigen-binding fragment thereof, about 10 mM L-histidine buffer, about 7% w/v sucrose, about 0.025% polysorbate 80, and about 10 mM L-methionine.

In certain embodiments of various formulations provided herein, the formulation has a pH of about 5.0-about 6.8. In some embodiments, the formulation has a pH of about 5.5-about 6.0. In other embodiments, the formulation has a pH of about 5.5, about 5.6, about 5.7, about 5.8, about 5.9, or about 6.0. In one embodiment, the formulation has a pH of about 5.5. In another embodiment, the formulation has a pH of about 5.6. In yet another embodiment, the formulation has a pH of about 5.7. In still another embodiment, the formulation has a pH of about 5.8. In another embodiment, the formulation has a pH of about 5.9. In yet still another embodiment, the formulation has a pH of about 6.0.

Thus, in one specific embodiment, the pharmaceutical formulation for an anti-ILT4 antibody or antigen-binding fragment thereof comprises: (i) about 50 mg/mL to about 100 mg/mL of the anti-ILT4 antibody or antigen-binding fragment thereof; (ii) about 10 mM L-histidine buffer, pH about 5.5; (iii) about 7% (w/v) sucrose; (iv) about 0.025% (w/v) polysorbate 80; and (v) about 10 mM L-methionine.

In certain embodiments of the various formulations disclosed herein, the anti-ILT4 antibody or antigen-binding fragment thereof is an anti-ILT4 antibody or antigen-binding fragment thereof described herein (e.g., 1E1, 2A6, 3G7, or 2C1). In some embodiments of the formulations, the anti-ILT4 antibody or antigen-binding fragment thereof is 1E1 or a variant thereof. In some embodiments of the formulations, the anti-ILT4 antibody or antigen-binding fragment thereof is 2A6 or a variant thereof. In some embodiments of the formulations, the anti-ILT4 antibody or antigen-binding fragment thereof is 3G7 or a variant thereof. In some embodiments of the formulations, the anti-ILT4 antibody or antigen-binding fragment thereof is 2C1 or a variant thereof.

In certain embodiments of the various formulations provided herein, the anti-ILT4 antibody or antigen binding fragment thereof comprises a heavy chain variable domain comprising: CDR-H1: GYYWS (SEQ ID NO:16), CDR-H2: EINHXGSTNYNPSLKS wherein X is S or A (SEQ ID NO:17), and CDR-H3: LPTRWVTTRYFDL (SEQ ID NO:18); and a light chain variable domain comprising: CDR-L1: TGSSSNIGAGYDVH (SEQ ID NO:19), CDR-L2: GX₁X₂NRPS, wherein X₁ is N, Q, E or D and X₂ is S or A (SEQ ID NO:20), and CDR-L3: QSFDNSLSAYV (SEQ ID NO:21).

In some embodiments of the various formulations provided herein, the anti-ILT4 antibody or antigen binding fragment thereof comprises a heavy chain variable domain comprising: CDR-H1: GYYWS (SEQ ID NO:16), CDR-H2: EINHAGSTNYNPSLKS (SEQ ID NO:48), and CDR-H3: LPTRWVTTRYFDL (SEQ ID NO:18); and a light chain variable domain comprising: CDR-L1: TGSSSNIGAGYDVH (SEQ ID NO:19), CDR-L2: GDSNRPS (SEQ ID NO:52), and CDR-L3: QSFDNSLSAYV (SEQ ID NO:21).

In other embodiments of the various formulations provided herein, the anti-ILT4 antibody or antigen binding fragment thereof comprises a heavy chain variable domain comprising the amino acid sequence set forth in SEQ ID NO:57 and a light chain variable domain comprising the amino acid sequence set forth in SEQ ID NO:58.

In yet other embodiments of the various formulations provided herein, the anti-ILT4 antibody or antigen binding fragment thereof comprises a heavy chain comprising the amino acid sequence set forth in SEQ ID NO:2 and a light chain comprising the amino acid sequence set forth in SEQ ID NO:7.

In still other embodiments of the various formulations provided herein, the anti-ILT4 antibody or antigen binding fragment thereof comprises a heavy chain comprising the amino acid sequence set forth in SEQ ID NO:80 and a light chain comprising the amino acid sequence set forth in SEQ ID NO:7.

In certain embodiments of the various formulations provided herein, the anti-ILT4 antibody or antigen binding fragment thereof comprises a heavy chain variable domain consisting of the amino acid sequence set forth in SEQ ID NO:57 and a light chain variable domain consisting of the amino acid sequence set forth in SEQ ID NO:58.

In some embodiments of the various formulations provided herein, the anti-ILT4 antibody or antigen binding fragment thereof comprises a heavy chain consisting of the amino acid sequence set forth in SEQ ID NO:2 and a light chain consisting of the amino acid sequence set forth in SEQ ID NO:7.

In other embodiments of the various formulations provided herein, the anti-ILT4 antibody or antigen binding fragment thereof comprises a heavy chain consisting of the amino acid sequence set forth in SEQ ID NO:80 and a light chain consisting of the amino acid sequence set forth in SEQ ID NO:7.

Thus, in some embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain variable domain comprising: CDR-H1: GYYWS (SEQ ID NO:16), CDR-H2: EINHXGSTNYNPSLKS wherein X is S or A (SEQ ID NO:17), and CDR-H3: LPTRWVTTRYFDL (SEQ ID NO:18); and a light chain variable domain comprising: CDR-L1: TGSSSNIGAGYDVH (SEQ ID NO:19), CDR-L2: GX₁X₂NRPS, wherein X₁ is N, Q, E or D and X₂ is S or A (SEQ ID NO:20), and CDR-L3: QSFDNSLSAYV (SEQ ID NO:21).

In certain embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain variable domain comprising: CDR-H1: GYYWS (SEQ ID NO:16), CDR-H2: EINHAGSTNYNPSLKS (SEQ ID NO:48), and CDR-H3: LPTRWVTTRYFDL (SEQ ID NO:18); and a light chain variable domain comprising: CDR-L1: TGSSSNIGAGYDVH (SEQ ID NO:19), CDR-L2: GDSNRPS (SEQ ID NO:52), and CDR-L3: QSFDNSLSAYV (SEQ ID NO:21).

In other embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain variable domain comprising the amino acid sequence set forth in SEQ ID NO:57 and a light chain variable domain comprising the amino acid sequence set forth in SEQ ID NO:58.

In yet other embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain comprising the amino acid sequence set forth in SEQ ID NO:2 and a light chain comprising the amino acid sequence set forth in SEQ ID NO:7.

In still other embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain comprising the amino acid sequence set forth in SEQ ID NO:80 and a light chain comprising the amino acid sequence set forth in SEQ ID NO:7.

In some embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain variable domain consisting of the amino acid sequence set forth in SEQ ID NO:57 and a light chain variable domain consisting of the amino acid sequence set forth in SEQ ID NO:58.

In some embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain consisting of the amino acid sequence set forth in SEQ ID NO:2 and a light chain consisting of the amino acid sequence set forth in SEQ ID NO:7.

In some embodiments, provided herein is a pharmaceutical formulation for an anti-ILT4 antibody or antigen-binding fragment thereof, comprising: (i) about 25 mg/mL to about 100 mg/mL of the anti-ILT4 antibody or antigen-binding fragment thereof; (ii) about 10 mM L-histidine buffer; (iii) about 7% (w/v) sucrose; (iv) about 0.025% (w/v) PS-80; and (v) about 10 mM L-methionine; wherein the anti-ILT4 antibody or antigen binding fragment thereof comprises a heavy chain consisting of the amino acid sequence set forth in SEQ ID NO:80 and a light chain consisting of the amino acid sequence set forth in SEQ ID NO:7.

In one embodiment, the formulation is a liquid formulation. In another embodiment, the liquid formulation is stored at about 3° C.-5° C. In yet another embodiment, the liquid formulation is frozen to at least −70° C. or below. In still another embodiment, the liquid formulation is a reconstituted solution from a lyophilized formulation.

In certain embodiments, after the formulation is stored at about 3° C. to about 5° C. for 6 months, (i) the % monomer of the anti-ILT4 antibody or antigen-binding fragment thereof is at least about 99% as determined by ultra-performance size exclusion chromatography; (ii) the turbidity of the formulation is at most about 0.135 as measured by OD₃₅₀₋₅₀₀; (iii) the % main peak of the anti-ILT4 antibody or antigen-binding fragment thereof is at least about 63%, the % acidic variant of the anti-ILT4 antibody or antigen-binding fragment thereof is at most about 23%, and/or the basic variant of the anti-ILT4 antibody or antigen-binding fragment thereof is at most about 14%, as determined by high performance ion-exchange chromatography; (iv) the subvisible particle count of particles that are at least 2 μm in size is at most about 3500 as determined by microflow imaging; and/or (v) the % oxidation of one or more amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping.

in some embodiments, after the formulation is stored at about 3° C. to about 5° C. for 6 months, the % monomer of the anti-ILT4 antibody or antigen-binding fragment thereof is at least about 99% as determined by ultra-performance size exclusion chromatography.

In some embodiments, after the formulation is stored at about 3° C. to about 5° C. for 6 months, the turbidity of the formulation is at most about 0.135 as measured by OD₃₅₀₋₅₀₀.

In some embodiments, after the formulation is stored at about 3° C. to about 5° C. for 6 months, the % main peak of the anti-ILT4 antibody or antigen-binding fragment thereof is at least about 63%, as determined by high performance ion-exchange chromatography. In some embodiments, after the formulation is stored at about 3° C. to about 5° C. for 6 months, the % acidic variant of the anti-ILT4 antibody or antigen-binding fragment thereof is at most about 23%, as determined by high performance ion-exchange chromatography. In some embodiments, after the formulation is stored at about 3° C. to about 5° C. for 6 months, the basic variant of the anti-ILT4 antibody or antigen-binding fragment thereof is at most about 14%, as determined by high performance ion-exchange chromatography. In some embodiments, after the formulation is stored at about 3° C. to about 5° C. for 6 months, the % main peak of the anti-ILT4 antibody or antigen-binding fragment thereof is at least about 63%, the % acidic variant of the anti-ILT4 antibody or antigen-binding fragment thereof is at most about 23%, and the basic variant of the anti-ILT4 antibody or antigen-binding fragment thereof is at most about 14%, as determined by high performance ion-exchange chromatography.

In some embodiments, after the formulation is stored at about 3° C. to about 5° C. for 6 months, the subvisible particle count of particles that are at least 2 μm in size is at most about 3500 as determined by microflow imaging.

In some embodiments, after the formulation is stored at about 3° C. to about 5° C. for 6 months, the % oxidation of one or more amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of one amino acid residue selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of two amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of three amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of four amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping. In one embodiment, after the formulation is stored at about 3° C. to about 5° C. for 6 months, the % oxidation of all five amino acid residues from the group consisting of W7, W102, M253, M359, and M429 in the heavy chain of the anti-ILT4 antibody as set forth in SEQ ID NO:2 or 80 is less than about 4%, as determined by reduced peptide mapping.

The formulations described herein can be made as a liquid formulation. A liquid antibody formulation can be made by taking the drug substance (e.g., anti-ILT4 monoclonal antibody) which is in liquid form (e.g., anti-ILT4 monoclonal antibody in an aqueous pharmaceutical formulation) and buffer exchanging it into the desired buffer as the last step of the purification process. There is no lyophilization step in this embodiment. The drug substance in the final buffer is concentrated to a desired concentration. Excipients such as sucrose and PS80 are added to the drug substance and it is diluted using the appropriate buffer to final protein concentration. The final formulated drug substance is filtered using 0.22 m filters and filled into a final container (e.g., glass vials).

The formulations described herein can also be reconstituted from lyophilized formulations. Lyophilized formulations of therapeutic proteins provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e., in a small volume) if necessary for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically, the lyophilized formulation is prepared in anticipation of reconstitution at high concentration of drug product (DP, in an exemplary embodiment anti-ILT4 antibody or antigen-binding fragment thereof), i.e., in anticipation of reconstitution in a low volume of water. Subsequent dilution with water or isotonic buffer can then readily be used to dilute the DP to a lower concentration. Typically, excipients are included in a lyophilized formulation of the invention at levels that will result in a roughly isotonic formulation when reconstituted at high DP concentration, e.g., for subcutaneous administration. Reconstitution in a larger volume of water to give a lower DP concentration will necessarily reduce the tonicity of the reconstituted solution, but such reduction may be of little significance in non-subcutaneous, e.g., intravenous, administration. If isotonicity is desired at lower DP concentration, the lyophilized powder may be reconstituted in the standard low volume of water and then further diluted with isotonic diluent, such as 0.9% sodium chloride.

The lyophilized formulations of the invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g., 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations are reconstituted prior to administration. The protein may be reconstituted at a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. In one embodiment, the protein concentration after reconstitution is about 10-300 mg/mL. In one embodiment, the protein concentration after reconstitution is about 20-250 mg/mL. In one embodiment, the protein concentration after reconstitution is about 150-250 mg/mL. In one embodiment, the protein concentration after reconstitution is about 180-220 mg/mL. In one embodiment, the protein concentration after reconstitution is about 50-150 mg/mL. In one embodiment, the protein concentration after reconstitution is about 150 mg/mL. In one embodiment, the protein concentration after reconstitution is about 125 mg/mL. In one embodiment, the protein concentration after reconstitution is about 100 mg/mL. In one embodiment, the protein concentration after reconstitution is about 75 mg/mL. In one embodiment, the protein concentration after reconstitution is about 50 mg/mL. In one embodiment, the protein concentration after reconstitution is about 25 mg/mL. High protein concentrations are particularly useful where subcutaneous delivery of the reconstituted formulation is intended. However, for other routes of administration, such as intravenous administration, lower concentrations of the protein may be desired (e.g., from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g., phosphate-buffered saline), sterile saline solution, Ringer's solution or dextrose solution.

### Methods of Use

In another aspect, provided herein is a method of treating cancer in a subject, comprising administering a therapeutically effective amount of any formulation described herein to the subject. In some specific embodiments of this method, the formulation is administered to the subject via intravenous administration. In other embodiments, the formulation is administered to the subject by subcutaneous administration. In one embodiment, the invention comprises a method of treating cancer in a human patient comprising administering any formulation described herein to the patient.

In any of the methods of the invention, the cancer can be selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, salivary cancer, prostate cancer (e.g., hormone refractory prostate adenocarcinoma), pancreatic cancer, colon cancer, esophageal cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is mediastinal large B-cell lymphoma.

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In some embodiments, the cancer is a solid tumor with a high level of microsatellite instability (MSI-H).

In some embodiments, the cancer is a solid tumor with a high mutational burden.

In some embodiments, the cancer is metastatic colorectal cancer with high levels of microsatellite instability (MSI-H).

In some embodiments, the cancer is selected from the group consisting of colorectal cancer, esophageal cancer, melanoma, non-small cell lung cancer, ovarian cancer, renal cell cancer and small cell lung cancer.

In other embodiments of the above treatment methods, the cancer is a hematological malignancy. In certain embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mci-1), myelodysplastic syndrome (MDS), non-Hodgkin lymphoma (NHL), or small lymphocytic lymphoma (SLL).

Malignancies that demonstrate improved disease-free and overall survival in relation to the presence of tumor-infiltrating lymphocytes in biopsy or surgical material, e.g., melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

Additional cancers that can benefit from treatment with the formulations described herein include those associated with persistent infection with viruses such as human immunodeficiency viruses, hepatitis viruses class A, B and C, Epstein Barr virus, human papilloma viruses that are known to be causally related to for instance Kaposi's sarcoma, liver cancer, nasopharyngeal cancer, lymphoma, cervical, vulval, anal, penile and oral cancers.

The formulations can also be used to prevent or treat infections and infectious disease. Thus, the invention provides a method for treating chronic infection in a mammalian subject comprising administering an effective amount of a formulation of the invention to the subject. In some specific embodiments of this method, the formulation is administered to the subject via intravenous administration. In other embodiments, the formulation is administered to the subject by subcutaneous administration.

These agents can be used alone, or in combination with vaccines, to stimulate the immune response to pathogens, toxins, and self-antigens. The antibodies or antigen-binding fragment thereof can be used to stimulate immune response to viruses infectious to humans, including but not limited to: human immunodeficiency viruses, hepatitis viruses class A, B and C, Epstein Barr virus, human cytomegalovirus, human papilloma viruses, and herpes viruses. Antagonist anti-PD-1 antibodies or antibody fragments can be used to stimulate immune response to infection with bacterial or fungal parasites, and other pathogens. Viral infections with hepatitis B and C and HIV are among those considered to be chronic viral infections.

The formulations of the invention may be administered to a patient in combination with one or more “additional therapeutic agents”. The additional therapeutic agent may be a biotherapeutic agent (including but not limited to antibodies to VEGF, EGFR, Her2/neu, VEGF receptors, other growth factor receptors, CD20, CD40, CD-40L, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-PD-1 antibody or antigen-binding fragment thereof, an anti-PD-L1 antibody or antigen binding fragment thereof, an anti-ILT4 antibody or antigen binding fragment thereof, an anti-ILT4 antibody or antigen binding fragment thereof, an anti-GITR antibody or antigen-binding fragment thereof, an anti-CTL4 antibody or antigen-binding fragment thereof, an anti-CD27 antibody or antigen-binding fragment thereof. In one embodiment, the additional therapeutic agent is a Newcastle disease viral vector expressing IL-12. In a further embodiment, the additional therapeutic agent is dinaciclib. In still further embodiments, the additional therapeutic agent is a STING agonist.

Suitable routes of administration may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g., subcutaneous administration) require a lyophilized formulation to enable reconstitution at high concentration.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g., biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See. e.g., Wawrzynczak (1996) Antibody Therapy, Bios Scientific Pub. Ltd, Oxfordshire, UK; Kresina (ed.) (1991) Monoclonal Antibodies. Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See. e.g., Remington's Pharmaceutical Sciences and U.S. Pharmacopeia: National Formulary, Mack Publishing Company, Easton, PA (1984); Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY: Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY.

A pharmaceutical antibody formulation can be administered by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

Embodiments of the invention also include one or more of the biological formulations described herein (i) for use in, (ii) for use as a medicament or composition for, or (iii) for use in the preparation of a medicament for: (a) therapy (e.g., of the human body); (b) medicine; (c) induction of or increasing of an antitumor immune response; (d) decreasing the number of one or more tumor markers in a patient; (e) halting or delaying the growth of a tumor or a blood cancer; (f) halting or delaying the progression of a PD-1-related disease or an ILT4-related disease; (g) stabilization of a PD-1-related disease or an ILT4-related disease: (h) inhibiting the growth or survival of tumor cells; (i) eliminating or reducing the size of one or more cancerous lesions or tumors; (j) reduction of the progression, onset or severity of a PD-1-related disease or an ILT4-related disease: (k) reducing the severity or duration of the clinical symptoms of a PD-1-related or ILT4-related disease; (1) prolonging the survival of a patient relative to the expected survival in a similar untreated patient; (m) inducing complete or partial remission of a cancerous condition or other PD-1-related or ILT4-related disease; (n) treatment of cancer; or (o) treatment of chronic infections.

### General Methods

Standard methods in molecular biology are described in Sambrook, Fritsch and Maniatis (1982 & 1989 2^{nd} Edition, 2001 3^{nd} Edition) Molecular Cloning, A Laboratory Manual, Cold Spring Harbor Laboratory Press, Cold Spring Harbor, NY: Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA. Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (200) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry. 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTIS) Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, el al. (2002) Comput. Methods Programs Biomed. 68:177-181: von Heijne (1983) Eur. J. Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetry (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the α-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbent assay).

All publications mentioned herein are incorporated by reference for the purpose of describing and disclosing methodologies and materials that might be used in connection with the invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

Embodiment 1 provides a formulation comprising: (i) about 10 mg/mL to about 200 mg/mL of an anti-human immunoglobulin-like transcript 4 (“anti-ILT4”) antibody or antigen-binding fragment thereof; (ii) about 10 mg/mL to about 200 mg/mL of an anti-PD-1 antibody or antigen-binding fragment thereof; (iii) about 5 mM to about 20 mM of a buffer, (iv) about 6% to about 8% weight/volume (w/v) of a non-reducing sugar; (v) about 0.01% to about 0.10% (w/v) of a non-ionic surfactant; and (vi) about 1 mM to about 20 mM of an anti-oxidant, wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21), wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 102, wherein the buffer is an L-histidine buffer, an acetate buffer, or a citrate buffer, wherein the non-reducing sugar is a disaccharide, wherein the non-ionic surfactant is polysorbate 20 or polysorbate 80, and wherein the antioxidant is methionine or a pharmaceutically acceptable salt thereof.

Embodiment 2 provides the formulation of embodiment 1, comprising 40-60 mg/mL of the anti-ILT4 antibody; 15-35 mg/mL of the anti-PD-1 antibody; about 7% sucrose or trehalose; about 0.025% polysorbate 80 or polysorbate 20; about 3-30 mM L-histidine buffer at pH about 5.0-6.5; and about 5-20 mM methionine or a pharmaceutically acceptable salt thereof.

Embodiment 3 provides the formulation of embodiment 1 comprising 45-55 mg/mL of the anti-ILT4 antibody; 20-30 mg/mL of the anti-PD-1 antibody; about 7% sucrose or trehalose; about 0.025% polysorbate 80 or polysorbate 20; about 3-30 mM L-histidine buffer at pH about 5.0-6.5; and about 10-20 mM L-methionine or a pharmaceutically acceptable salt thereof.

Embodiment 4 provides the formulation of embodiment 1 wherein the buffer is an L-histidine buffer.

Embodiment 5 provides a formulation of any of embodiments 1-4, wherein the non-reducing sugar is sucrose.

Embodiment 6 provides a formulation of any one of embodiments 1-5, wherein the non-ionic surfactant is polysorbate 80.

Embodiment 7 provides a formulation of any one of embodiments 1-6, wherein the anti-oxidant is L-methionine.

Embodiment 8 provides a formulation comprising: (i) about 10 mg/mL to about 200 mg/mL of an anti-ILT4 antibody (“anti-ILT4”) or antigen-binding fragment thereof; (ii) about 10 mg/mL to about 200 mg/mL of an anti-PD-1 antibody or antigen-binding fragment thereof; (iii) about 5 mM to about 20 mM L-histidine buffer; (iv) about 6% to about 8% weight/volume (w/v) sucrose; (v) about 0.01% to about 0.10% (w/v) polysorbate 80; and (vi) about 1 mM to about 20 mM L-methionine, wherein the anti-ILT4 antibody or antigen binding fragment thereof comprises a heavy chain variable domain comprising: CDR-HL: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21); and wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 102, or a pharmaceutically acceptable salt thereof.

Embodiment 9 provides a formulation of embodiment 8, comprising about 8 mM to about 12 mM L-histidine buffer.

Embodiment 10 provides a formulation of any one of embodiments f 8 or 9, comprising about 5 mM to about 10 mM L-methionine.

Embodiment 11 provides a formulation of any one of embodiments 8-10, comprising about 0.01% to about 0.05% (w/v) polysorbate 80.

Embodiment 12 provides a formulation of any one of embodiments 1 or 4-11, comprising about 10 mg/mL to about 150 mg/mL of the anti-ILT4 antibody or antigen-binding fragment thereof.

Embodiment 13 provides a formulation of any one of embodiments 1 or 4-11, comprising about 10 mg/mL to about 150 mg/mL of the anti-PD-1 antibody or antigen-binding fragment thereof.

Embodiment 14 provides a formulation of any one of embodiments 12, wherein the concentration of the anti-ILT4 antibody or antigen-binding fragment thereof is about 10 mg/mL, about 12.5 mg/mL, about 25 mg/mL, about 50 mg/mL, about 75 mg/mL, about 100 mg/mL, about 125 mg/mL, or about 150 mg/mL.

Embodiment 15 provides a formulation of any one of embodiments 12, wherein the concentration of the anti-PD-1 antibody or antigen-binding fragment thereof is about 10 mg/mL, about 12.5 mg/mL, about 25 mg/mL, about 50 mg/mL, about 75 mg/mL, about 100 mg/mL, about 125 mg/mL, or about 150 mg/mL.

Embodiment 16 provides a formulation of any one of embodiments 1-13, comprising about 25 mg/mL of the anti-ILT4 antibody or antigen-binding fragment thereof, about 10 mg/mL of the anti-PD-1 antibody or antigen binding fragment thereof, about 10 mM L-histidine buffer, about 7% w/v sucrose, about 0.025% polysorbate 80, and about 10 mM L-methionine.

Embodiment 17 provides a formulation of any one of embodiments 1-13, comprising about 50 mg/mL of the anti-ILT4 antibody or antigen-binding fragment thereof, about 25 mg/mL of the anti-PD-1 antibody or antigen binding fragment thereof, about 10 mM L-histidine buffer, about 7% w/v sucrose, about 0.025% polysorbate 80, and about 10 mM L-methionine.

Embodiment 18 provides a formulation of any one of embodiments 1-13, comprising about 75 mg/mL of the anti-ILT4 antibody or antigen-binding fragment thereof, about 50 mg/mL of the anti-PD-1 antibody or antigen binding fragment thereof, about 10 mM L-histidine buffer, about 7% w/v sucrose, about 0.025% polysorbate 80, and about 10 mM L-methionine.

Embodiment 19 provides a formulation of any one of embodiments 1-13, comprising about 100 mg/mL of the anti-ILT4 antibody or antigen-binding fragment thereof, about 75 mg/mL of the anti-PD-1 antibody or antigen binding fragment thereof, about 10 mM L-histidine buffer, about 7% w/v sucrose, about 0.025% polysorbate 80, and about 10 mM L-methionine.

Embodiment 20 provides a formulation of any one of embodiments 1-13, comprising about 125 mg/mL of the anti-ILT4 antibody or antigen-binding fragment thereof, about 100 mg/mL of the anti-PD-1 antibody or antigen binding fragment thereof, about 10 mM L-histidine buffer, about 7% w/v sucrose, about 0.025% polysorbate 80, and about 10 mM L-methionine.

Embodiment 21 provides a formulation of any one of embodiments 1-20, wherein the formulation has a pH of about 5.0 to about 6.8.

Embodiment 22 provides a formulation of any one of embodiments 1-21, wherein the formulation has a pH of about 5.5 to about 6.0.

Embodiment 23 provides a formulation of any one of embodiments 1-22, wherein the formulation has a pH of about 5.5.

Embodiment 24 provides a formulation of an anti-human immunoglobulin-like transcript 4 (“anti-ILT4”) antibody or antigen-binding fragment thereof, comprising: (i) about 50 mg/mL to about 100 mg/mL of the anti-ILT4 antibody or antigen-binding fragment thereof; (ii) about 10 mg/mL to about 200 mg/mL of an anti-PD-1 antibody or antigen-binding fragment thereof; (iii) about 10 mM L-histidine buffer, pH about 5.5; (iv) about 7% weight/volume (w/v) sucrose; (v) about 0.025% (w/v) polysorbate 80; and (vi) about 10 mM L-methionine, wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21), and wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 102.

Embodiment 25 provides a formulation of any one of embodiments 1-24, wherein the anti-ILT4 antibody or antigen binding fragment thereof comprises a heavy chain variable domain comprising: CDR-H1: GYYWS (SEQ ID NO: 16), CDR-H2: EINHSGSTNYNPSLKS (SEQ ID NO:47), and CDR-H3: LPTRWVITRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21).

Embodiment 26 provides a formulation of any one of embodiments 1-24, wherein the anti-ILT4 antibody or antigen binding fragment thereof comprises a heavy chain variable domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GDSNRPS (SEQ ID NO: 52), and CDR-L3: QSFDNSLSAYV (SEQ ID NO: 21).

Embodiment 27 provides a formulation of any one of embodiments 1-24, wherein the anti-ILT4 antibody or antigen binding fragment thereof comprises a heavy chain variable domain comprising the amino acid sequence set forth in SEQ ID NO:57 and a light chain variable domain comprising the amino acid sequence set forth in SEQ ID NO:58.

Embodiment 28 provides a formulation of any one of embodiments 1-24, wherein the anti-ILT4 antibody or antigen binding fragment thereof comprises a heavy chain comprising the amino acid sequence set forth in SEQ ID NO:2 and a light chain comprising the amino acid sequence set forth in SEQ ID NO:7.

Embodiment 29 provides a formulation of any one of embodiments 1-24, wherein the anti-ILT4 antibody or antigen binding fragment thereof comprises a heavy chain comprising the amino acid sequence set forth in SEQ ID NO:80 and a light chain comprising the amino acid sequence set forth in SEQ ID NO:7.

Embodiment 30 provides a formulation of any one of embodiments 1-24, wherein the anti-ILT4 antibody or antigen binding fragment thereof comprises a heavy chain variable domain consisting of the amino acid sequence set forth in SEQ ID NO:57 and a light chain variable domain consisting of the amino acid sequence set forth in SEQ ID NO:58.

Embodiment 31 provides a formulation of any one of embodiments 1-24, wherein the anti-ILT4 antibody or antigen binding fragment thereof comprises a heavy chain consisting of the amino acid sequence set forth in SEQ ID NO:2 and a light chain consisting of the amino acid sequence set forth in SEQ ID NO:7.

Embodiment 32 provides a formulation of any one of embodiments 1-24, wherein the anti-ILT4 antibody or antigen binding fragment thereof comprises a heavy chain consisting of the amino acid sequence set forth in SEQ ID NO:80 and a light chain consisting of the amino acid sequence set forth in SEQ ID NO:7.

Embodiment 33 provides a formulation of any one of embodiments 1-24, wherein the anti-human PD-1 antibody or antigen binding fragment thereof comprises a variable light region which comprises the amino acid sequence set forth in SEQ ID NO: 98, and a variable heavy region which comprises the amino acid sequence set forth in SEQ ID NO: 103.

Embodiment 34 provides a formulation of any one of embodiments 1-24, wherein the anti-human PD-1 antibody or antigen binding fragment thereof comprises a variable light region which comprises the amino acid sequence set forth in SEQ ID NO: 99, and a variable heavy region which comprises the amino acid sequence set forth in SEQ ID NO: 104.

Embodiment 35 provides a formulation of any one of embodiments 1-24, wherein the anti-human PD-1 antibody or antigen binding fragment thereof consists of two light chains and two heavy chains, wherein the two light chains consist of the amino acid sequence set forth in SEQ ID NO:99 and the two heavy chains consist of the amino acid sequence set forth in any one of SEQ ID NOs:104-109.

Embodiment 37 provides a formulation of any one of embodiments 1-35, wherein the anti-human PD-1 antibody is pembrolizumab.

Embodiment 37 provides a formulation of any one of embodiments 1-36, wherein the ratio of anti-ILT4 antibody to the anti-PD-1 antibody is 1:1 or 1:2.

Embodiment 38 provides a formulation of any one of embodiments 1-37, wherein after 9 months at 5° C.: (i) the % monomer of the anti-ILT4 antibody is ≥95% as determined by size exclusion chromatography; (ii) the % heavy chain and light chain of the anti-ILT4 antibody is 2 90% as measured by reduced CE-SDS; (iii) the % heavy chain and light chain of the anti-ILT4 antibody is 95% as measured by reduced CE-SDS; (iv) the % intact IgG of the anti-ILT4 antibody is 90% as measured by non-reduced CE-SDS; and/or (v) the % intact IgG of the anti-ILT4 antibody is ≥95% as measured by non-reduced CE-SDS.

Embodiment 39 provides a formulation of any one of embodiments 1-38, wherein the formulation is a liquid formulation stored at about 3° C. to about 5° C., or is a reconstituted solution from a lyophilized formulation.

Embodiment 40 provides a formulation of any one of embodiments 1-24, wherein after storage at about 3° C. to about 5° C. for up to 9 months: (i) the % monomer of the anti-ILT4 antibody or antigen-binding fragment thereof is at least about 99% as determined by ultra-performance size exclusion chromatography; (ii) the turbidity of the formulation is at most about 0.135 as measured by OD350-500; (iii) the % main peak of the anti-ILT4 antibody or antigen-binding fragment thereof is at least about 63%, the % acidic variant of the anti-ILT4 antibody or antigen-binding fragment thereof is at most about 23%, and/or the basic variant of the anti-ILT4 antibody or antigen-binding fragment thereof is at most about 14%, as determined by high performance ion-exchange chromatography; (iv) the subvisible particle count of particles that are at least 2 Om in size is at most about 3500 as determined by microflow imaging; and/or (v) the % oxidation of one or more amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping.

Embodiment 41 provides the formulation of any of embodiments 1-34 or 37-41, wherein the anti-PD-1 antibody or antigen binding fragment thereof is a monoclonal antibody.

Embodiment 42 provides a method of treating a cancer in a human patient in need thereof, the method comprising administering an effective amount of the formulation of any one of embodiments 1-40.

Embodiment 43 provides the method of embodiment 42, wherein the cancer is selected from the group consisting of colorectal cancer, esophageal cancer, melanoma, non-small cell lung cancer, ovarian cancer, renal cell cancer, and small cell lung cancer.

Embodiment 44 provides the use of the formulation of any one of embodiments 1-41 for preparing a medicament for treating a cancer.

Embodiment 45 provides the use of the formulation of any of one of embodiments 1-42 for the treatment of cancer in a human patient.

Embodiment 46 provides the use of embodiment 45, wherein the cancer is selected from the group consisting of colorectal cancer, esophageal cancer, melanoma, non-small cell lung cancer, ovarian cancer, renal cell cancer, and small cell lung cancer.

### EXAMPLES

### Example 1: Preparation of Anti-ILT4/Anti-PD-1 Co-Formulation

An anti-ILT4 monoclonal antibodies and anti-PD-1 antibodies (“anti-ILT4/anti-PD-1 co-formulation”) injection was prepared as a sterile, preservative-free solution that required dilution for intravenous infusion. The anti-ILT4/anti-PD-1 coformulation is a fixed dose combination of anti-ILT4 and pembrolizumab antibodies. The co-formulation composition was manufactured by combining fully formulated anti-ILT4 monoclonal antibody Process 2 drug substance and pembrolizumab drug substance. The anti-ILT4 drug substance contained 50.0 mg/mL anti-ILT4 in 0.29 mg/mL L-Histidine, 1.71 mg/mL L-Histidine Monohydrochloride Monohydrate, 1.49 mg/mL L-Methionine, 70.0 mg/mL Sucrose, and 0.25 mg/mL Polysorbate 80, pH 5.5. The pembrolizumab drug substance contained 25.0 mg/mL pembrolizumab in 0.29 mg/mL L-Histidine, 1.71 mg/mL L-Histidine Monohydrochloride Monohydrate, 70 mg/mL Sucrose, and 0.20 mg/mL Polysorbate 80, pH 5.5. The following formulation attributes were considered during formulation optimization: protein concentration, buffer, pH, charged species, antioxidant, and surfactant.

### Example 2: Pre Anti-ILT4/Pembrolizumab Co-Formulation Analysis

Table 6 summarizes the pre-coformulation development plan. The study was conducted to evaluate the sensitivity of anti-ILT4 drug product (DP), Single Entity (SE) and Co-Formulation (CF) with pembrolizumab, to different formulation and process parameters such as light exposure, oxygen, and residual levels of peroxide and metals that a product experiences during manufacturing and have previously known to impact oxidation. Filtered bulk DP of anti-ILT4 (30 mg/mL), pembrolizumab (7.5 mg/mL) and anti-ILT4+pembrolizumab (30 mg/mL+7.5 mg/mL) was spiked with residual levels of peroxide (0, 20 and 200 ppb), metals (0, 25 and 250 ppb), and subjected to light stress (0, 0.2× and 0.4×ICH of combined CWL+UV, where 1×ICH exposure level corresponds to 1.2 million lux hours of white light and 200 watt hours/m² of UV energy) followed by staging on stability up to 12 weeks at 5° C. and 4 weeks at 40° C. to assess the potential oxidative liability of anti-ILT4. The range of tested stress levels for metal, peroxide and light exposure were selected to cover the target exposures during DP manufacturing at a commercial manufacturing site and then bracketed appropriately for controls and worse-case scenarios. Additionally, this study also served for ranging free L-Methionine concentration (7, 10 and 15 mM), as an excipient for controlling protein oxidation. Samples were analyzed by HIC, IEX, SEC, L-Methionine content, PS-80 content, Sub-Visible Particles and Reduced Peptide Mapping assays.

The composition of anti-ILT4/anti-PD-1 co-formulation tested in this study is presented in Table 6. L-Methionine concentration (7, 10 and 15 mM), was ranged as an excipient for controlling protein oxidation

TABLE 6
Composition of anti-ILT4/anti-PD-1 CF, anti-ILT4 SE and pembrolizumab SE

| | | | Concentration (mg/mL) | | | |
|---|---|---|---|---|---|---|
| | | | anti- | | | |
| | | | ILT4/ | anti- | | |
| | | | anti-PD- | ILT4 | pembrolizumab | |
| Component | Grade | Function | 1 CF | SE | SE | Diluent |
| anti-ILT4 | In-house | Active | 30.0 | 30.0 | 0 | 0 |
| monoclonal | specification | pharmaceutical | | | | |
| antibody | | ingredient | | | | |
| pembrolizumab | In-house | Active | 7.5 | 0 | 7.5 | 0 |
| | specification | pharmaceutical | | | | |
| | | ingredient | | | | |
| L-Histidine | USP or Ph. | Buffer | 0.56 | 0.56 | 0.56 | 0.56 |
| | Eur. or JP | | | | | |
| L-Histidine | Ph. Eur. or BP | Buffer | 1.35 | 1.35 | 1.35 | 1.35 |
| Monohydrochloride | or JP | | | | | |
| Monohydrate | | | | | | |
| Sucrose | Ph. Eur. or BP | Stabilizer/ | 70.0 | 70.0 | 70.0 | 70.0 |
| | or JP or NF | Tonicity | | | | |
| | | Modifier | | | | |
| Polysorbate 80 | USP or NF or | Surfactant | 0.25 | 0.25 | 0.25 | 0.25 |
| | Ph. Eur. or JP | | | | | |
| Water for | USP or Ph. | Solvent | q.s. to 1.0 mL | | | |
| Injection | Eur. | | | | | |

q.s. = quantum sufficit, the quantity that is sufficient
L-Methionine concentration was ranged as part of the DOE study design

### Example 3: Evaluation of Oxidation Sensitivity

The five critical parameters studied in the study were L-Met, peroxide, metal, filling volume and light stress. A D-optimal split-plot design with 36 formulations each for anti-ILT4/anti-PD-1 co-formulated product, anti-ILT4 single entity (SE), and pembrolizumab SE for a total of 108 formulations was approved for this program. Since light stress is hard to change factor, the 36 formulations (data not shown) had 6 whole plots with light stress level constant for each of the whole plots. Each whole plot was further divided by 2 subplots to keep L-Met levels same within a whole plot. The studied ranges for the five factors are given in Table 7.

TABLE 7
Study to Evaluate Oxidation Sensitivity of anti-ILT4/anti-PD-1 co-formulation

| | | | | Fill | L- | | Time |
|---|---|---|---|---|---|---|---|
| Factor | Metal¹ | Peroxide² | Light³ | Volume⁴ | Methionine | Temperature | Points⁵ |
| Number of | 3 | 3 | 3 | 3 | 3 | 2 | 3 |
| Levels | | | | | | | |
| Level 1 | 0 ppb | 0 ppb | 0.0X | 1.2 mL | 7 mM | 5° C. | Initial |
| | | | ICH | | | | |
| Level 2 | 25 ppb | 20 ppb | 0.2X | 2.2 mL | 10 mM | 40° C. | 2 or 4 |
| | | | ICH | | | | weeks |
| Level 3 | 250 ppb | 200 ppb | 0.4X | 3.2 mL | 15 mM | N/A | 4 or 12 |
| | | | ICH | | | | weeks |

N/A = Not applicable
¹Metal will be tested by spiking equal amounts of FeCl₂ and CuCl₂. That is, 100 ppb Metal corresponds to a solution containing 50 ppb FeCl₂ and 50 ppb CuCl₂
²Peroxide will be tested by spiking equal amounts of t-BHP and AAPH. That is, 100 ppb Peroxide corresponds to a solution containing 50 ppb t-BHP and 50 ppb AAPH
³1X ICH Q1B exposure level corresponds to 1.2 million lux hours of white light and 200 watt hours/m² of UV energy
⁴Filling volume serves as surrogate to control O₂ level
⁵All samples will be staged on stability (5° C. for 0, 4, and 12 weeks; 40° C. for 0, 2 and 4 weeks)

### Example 4: Preparation of Metal and Peroxide Spiked Vials

A diluent (consisted of a protein-free solution) with a similar composition as anti-ILT4/anti-PD-1 co-formulation, anti-ILT4 SE and pembrolizumab SE stock solutions was used for the sample preparations. The metal stock solution included equal amounts of FeCl₂ and CuCl₂. Specifically, 100 ppb metal stock solution included to 50 ppb FeCl₂ and 50 ppb CuCl₂. The peroxide stock solution included equal parts t-BHP and AAPH. Specifically, 100 ppb peroxide stock solution included 50 ppb t-BHP and 50 ppb AAPH. Using the diluent, peroxide stock solution, and metal stock solution, appropriate amounts of these solutions were added to the filtered anti-ILT4/anti-PD-1 co-formulation, anti-ILT4 SE and pembrolizumab SE stock formulations in a proportion of 1 μL of diluent, peroxide stock solution, or metal stock solution for each 1 mL of stock formulation used. As such, multiple combinations of peroxide and metal concentrations were targeted. Specifically, evaluated peroxide concentrations were 0, 20 and 200 ppb, while those for metal were 0, 25 and 250 ppb. When considering the different L-Methionine concentrations (7, 10 and 15 mM), fill volumes (1.2, 2.2 and 3.2 mL) and levels of light exposure (0, 0.2× and 0.4×ICH CWL+UV), a total number of 108 independent formulations for anti-ILT4/anti-PD-1 co-formulation, anti-ILT4 SE and pembrolizumab SE were prepared. Table 7 summarizes the oxidation-stress conditions for each of the 108 formulations prepared, as well as the volumes of stock solutions used to prepare them. For the study, PS-80 levels were kept constant in all formulations and degradation was monitored over study parameters and duration.

Filtered anti-ILT4/anti-PD-1 co-formulation, anti-ILT4 SE and anti-ILT4 SE formulations were filled into 2R vials with variable fill volumes (1.2, 2.2 and 3.2 mL) in a laminar flow hood. The different fill volumes used in this study were selected to control the amount of oxygen available in the vials. A 2R vial was used instead of the intended commercial images (20 mL and 30 mL vials for Single Entity and Co-Formulation images, respectively) as a scale-down model and to minimize material usage.

The same 36 formulations (data not shown) were prepared for anti-ILT4/anti-PD-1 CF, anti-ILT4 SE, and pembrolizumab SE. The stock formulation corresponded to a formulation stocks of the corresponding coformulation or single entity, and depended on L-Methionine concentration (7, 10 or 15 mM).

### Example 5: Light Exposure and Stability Study

All vials from the same formulation set (i.e., anti-ILT4/anti-PD-1 co-formulation, anti-ILT4 SE or pembrolizumab SE) were placed in the photostability chamber at 25° C. for light exposure (0, 0.2× and 0.4×ICH CWL+UV). Samples intended for OX ICH light exposure were protected from light; nonetheless, these samples were also left inside chamber for the duration of the experiment. Samples intended for 0.2× and 0.4×ICH were placed inside the chamber in a horizontal position to maximize exposure.

After all vials were exposed to light, samples were staged on stability at 5° C. for up to 12 weeks and 40° C. for up to 4 weeks as per the protocol in Table 2. At each stability time point, samples were pulled and frozen at −80° C. prior to submission for analytical testing (with appropriate controls).

### Example 6: Qualitative Analysis

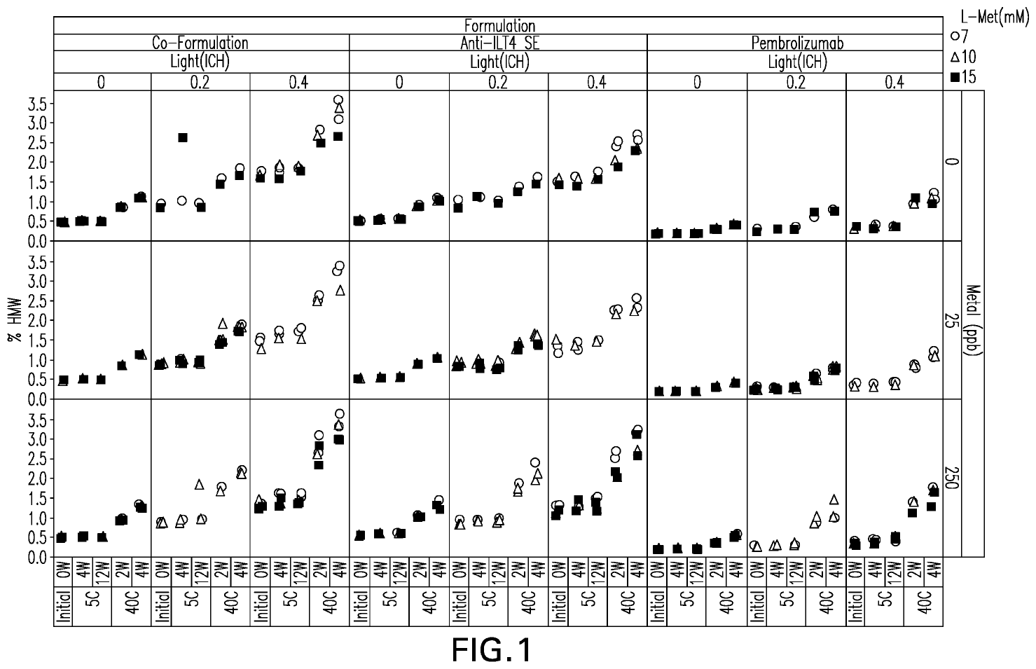

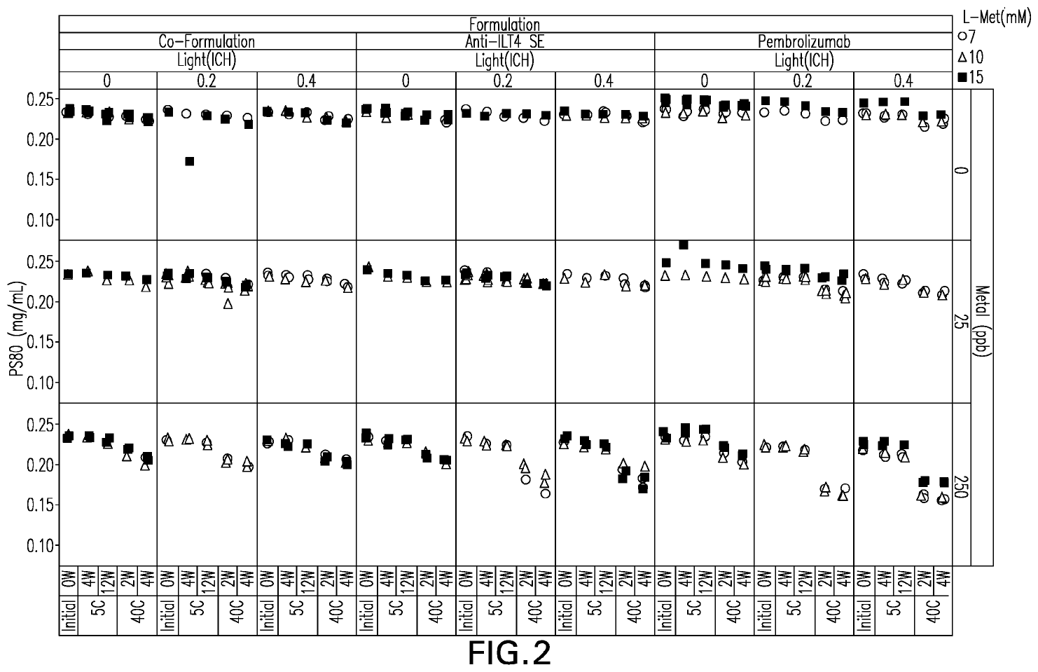

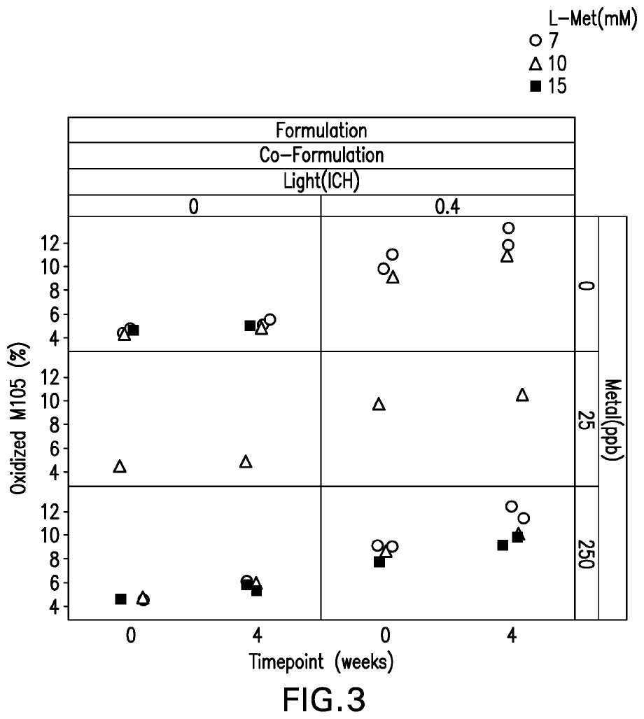

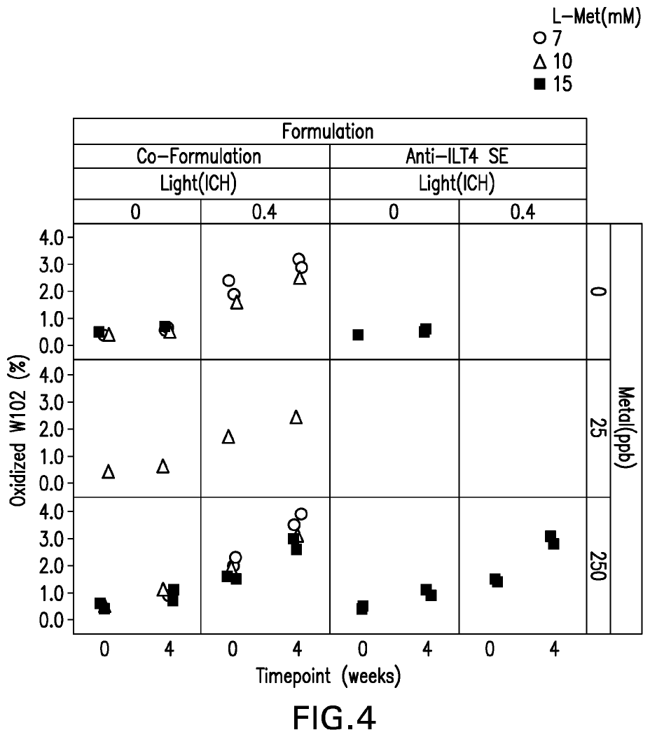

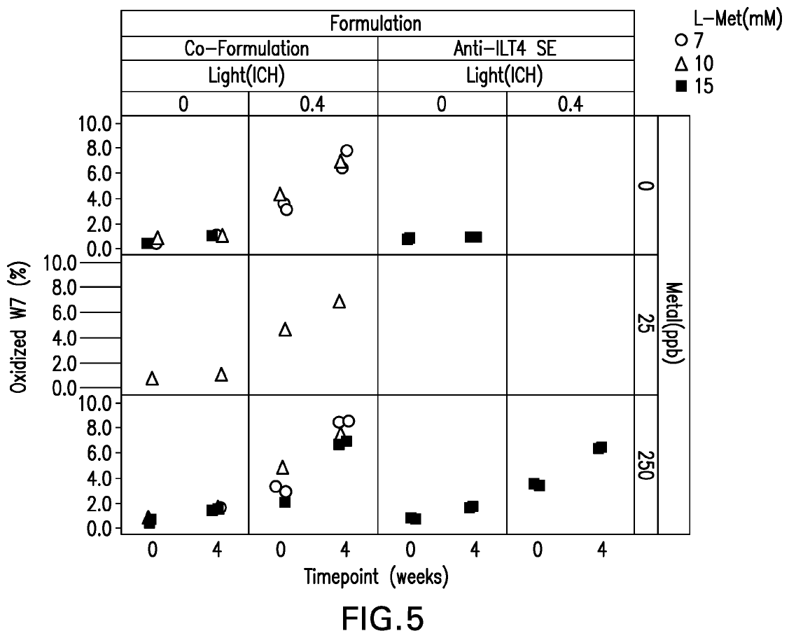

Visual analysis of analytical data suggested that there was no impact from increasing the residual peroxide levels (from 0-200 ppb) or from changes in fill volume (from 1.2 to 3.2 mL). On the other hand, changes in light exposure (from 0 to 0.4×ICH) and residual metal content (0-250 ppb) lead to increasing drug product degradation, as it was observed by change in high molecular weight species from UP-SEC (FIG. 1), degradation of PS80 (FIG. 2), and oxidation of Methionine and Tryptophan residues from Reduced peptide mapping (FIG. 3, FIG. 4, and FIG. 5) and HIC (FIG. 6). Note that the observed increase in physical and chemical degradation was detected for samples stored at 40° C. up to 4 weeks, while no change was observed on those samples stored at 5° C. for up to 3 months. Results from other attributes such as charged variants (by IEX) and subvisible particulate (by MFI) show minimal to no change of varying peroxide, metal, light of filling volume levels as captured in data summary.

Overall inspection of data from the study showed that exposure to light and metal stresses were the most significant factors affecting the oxidation of anti-ILT4/anti-PD-1 coformulation. Nonetheless, the effects from these stressors were reduced by increasing L-Methionine concentration. As such, the data suggested that a concentration of 15 mM of L-Methionine provides the lowest levels of aggregation and chemical degradation. Notably, such L-Methionine concentration was only effective for anti-ILT4/anti-PD-1 coformulation, as no improvement in physical and chemical attributes for anti-ILT4 monoclonal antibody SE and pembrolizumab SE are observed at L-Methionine concentrations larger than 10 mM.

### Example 7: UP-SEC (Ultra-Performance Size Exclusion Chromatography)

Increases in high molecular weight species (HMW) and low molecular weight species (LMW) indicate protein degradation via aggregation and fragmentation. The plots (data not shown) indicated that higher light stress levels provide higher % HMW and % LMW and increases with time while it gives lower monomer and further decreases with time for the samples at 40 C.

Statistical analysis of the data was performed to evaluate the effects from the different formulation and process factors (i.e., L-methionine concentration, peroxide content, metal content, light exposure, and filling volume). Based on this analysis, contour plots were given for % HMW only for all the three formulations.

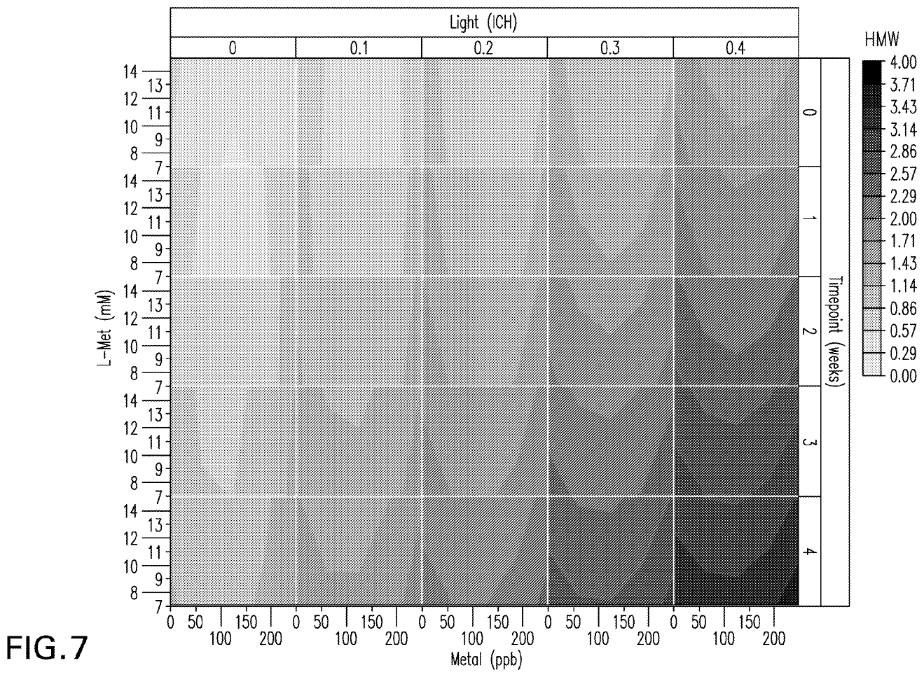

FIG. 7 depicts contour plots for the HMW coformulation with the predicted values at 40° C. condition from the model fits over different settings of the three critical parameters (L-Methionine, light exposure, metal content) over time. The contour plots show that as light stress increases and with lower levels of L-Met the predicted levels of HMW increases.

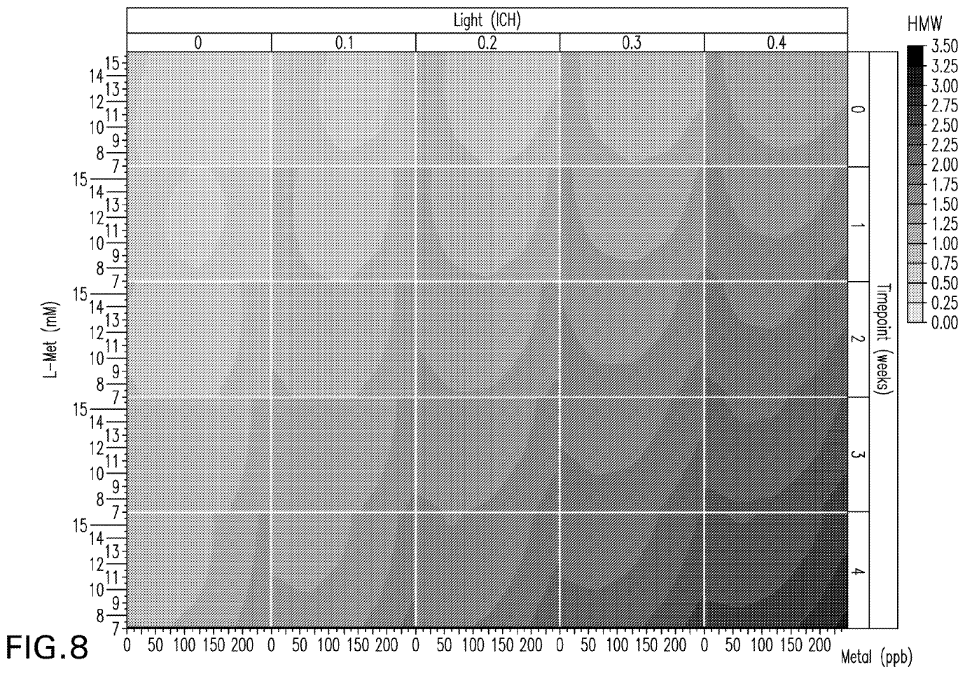

Similar to the co-formulated formulation, the contour plot for the anti-ILT4 monoclonal antibody SE, as shown in FIG. 8, shows that as light exposure and Metal content increases, the levels of HMW also increased when L-Methionine concentration was below 10 mM.

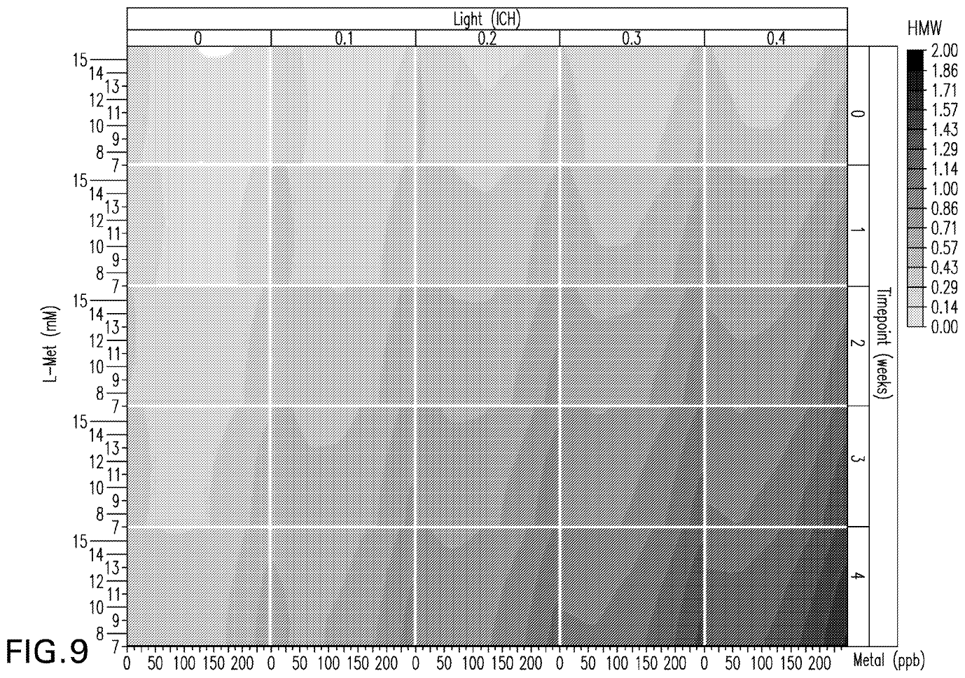

The contour plots for pembrolizumab SE are shown in FIG. 9. Similarly to anti-ILT4 SE, statistical analysis shows that as light exposure and Metal content increases, the levels of HMW also increased when L-Methionine concentration was below 10 mM.

Change in charge variants could be results of oxidation, deamidation and isomerization. The variability plots (data not shown) indicated that higher light stress levels increased % Acidic Variants and % Basic Variants and decreased % Main at all conditions. The same conclusions were seen in the charge variants of pembrolizumab (on both the co-formulation and pembrolizumab SE).

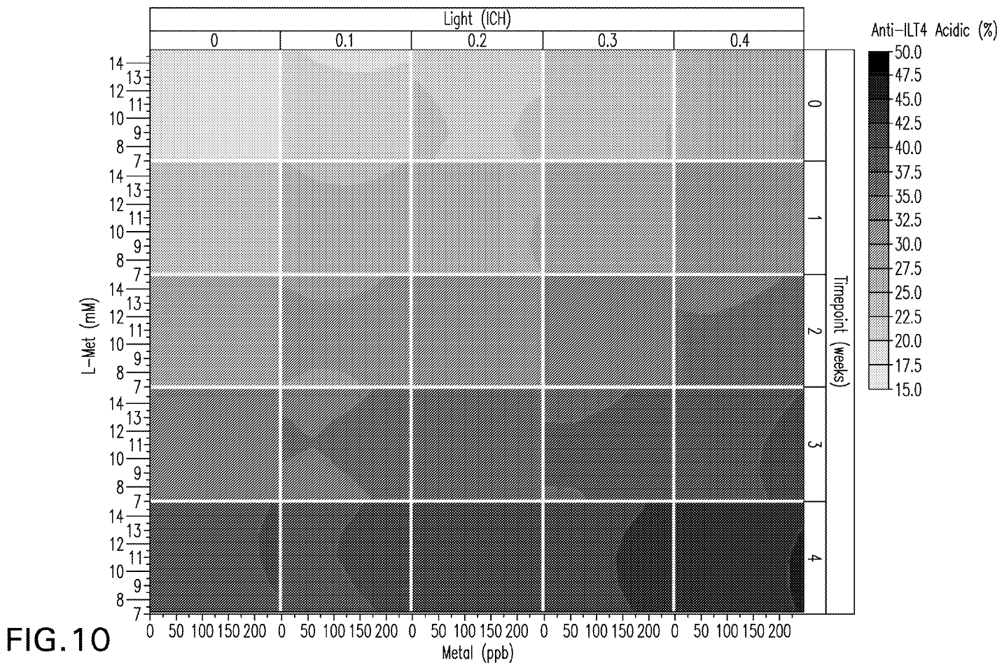

Statistical analysis of the data was performed to evaluate the effects from the different formulation and process factors (i.e., L-methionine concentration, peroxide content, metal content, light exposure, and filling volume). Based on this analysis, contour plots are given for % Acidic Variants, % Main and % Basic Variants for all the three formulations in FIGS. 10-18.

The contour plot of FIG. 10 shows predicted values of % Acidic Variants for anti-ILT4 in the co-formulation at different factor levels at 40° C. for 4 weeks.

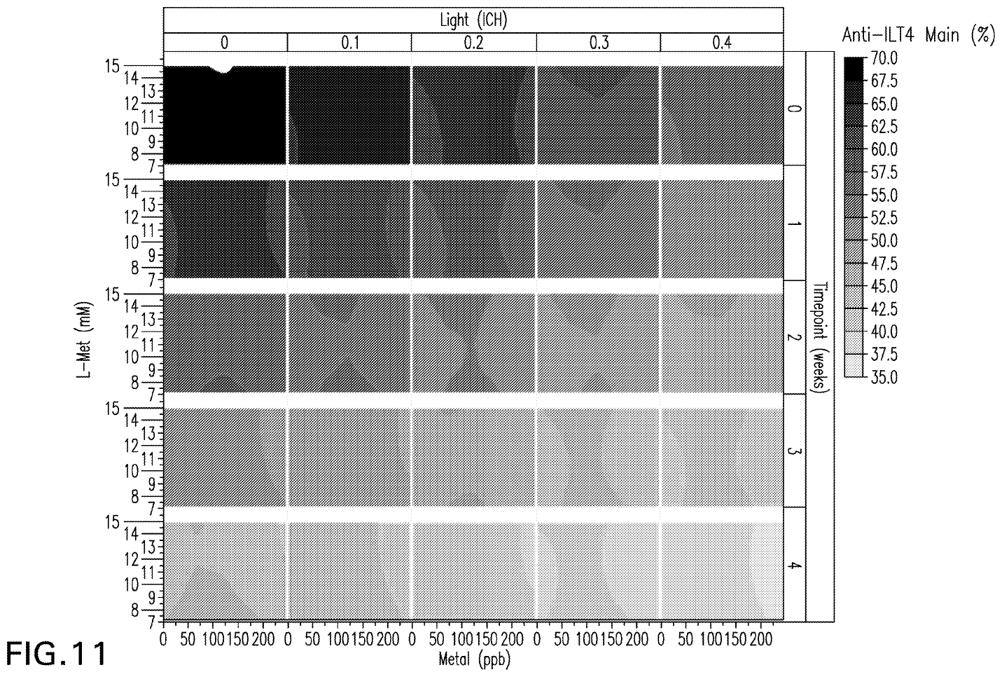

The contour plot of FIG. 11 shows the % Main for anti-ILT4 in the co-formulation as a function of different factors (L-Methionine concentration, Light exposure and Metal content). The figure illustrates that increase in Light stress decreases % Main for anti-ILT4.

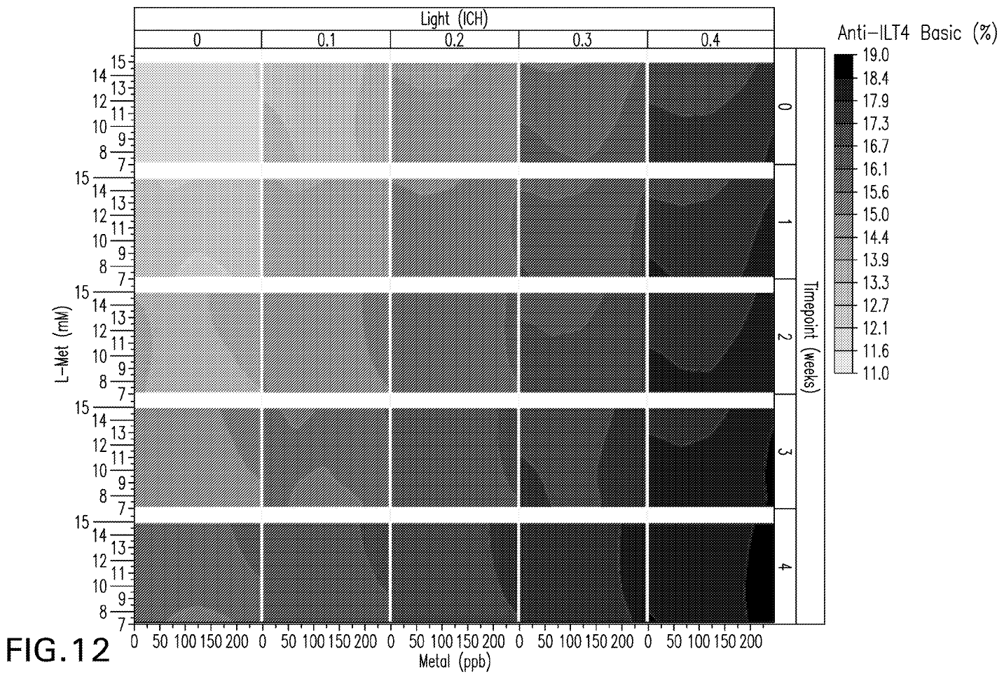

The contour plot of FIG. 12 shows of the % Basic Variants for anti-ILT4 in the co-formulation as a function of different factors (L-Methionine concentration, Light exposure and Metal content). The figure illustrates that increase in Light stress increases % Basic Variants for anti-ILT4.

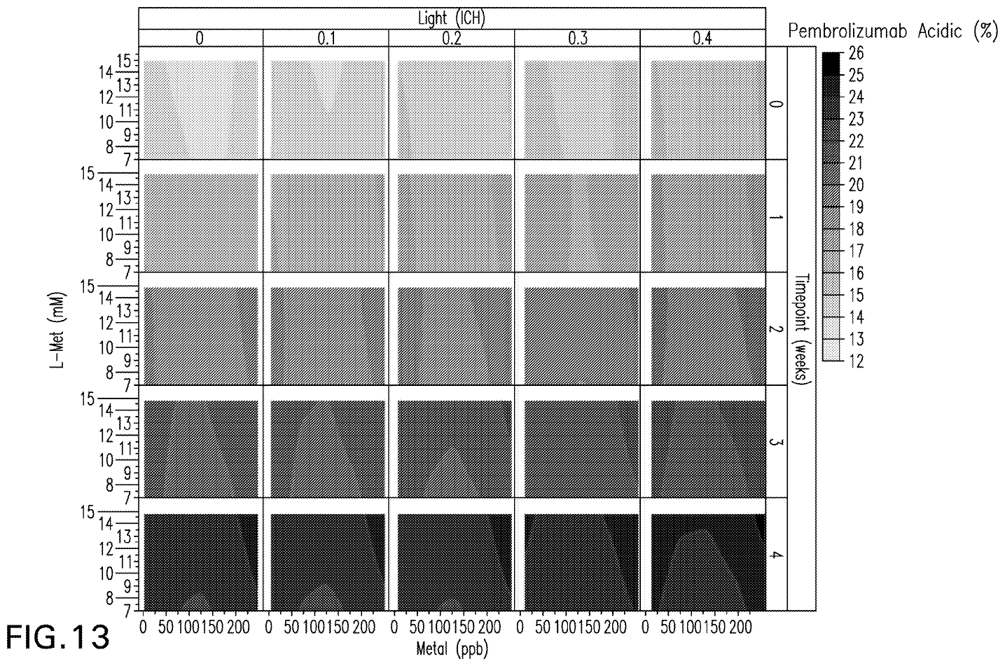

The contour plot of FIG. 13 shows the predicted values from the model fits over different settings of the three parameters (L-Methionine concentration, Light exposure, Metal content) over time. The contour plots show that as Light Stress increases, the predicted levels of % Acidic Variants for pembrolizumab in the co-formulation remain constant.

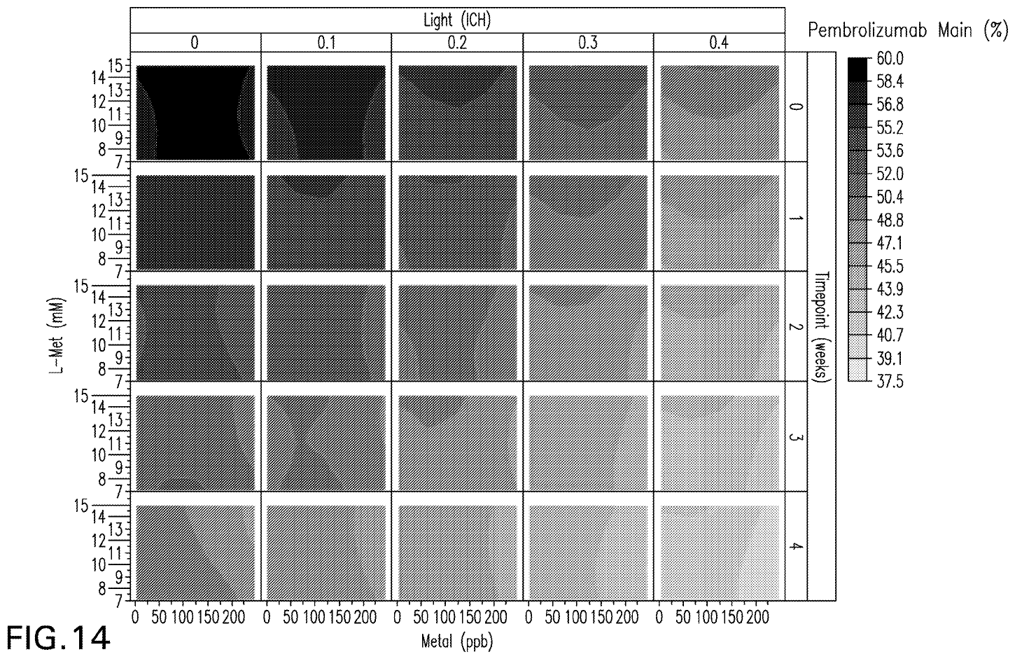

The contour plot of FIG. 14 shows the % Main for pembrolizumab in the co-formulation as a function of different factors (L-Methionine concentration, Light exposure, and Metal content). The figure illustrates that increase in Light stress and Metal content decrease % Main for pembrolizumab. By contrast, increasing L-Methionine concentration above 10 mM reduces the rate of Main decrease upon light stress.

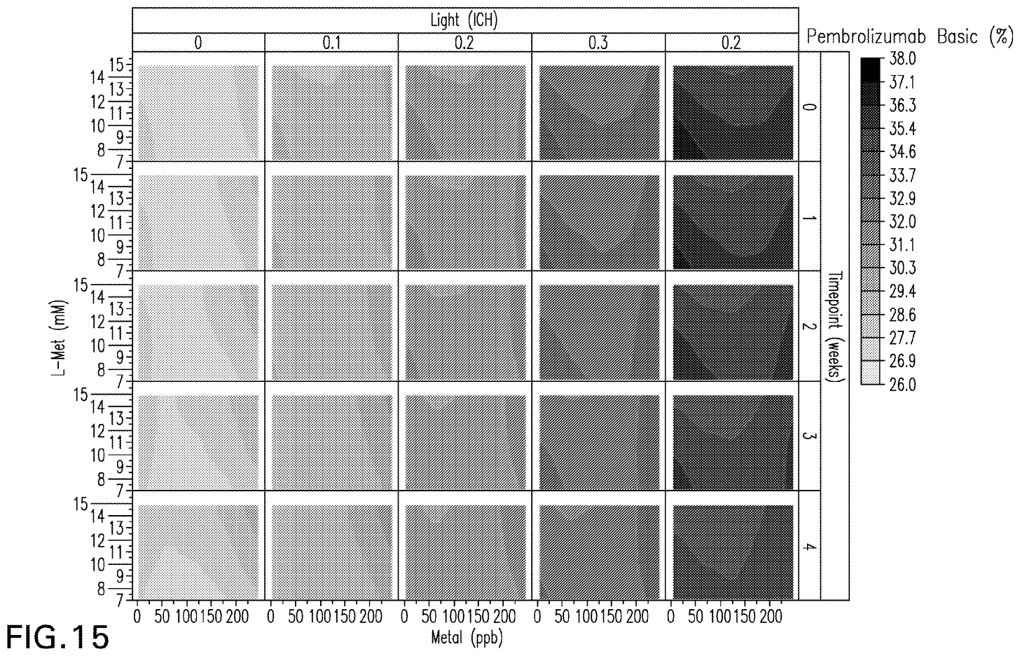

The contour plot of FIG. 15 shows the % Basic Variants for pembrolizumab in the co-formulation as a function of different factors (L-Methionine concentration, Light exposure, and Metal content). The figure illustrates that increase in Light stress decreases % Basic Variants for pembrolizumab, while increasing L-Methionine concentration above 10 mM reduces the rate of Main decrease upon light stress.

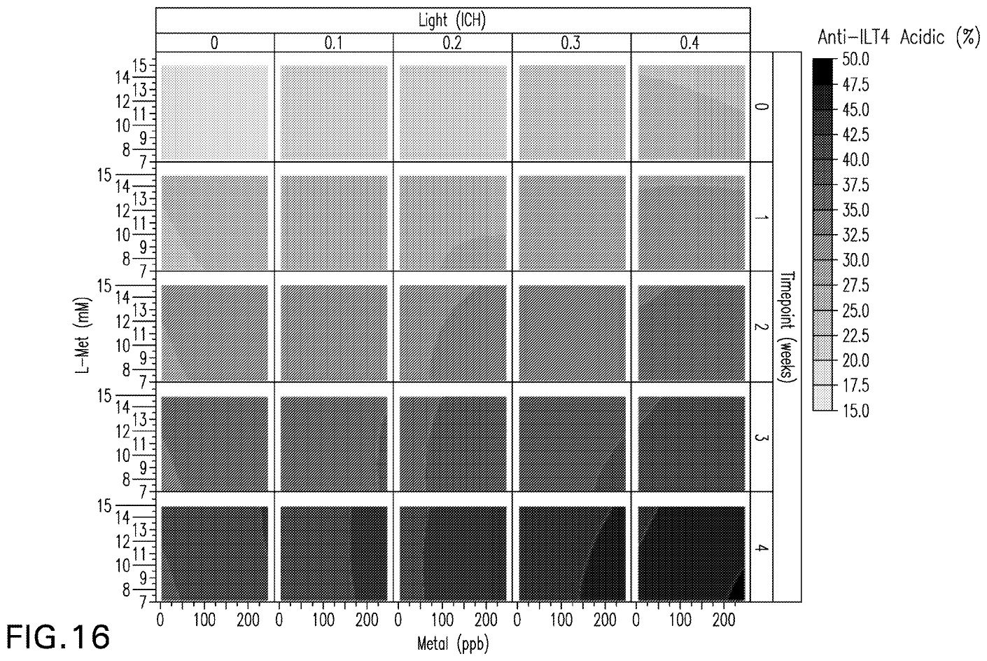

The contour plot of FIG. 16 shows the predicted values from the model fits over different settings of the three parameters (L-Methionine concentration, Light exposure, Metal content) over time. The contour plots show that as Light Stress increases, the predicted levels of % Acidic Variants for anti-ILT4 in the anti-ILT4 SE formulation increases.

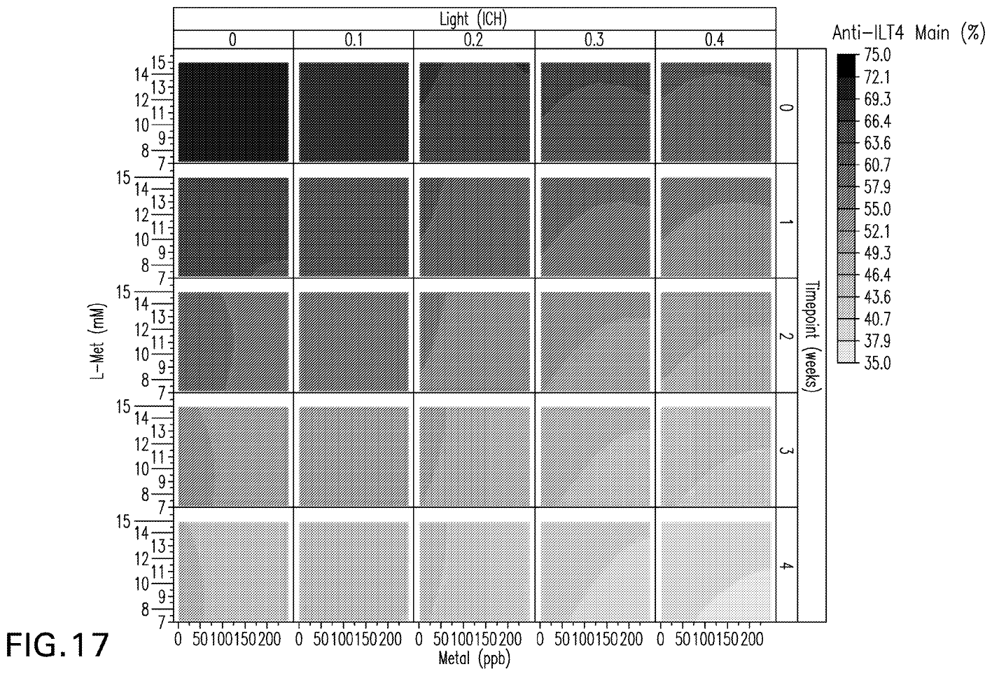

The contour plot of FIG. 17 shows the % Main for anti-ILT4 in the anti-ILT4 SE formulation as a function of different factors (L-Methionine concentration, Light exposure and Metal content). The figure illustrates that increase in Light stress and Metal content decrease % Main for anti-ILT4.

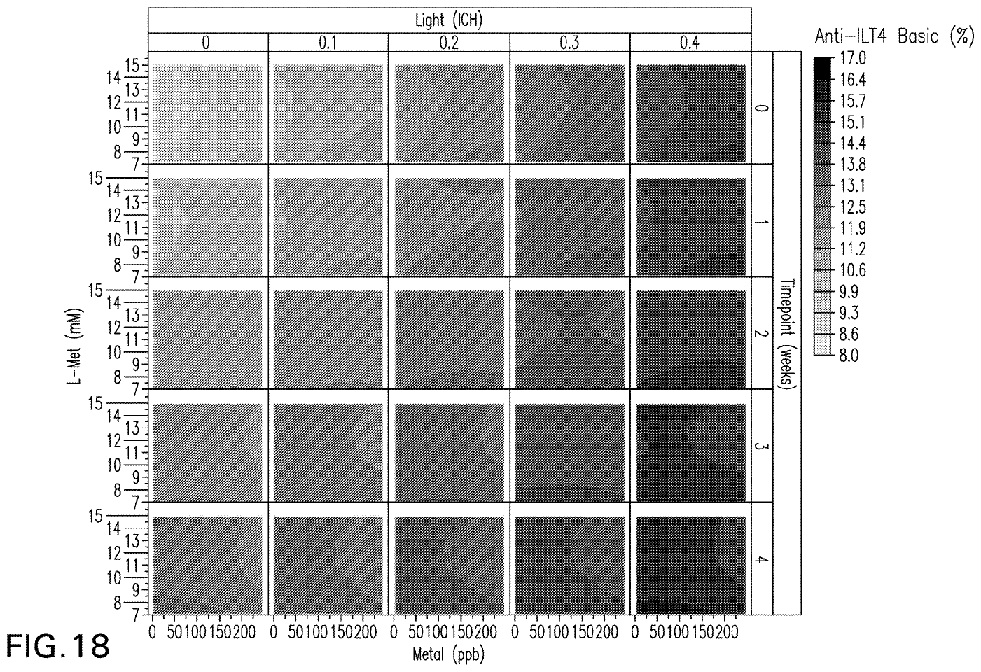

The contour plot of FIG. 18 shows the % Basic Variants for anti-ILT4 in the anti-ILT4 SE formulation as a function of different factors (L-Methionine concentration, Light exposure and Metal content). The figure illustrates that increase in Light stress increases % Basic Variants for anti-ILT4.

Overall, the data provided key information on the five critical parameters used to study the oxidation process. In some of the responses, some out of trend values introduced noise in the data and making it hard for statistical interpretations. But a holistic review of the data based on statistical models, prediction profilers and contour plots were done to propose a final formulation for the co-formulation and anti-ILT4 SE formulation. Based on the responses of attributes measuring oxidation, high levels of Light Stress consistently had a significant effect on all responses. L-Methionine concentration, and Peroxide and Metal content were also significant for some of the responses. Metal content had significant effect in some cases for pembrolizumab SE formulation. Filling weight did not have any significant effect in most of the responses, and the factor range can be kept at the proposed levels of 1.2 mL to 3.2 mL. For prediction purposes, the filling volume was set at the target level of 2.2 mL.

Based on statistical predictions of the key responses (UPSEC, IEX, PS-80, L-Methionine, Turbidity), data summaries, graphs and scientific input, the final formulations chosen for SE and co-formulated product are given in Table 8.

TABLE 8
Final formulation composition of Co-formulation
and anti-ILT4 SE formulation

| | L-Met | Metal | Peroxide | Light |
|---|---|---|---|---|
| | (mM) | (ppb) | (ppb) | (ICH) |
| Co-formulation | 15 | 50 | 50 | 0.2X |
| Anti-ILT4 SE formulation | 10 | 50 | 50 | 0.2X |

Long-term stability for the two lead formulation prototypes was evaluated in this study. The final two compositions for the anti-ILT4/anti-PD-1 coformulations (with 10 mM and 15 mM L-Methionine, respectively) were staged on stability for 5° C. at 12 months and at 25° C. and 40° C. for 6 months. The formulation composition for the Co-formulation 1 (CF 1, 15 mM L-Methionine) and Co-formulation 2 (CF 2, 10 mM L-Methionine) are captured in Table 9. All the data is captured in Tables 10-12. Excess 5 mM of L-Methionine was spiked as powder and dissolved by gentle mixing. Samples were analyzed by HIC, IEX, SEC, L-Methionine content, PS-80 content, Sub-Visible Particles and Reduced Peptide Mapping assays.

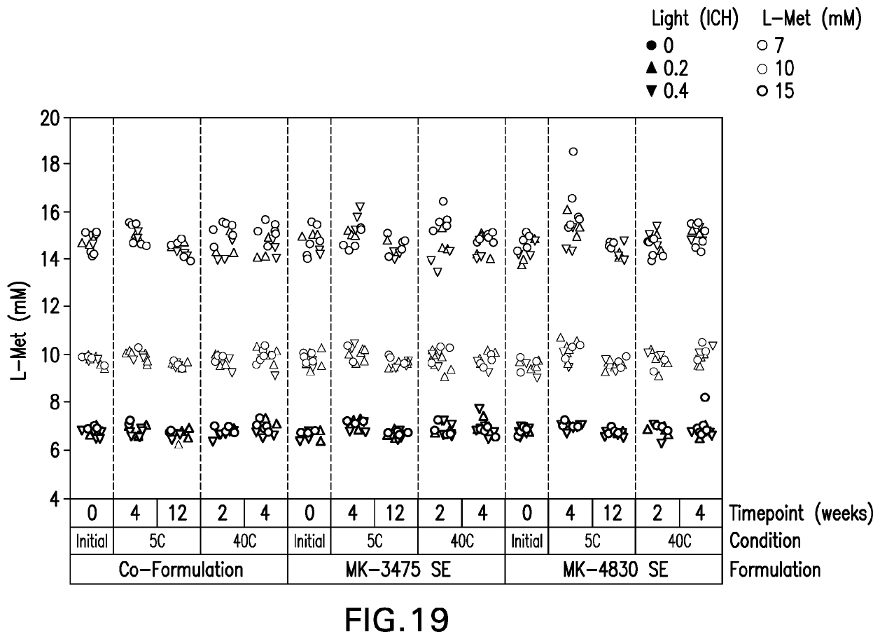

As shown in FIG. 19, the L-Methionine concentration at all stability time points for all the three formulations. The results show negligible effects from all the different factors on the stability of L-Methionine.

TABLE 9
Composition of anti-ILT4/anti-PD-1 coformulations
for Pre-probe stability

| | | Concentration (mg/mL) | |
|---|---|---|---|
| | | Co-formulation | Co-formulation |
| Component | Function | 1 (CF 1) | 2 (CF 2) |
| anti-ILT4 | Active | 32.0 | 33.34 |
| monoclonal | pharmaceutical | | |
| antibody | ingredient | | |
| pembrolizumab | Active | 8.0 | 8.33 |
| | pharmaceutical | | |
| | ingredient | | |
| L-Histidine | Buffer | 0.56 | 0.56 |
| L-Histidine | Buffer | 1.35 | 1.35 |
| Monohydrochloride | | | |
| Monohydrate | | | |
| L-Methionine | Stabilizer | 2.24 (15 mM) | 1.49 (10 mM) |
| Sucrose | Stabilizer/ | 70.0 | 70.0 |
| | Tonicity | | |
| | Modifier | | |
| Polysorbate 80 | Surfactant | 0.25 | 0.25 |
| Water for Injection | Solvent | q.s. to 1.0 mL | q.s. to 1.0 mL |

TABLE 10
Summary of stability data for anti-ILT4/anti-PD-1 coformulation prototypes CF 1 and CF 2 at 5° C.

| | Time Point (months) | | | | | | | | | |
|---|---|---|---|---|---|---|---|---|---|---|
| | Initial | | 1 | | 3 | | 6 | | 12 | |
| Analysis | CF1 | CF2 | CF1 | CF2 | CF1 | CF2 | CF1 | CF2 | CF1 | CF2 |

UP-SEC

| | | | | | | | | | | |
|---|---|---|---|---|---|---|---|---|---|---|
| HMW (%) | 0.44 | 0.43 | 0.50 | 0.50 | 0.48 | 0.48 | 0.63 | 0.63 | 0.69 | 0.72 |
| Monomer | 99.6 | 99.6 | 99.5 | 99.5 | 99.5 | 99.5 | 99.4 | 99.4 | 99.3 | 99.3 |
| (%) | | | | | | | | | | |
| LMW (%) | 0.00 | 0.00 | 0.00 | 0.00 | 0.00 | 0.00 | 0.01 | 0.01 | 0.00 | 0.00 |

HP-IEX

| | | | | | | | | | | |
|---|---|---|---|---|---|---|---|---|---|---|
| Total Acid | 13.1 | 13.3 | 13.6 | 13.6 | 15.6 | 15.9 | 12.8 | 13.1 | 13.7 | 14.3 |
| anti-PD-1 | | | | | | | | | | |
| Total Main | 64.4 | 64.3 | 63.5 | 64.6 | 56.7 | 56.4 | 62.9 | 62.8 | 60.6 | 59.8 |
| anti-PD-1 | | | | | | | | | | |
| Total Basic | 22.5 | 22.4 | 22.9 | 21.8 | 27.7 | 27.7 | 24.3 | 24.1 | 25.7 | 26.0 |
| anti-PD-1 | | | | | | | | | | |
| Total Acid | 19.3 | 19.2 | 20.1 | 20.3 | 19.2 | 18.4 | 17.9 | 18.0 | 20.2 | 20.2 |
| anti-ILT4 | | | | | | | | | | |
| Total Main | 69.7 | 69.6 | 69.3 | 69.1 | 66.5 | 67.2 | 70.3 | 70.2 | 67.9 | 67.9 |
| anti-ILT4 | | | | | | | | | | |
| Total Basic | 11.0 | 11.2 | 10.6 | 10.6 | 14.3 | 14.4 | 11.8 | 11.8 | 11.9 | 11.9 |
| anti-ILT4 | | | | | | | | | | |

L-Met

| | | | | | | | | | | |
|---|---|---|---|---|---|---|---|---|---|---|
| Amount L- | 15.2 | 10.5 | 14.6 | 10.0 | 13.7 | 9.3 | 15.0 | 9.7 | 13.9 | 9.8 |
| Met (mM) | | | | | | | | | | |

PS-80

| | | | | | | | | | | |
|---|---|---|---|---|---|---|---|---|---|---|
| Amount | 0.23 | 0.23 | 0.24 | 0.24 | 0.22 | 0.23 | 0.21 | 0.21 | 0.22 | 0.21 |
| PS-80 | | | | | | | | | | |
| (mg/mL) | | | | | | | | | | |

MFI

| | | | | | | | | | | |
|---|---|---|---|---|---|---|---|---|---|---|
| >=1 | 324 | 380 | 345 | 662 | 3170 | 2826 | 7259 | 4878 | 9372 | 9241 |
| um:Conc | | | | | | | | | | |
| >=2 | 78 | 76 | 65 | 136 | 343 | 742 | 920 | 666 | 895 | 931 |
| um:Conc | | | | | | | | | | |
| >=5 | 14 | 21 | 5 | 21 | 44 | 88 | 42 | 35 | 46 | 46 |
| um:Conc | | | | | | | | | | |
| >=10 | 3 | 5 | 5 | 0 | 7 | 0 | 0 | 3 | 3 | 10 |
| um:Conc | | | | | | | | | | |
| >=25 | 0 | 0 | 0 | 0 | 0 | 0 | 0 | 0 | 0 | 3 |
| um:Conc | | | | | | | | | | |
| >=50 | 0 | 0 | 0 | 0 | 0 | 0 | 0 | 0 | 0 | 0 |
| um:Conc | | | | | | | | | | |

Red-PM

| | | | | | | | | | | |
|---|---|---|---|---|---|---|---|---|---|---|
| M253 | 1.3 | 1.4 | NT | NT | 1.7 | 1.7 | 1.5 | 1.4 | 1.4 | 1.5 |
| (Share) | | | | | | | | | | |
| M429 | 0.7 | 0.7 | NT | NT | 0.8 | 0.8 | 1.0 | 0.9 | 1.2 | 1.1 |
| (Share) | | | | | | | | | | |
| W7 (anti- | 0.8 | 0.8 | NT | NT | 0.8 | 0.9 | 0.8 | 0.9 | 0.5 | 0.5 |
| ILT4) | | | | | | | | | | |
| W102 | 0.3 | 0.5 | NT | NT | 0.6 | 0.6 | 0.4 | 0.4 | 0.3 | 0.3 |
| (anti-ILT4) | | | | | | | | | | |
| M105 | 3.6 | 4.0 | NT | NT | 4.9 | 4.9 | 5.2 | 4.9 | 4.7 | 4.8 |
| (anti-PD-1) | | | | | | | | | | |

TABLE 11
Summary of stability data for anti-ILT4/anti-PD-1 coformulation prototypes CF 1 and CF 2 at 25° C.

| | Time Point (months) | | | | | | | | | |
|---|---|---|---|---|---|---|---|---|---|---|
| | Initial | | 1 | | 2 | | 3 | | 6 | |
| Analysis | CF1 | CF2 | CF1 | CF2 | CF1 | CF2 | CF1 | CF2 | CF1 | CF2 |

UP-SEC

| | | | | | | | | | | |
|---|---|---|---|---|---|---|---|---|---|---|
| HMW (%) | 0.44 | 0.43 | 0.70 | 0.72 | 0.72 | 0.76 | 0.82 | 0.86 | 1.18 | 1.24 |
| Monomer | 99.6 | 99.6 | 99.3 | 99.3 | 99.3 | 99.2 | 99.2 | 99.1 | 98.8 | 98.7 |
| (%) | | | | | | | | | | |
| LMW (%) | 0.00 | 0.00 | 0.00 | 0.01 | 0.01 | 0.01 | 0.01 | 0.01 | 0.03 | 0.03 |

HP-IEX

| | | | | | | | | | | |
|---|---|---|---|---|---|---|---|---|---|---|
| Total Acid | 13.1 | 13.3 | 15.5 | 15.2 | 17.0 | 16.9 | 23.8 | 23.8 | 21.3 | 21.2 |
| anti-PD-1 | | | | | | | | | | |
| Total Main | 64.4 | 64.3 | 61.2 | 61.6 | 59.2 | 58.8 | 50.3 | 50.7 | 56.3 | 55.6 |
| anti-PD-1 | | | | | | | | | | |
| Total Basic | 22.5 | 22.4 | 23.2 | 23.2 | 23.8 | 24.3 | 25.9 | 25.6 | 22.3 | 23.2 |
| anti-PD-1 | | | | | | | | | | |
| Total Acid | 19.3 | 19.2 | 22.0 | 22.2 | 25.1 | 24.9 | 26.6 | 26.5 | 34.2 | 33.8 |
| anti-ILT4 | | | | | | | | | | |
| Total Main | 69.7 | 69.6 | 65.5 | 65.6 | 60.9 | 61.0 | 57.9 | 58.0 | 51.5 | 51.7 |
| anti-ILT4 | | | | | | | | | | |
| Total Basic | 11.0 | 11.2 | 12.5 | 12.2 | 14.0 | 14.1 | 15.5 | 15.5 | 14.3 | 14.5 |
| anti-ILT4 | | | | | | | | | | |

L-Met

| | | | | | | | | | | |
|---|---|---|---|---|---|---|---|---|---|---|
| Amount L- | 15.2 | 10.5 | 14.5 | 9.9 | 14.8 | 9.6 | 14.0 | 9.3 | 14.4 | 9.6 |
| Met (mM) | | | | | | | | | | |

PS-80

| | | | | | | | | | | |
|---|---|---|---|---|---|---|---|---|---|---|
| Amount PS- | 0.23 | 0.23 | 0.23 | 0.23 | 0.22 | 0.23 | 0.22 | 0.22 | 0.19 | 0.19 |
| 80 (mg/mL) | | | | | | | | | | |

MFI

| | | | | | | | | | | |
|---|---|---|---|---|---|---|---|---|---|---|
| >=1 | 324 | 380 | 815 | 666 | 1804 | 1237 | 1026 | 955 | 2822 | 2622 |
| um:Conc | | | | | | | | | | |
| >=2 | 78 | 76 | 212 | 189 | 363 | 377 | 287 | 271 | 639 | 559 |
| um:Conc | | | | | | | | | | |
| >=5 | 14 | 21 | 12 | 14 | 17 | 17 | 51 | 69 | 56 | 95 |
| um:Conc | | | | | | | | | | |
| >=10 | 3 | 5 | 3 | 0 | 5 | 0 | 12 | 35 | 10 | 23 |
| um:Conc | | | | | | | | | | |
| >=25 | 0 | 0 | 3 | 0 | 0 | 0 | 0 | 0 | 3 | 5 |
| um:Conc | | | | | | | | | | |
| >=50 | 0 | 0 | 0 | 0 | 0 | 0 | 0 | 0 | 0 | 0 |
| um:Conc | | | | | | | | | | |

Red-PM

| | | | | | | | | | | |
|---|---|---|---|---|---|---|---|---|---|---|
| M253 | 1.3 | 1.4 | NT | NT | 1.3 | 1.4 | 1.8 | 1.8 | 1.6 | 1.6 |
| (Share) | | | | | | | | | | |
| M429 | 0.7 | 0.7 | NT | NT | 0.7 | 0.7 | 0.8 | 0.8 | 1.1 | 1.0 |
| (Share) | | | | | | | | | | |
| W7 (anti- | 0.8 | 0.8 | NT | NT | 0.9 | 1.0 | 1.1 | 1.1 | 1.3 | 1.2 |
| ILT4) | | | | | | | | | | |
| W102 anti- | 0.3 | 0.5 | NT | NT | 0.6 | 0.6 | 0.7 | 0.7 | 0.6 | 0.6 |
| ILT4) | | | | | | | | | | |
| M105 (anti- | 3.6 | 4.0 | NT | NT | 3.9 | 4.4 | 5.1 | 5.0 | 5.5 | 5.8 |
| PD-1) | | | | | | | | | | |

TABLE 12
Summary of stability data for anti-ILT4/anti-PD-1 coformulation prototypes CF 1 and CF 2 at 40° C.

| | Time Point (months) | | | | | | | | | |
|---|---|---|---|---|---|---|---|---|---|---|
| | Initial | | 1 | | 2 | | 3 | | 6 | |
| Analysis | CF1 | CF2 | CF1 | CF2 | CF1 | CF2 | CF1 | CF2 | CF1 | CF2 |

UP-SEC

| | | | | | | | | | | |
|---|---|---|---|---|---|---|---|---|---|---|
| HMW (%) | 0.44 | 0.43 | 1.19 | 1.25 | 1.71 | 1.81 | 2.40 | 2.58 | 7.22 | 7.23 |
| Monomer | 99.6 | 99.6 | 98.8 | 98.7 | 98.2 | 98.1 | 97.5 | 97.3 | 92.2 | 92.2 |
| (%) | | | | | | | | | | |
| LMW (%) | 0.00 | 0.00 | 0.04 | 0.03 | 0.07 | 0.07 | 0.10 | 0.09 | 0.60 | 0.55 |

HP-IEX

| | | | | | | | | | | |
|---|---|---|---|---|---|---|---|---|---|---|
| Total Acid | 13.1 | 13.3 | 23.3 | 23.6 | 31.5 | 31.8 | 37.8 | 37.7 | NT | NT |
| anti-PD-1 | | | | | | | | | | |
| Total Main | 64.4 | 64.3 | 52.5 | 53.2 | 43.5 | 43.9 | 36.3 | 36.1 | NT | NT |
| anti-PD-1 | | | | | | | | | | |
| Total Basic | 22.5 | 22.4 | 24.2 | 23.2 | 25.0 | 24.3 | 25.8 | 26.2 | NT | NT |
| anti-PD-1 | | | | | | | | | | |
| Total Acid | 19.3 | 19.2 | 39.9 | 39.9 | 54.8 | 54.8 | 61.9 | 61.9 | NT | NT |
| anti-ILT4 | | | | | | | | | | |
| Total Main | 69.7 | 69.6 | 45.6 | 45.8 | 29.9 | 30.0 | 23.5 | 23.5 | NT | NT |
| anti-ILT4 | | | | | | | | | | |
| Total Basic | 11.0 | 11.2 | 14.5 | 14.4 | 15.3 | 15.3 | 14.6 | 14.6 | NT | NT |
| anti-ILT4 | | | | | | | | | | |

L-Met

| | | | | | | | | | | |
|---|---|---|---|---|---|---|---|---|---|---|
| Amount L- | 15.2 | 10.5 | 14.6 | 10.0 | 14.7 | 9.7 | 14.3 | 9.6 | 14.6 | 9.5 |
| Met (mM) | | | | | | | | | | |

PS-80

| | | | | | | | | | | |
|---|---|---|---|---|---|---|---|---|---|---|
| Amount PS- | 0.23 | 0.23 | 0.22 | 0.23 | 0.22 | 0.22 | 0.21 | 0.20 | 0.17 | 0.18 |
| 80 (mg/mL) | | | | | | | | | | |

MFI

| | | | | | | | | | | |
|---|---|---|---|---|---|---|---|---|---|---|
| >=1 | 324 | 380 | 1936 | 4929 | 594 | 1436 | 7932 | 1201 | 2424 | 2735 |
| um:Conc | | | | | | | | | | |
| >=2 | 78 | 76 | 483 | 1431 | 138 | 354 | 2081 | 363 | 441 | 606 |
| um:Conc | | | | | | | | | | |
| >=5 | 14 | 21 | 28 | 345 | 5 | 53 | 214 | 65 | 56 | 62 |
| um:Conc | | | | | | | | | | |
| >=10 | 3 | 5 | 5 | 104 | 0 | 3 | 21 | 14 | 3 | 7 |
| um:Conc | | | | | | | | | | |
| >=25 | 0 | 0 | 0 | 12 | 0 | 3 | 3 | 0 | 0 | 0 |
| um:Conc | | | | | | | | | | |
| >=50 | 0 | 0 | 0 | 3 | 0 | 0 | 0 | 0 | 0 | 0 |
| um:Conc | | | | | | | | | | |

Red-PM

| | | | | | | | | | | |
|---|---|---|---|---|---|---|---|---|---|---|
| M253 | 1.3 | 1.4 | NT | NT | 1.5 | 1.6 | 1.8 | 2.1 | 2.0 | 2.1 |
| (Share) | | | | | | | | | | |
| M429 | 0.7 | 0.7 | NT | NT | 0.8 | 0.9 | 1.0 | 1.1 | 2.1 | 1.1 |
| (Share) | | | | | | | | | | |
| W7 (anti- | 0.8 | 0.8 | NT | NT | 1.4 | 1.5 | 1.8 | 2.1 | 3.9 | 3.8 |
| ILT4) | | | | | | | | | | |
| W102 anti- | 0.3 | 0.5 | NT | NT | 0.8 | 0.8 | 1.0 | 1.1 | 1.5 | 1.6 |
| ILT4) | | | | | | | | | | |
| M105 (anti- | 3.6 | 4.0 | NT | NT | 4.3 | 5.0 | 5.7 | 6.9 | 8.3 | 8.7 |
| PD-1) | | | | | | | | | | |

### Example 8: Pre-Probe Stability Study for Anti-ILT4/Anti-PD-1 Coformulation

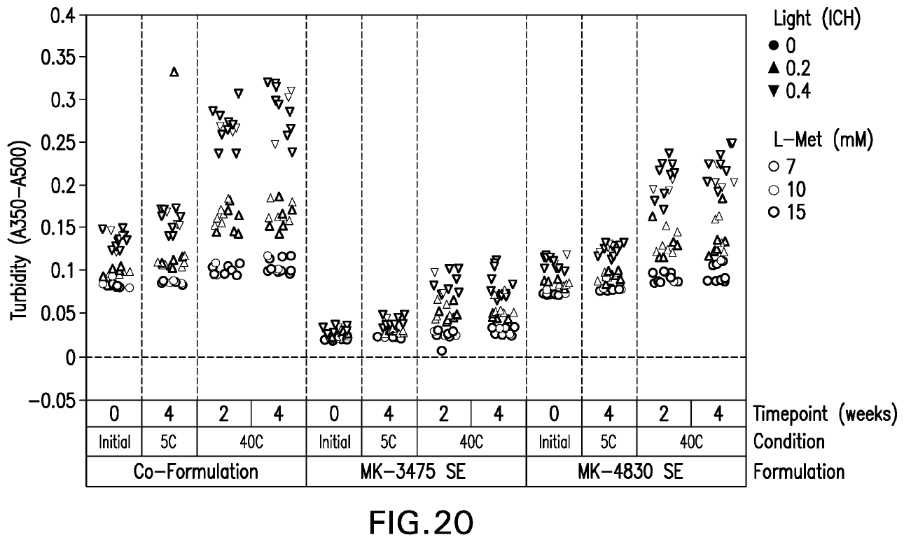

As shown in FIG. 20, the plot of turbidity values for all the three formulations at 5° C. and 40° C. conditions. Statistical analysis of the data in FIG. 20 was performed to evaluate the effects from the different formulation and process factors (i.e., L-methionine concentration, peroxide content, metal content, light exposure, and filling volume). Based on this analysis, prediction profilers are given for turbidity for all the three formulations. An increase in solution turbidity indicates increase in aggregation in protein formulations.

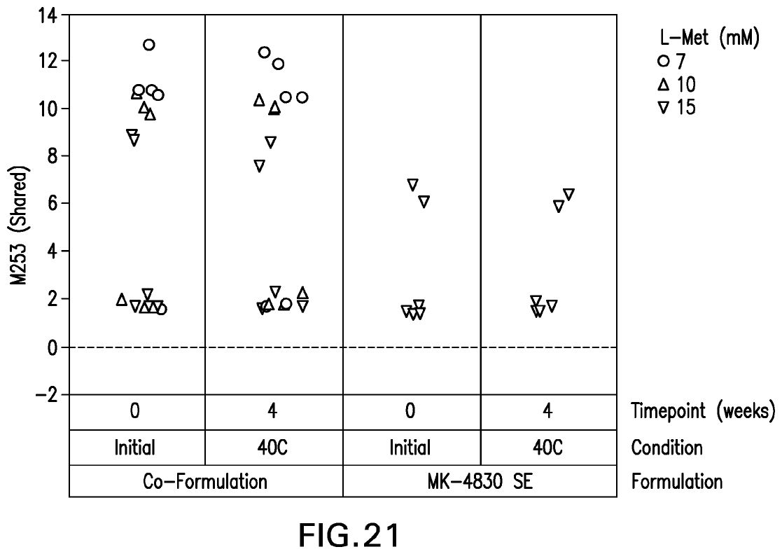

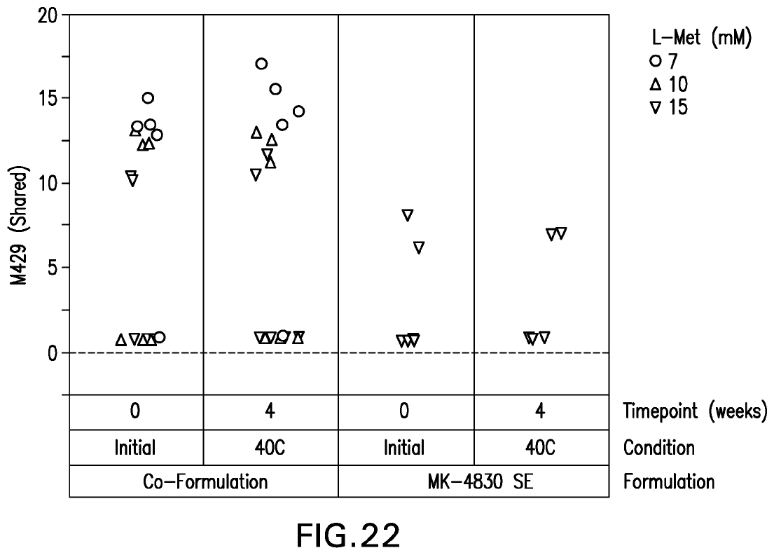

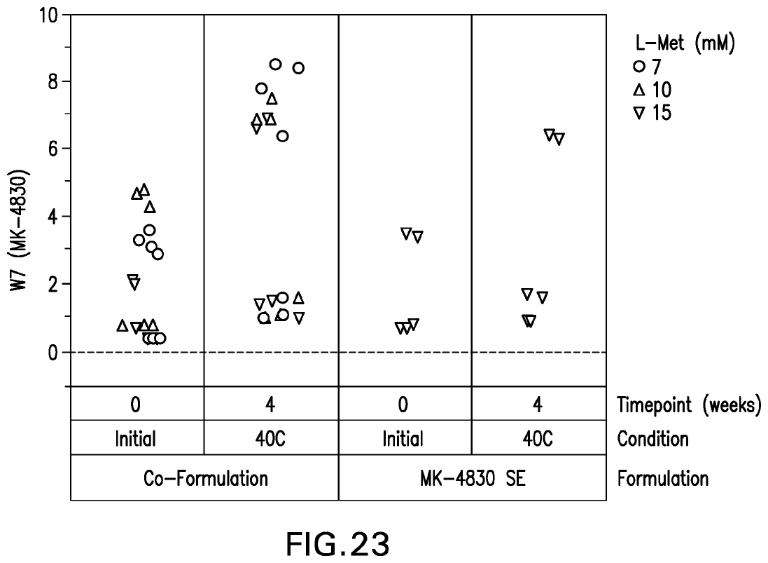

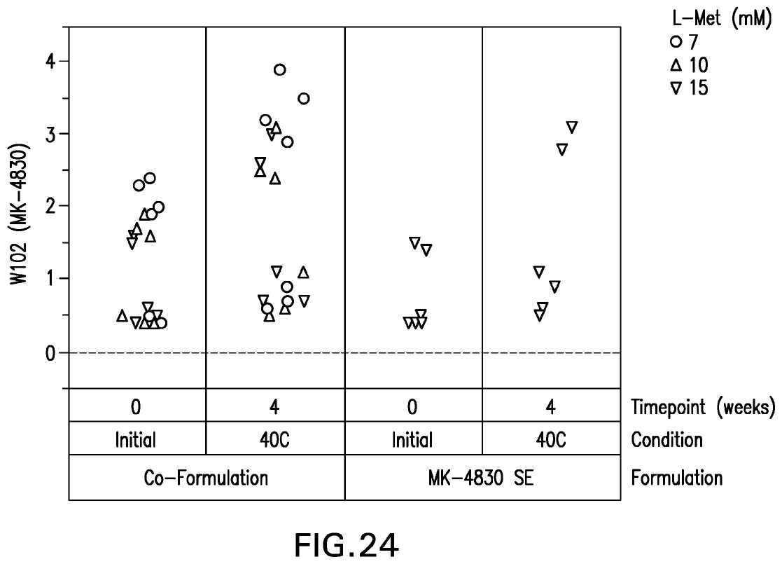

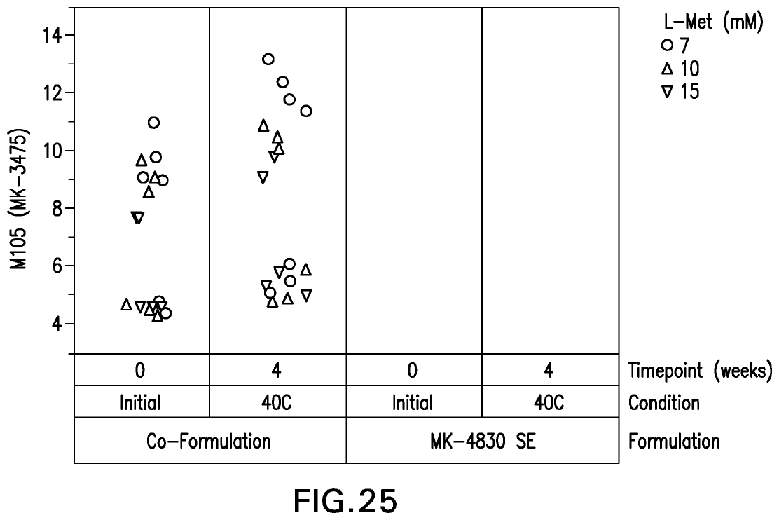

FIGS. 21-25 depicts peptide mapping (PTM) for the single entity and coformulations of, at initial and 40° C. conditions. Data for the key responses for PTM were collected at initial and 4 weeks at 40° C. for the co-formulation and anti-ILT4 SE formulation for a subset of conditions. A statistical model was fitted for all the PTM responses with main effects and two-way interactions with the 2 stability time points at 40° C. condition. Reduced Peptide Mapping was used to monitor the M105 oxidation for anti-PD-1 and multiple oxidation hotspots for the anti-ILT4 monoclonal antibody within anti-ILT4/anti-PD-1 coformulations. The data for PTM support the final formulations chosen for co-formulation and anti-ILT4 SE formulation. Specifically, FIG. 21 depicts M253 (Shared), FIG. 22 depicts M429 (Shared), FIG. 23 depicts W7 (anti-ILT4), FIG. 24 depicts W102 (anti-ILT4), and FIG. 25 depicts M105 (pembrolizumab).

M105 oxidation for anti-PD-1 monoclonal antibody component is a known liability from other pembrolizumab (anti-PD-1 monoclonal antibody) co-formulated product experience and the impact of increasing L-Methionine (15 mM for CF 1) was evaluated by monitoring changes in M105 residue on stability. FIG. 25 depicts an increasing trend for all the residues for both CF 1 and CF 2 on stability at 40° C. for 6 months. CF 1 (15 mM L-methionine) showed a marginal reduction in M105 oxidation at 3 months and 6 months at 40° C. compared to the CF 2 (10 mM L-methionine). A slight increase in M105 residue was observed at 25° C. after 6 months and 5° C. after 12 months, however, the slope of the increase is low, indicating minimal impact to stability at the recommended storage condition at 5° C. Similar, stability trend was observed for oxidation of W102 and W7 residues anti-ILT4 monoclonal antibody in CF 1 and CF 2, where oxidation increased at 40° C. after 6 months and no measurable changes at recommended storage condition of 5° C. after 12 months.

### Soluble Aggregates (UP-SEC)

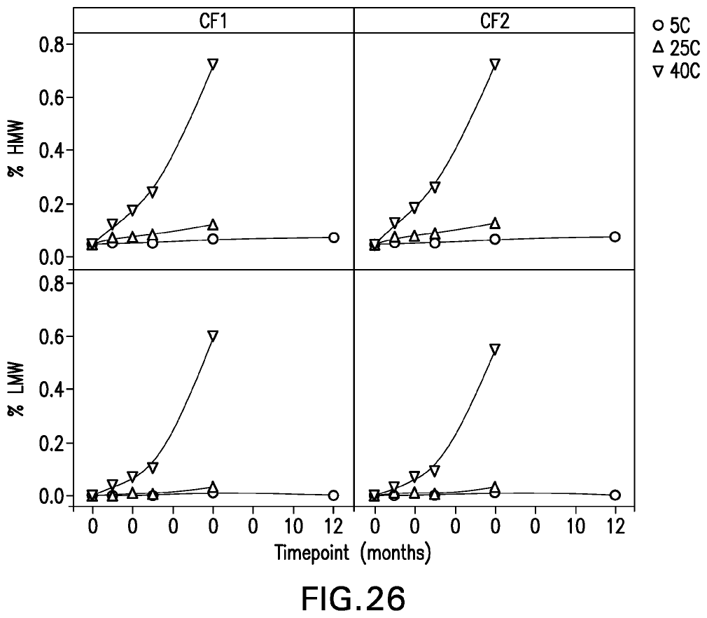

As shown in FIG. 26, the high molecular weight (HMW) and low molecular weight (LMW) species as determined by UP-SEC in coformulation 1 (CF1) and coformulation 2 (CF 2) when stored at 5° C. (circle) for 12 months, 25° C. (triangle pointing up) and 40° C. (triangle pointing down) for 6 months. Lines correspond to a third-order polynomial fit, and it is only used for guidance in the figure. Both formulations showed an increasing trend in HMW on stability at 40° C. storage over 6 months. There was a slight increase in HMW at 25° C. over 6 months. Aggregate content did not change at 5° C. for up to 12 months, indicating sufficient physical stability at recommended storage conditions. No measurable difference was observed in soluble aggregates between co-formulation prototypes with L-Methionine content varying from 10 mM to 15 mM.

### Charge Variants (HP-IE)

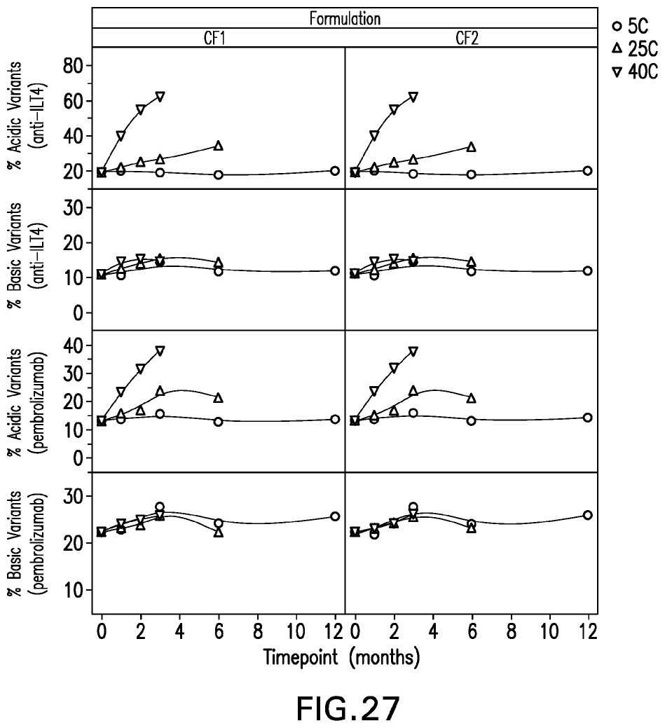

As shown in FIG. 27, the anti-ILT4 and pembrolizumab charged variants as determined by IEX in CF 1 and CF 2 when stored at 5° C. (circle) for 12 months, 25° C. (triangle pointing up) and 40° C. (triangle pointing down) for 6 months. Lines correspond to a third-order polynomial fit, and it is only used for guidance in the figure.

CF 1 and CF 2 were evaluated for chemical degradation by IEX-Combination method, where the two single entities (anti-ILT4 monoclonal antibody and pembrolizumab), were separated for main peak and acidic/basic charged variants. Both formulations showed a stability-based decrease in main-peak and increase in acidic and basic variants at 40° C. Slight decrease in main peak and increased charge variants was observed at 25° C. for 6 months, while a very minimal change in charge species was observed at 5° C. for 12 months. No measurable difference in chemical degradation was observed in the co-formulation prototypes with increasing L-Methionine content to 15 mM.

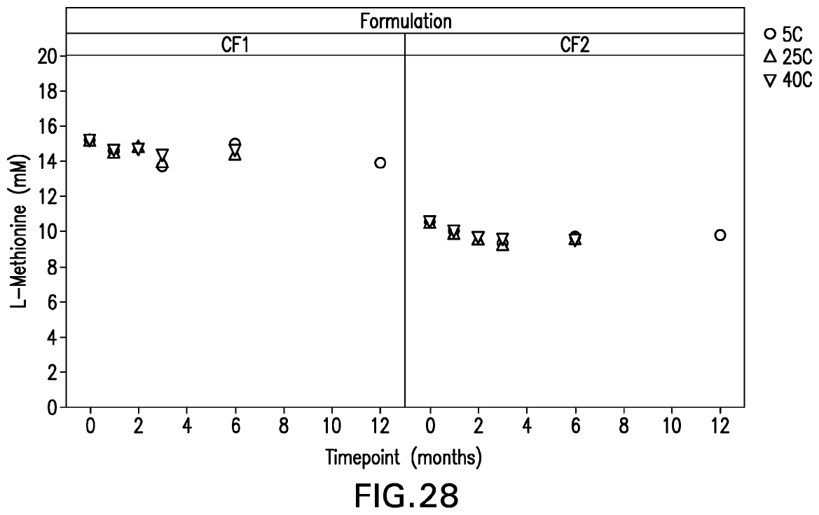

As shown in FIG. 28, the total L-Methionine content in CF 1 and CF 2 when stored at 5° C. (circle) for 12 months, 25° C. (triangle pointing down) and 40° C. (triangle pointing up) for 6 months.

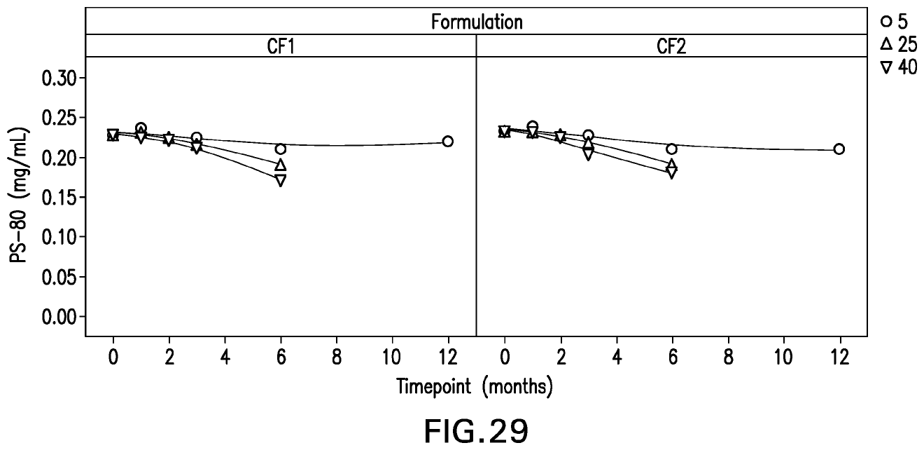

As shown in FIG. 29, the total PS-80 content in CF1 and CF2 when stored at 5° C. (circle) for 12 months, 25° C. (triangle pointing up) and 40° C. (triangle pointing down) for 6 months. Lines correspond to a third-order polynomial fit, and it is only used for guidance in the figure. A temperature-dependent drop in PS-80 was observed at 25° C. and 40° C. after 6 months storage. Within Analytical assay variability, no change was observed in PS-80 content for CF 1 and CF 2 after 12 months storage at 5° C.

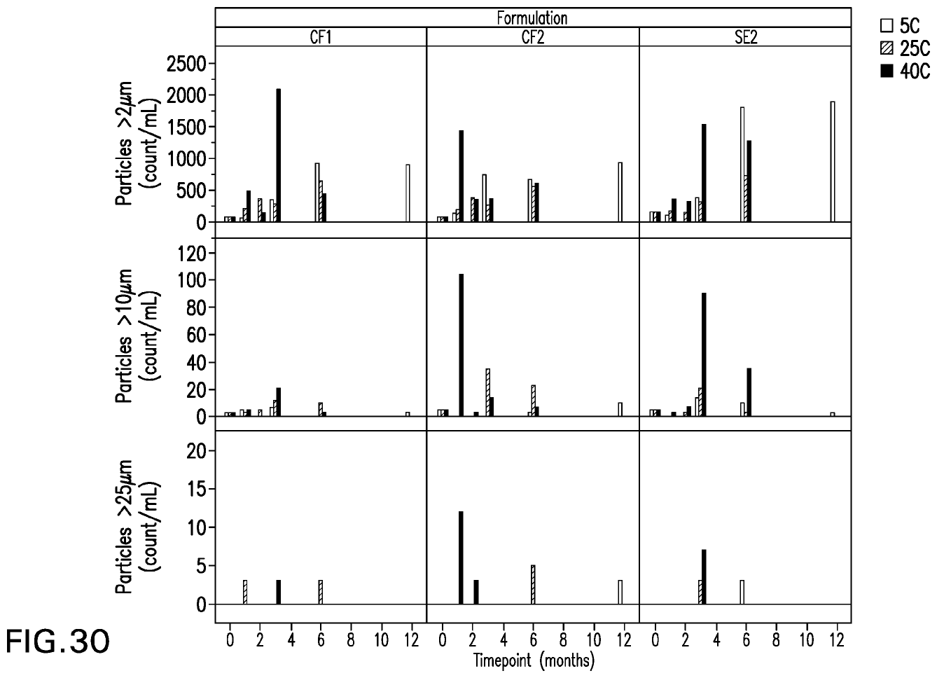

FIG. 30 shows sub-visible particle count in CF1 and CF2 when stored at 5° C. (light gray) for 12 months, 25° C. (dark gray) and 40° C. (black) for 6 months. A stability temperature-dependent increase in the particle count was observed for both CF 1 and CF 2 formulations on stability. Overall, the particle count for both smaller particles (≥2 μm and −5 μm) and larger particles (≥10 μm and ≥25 μm) remained low on stability.

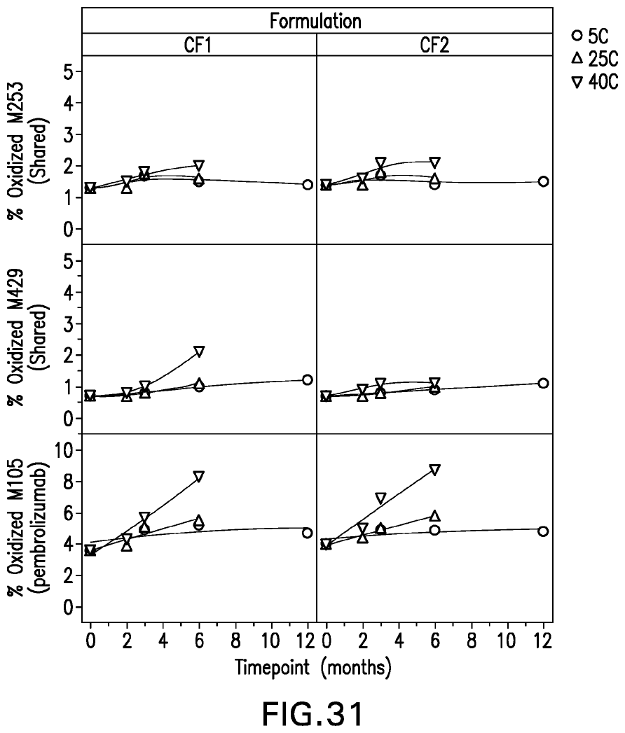

FIG. 31 shows oxidation of M105 (anti-PD-1), M429 (shared residue) and M253 (shared residue) as determined by Reduced Peptide Mapping CF 1 and CF 2 when stored at 5° C. (circle) for 12 months, 25° C. (triangle pointing up) and 40° C. (triangle pointing down) for 6 months. Lines correspond to a third-order polynomial fit, and it is only used for guidance in the figure.

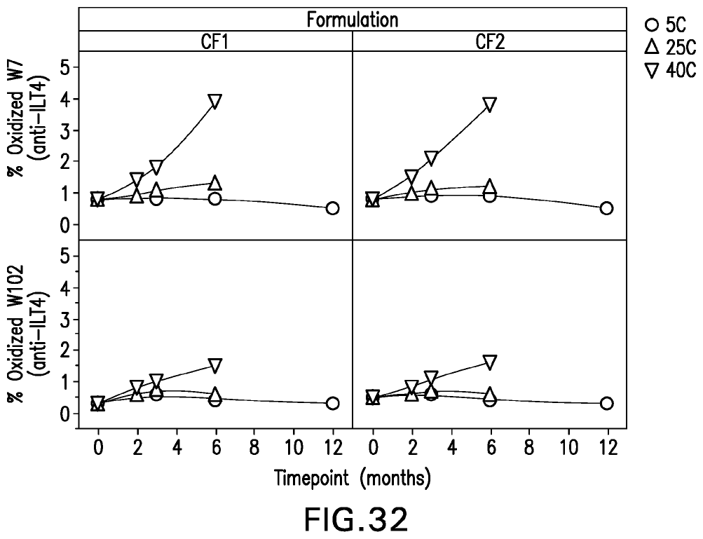

FIG. 32 shows oxidation of W102 (anti-ILT4 monoclonal antibody) and W7 (anti-ILT4 monoclonal antibody) as determined by Reduced Peptide Mapping CF 1 and CF 2 when stored at 5° C. (circle) for 12 months, 25° C. (triangle pointing up) and 40° C. (triangle pointing down) for 6 months. Lines correspond to a third-order polynomial fit, and it is only used for guidance in the figure.

Formulation studies with fixed dose ratios of anti-ILT4 monoclonal antibody and anti-PD-1 monoclonal antibody indicated the feasibility to develop an anti-ILT4/anti-PD-1 coformulation to accommodate fixed dose of 800 mg of anti-ILT4 monoclonal antibody with of 200 mg of anti-PD-1 monoclonal antibody.

Based on the Design-of-Experiment and pre-probe stability studies for co-formulation, the following conclusions were made:
anti-ILT4/anti-PD-1 coformulation has a pH of 5.5 which was confirmed to be optimal for stability.
L-Histidine buffer (10 mM) was confirmed as an acceptable buffer system for anti-ILT4/anti-PD-1 coformulation.
Sucrose at 70 mg/mL concentration was supplemented in the 10 mM L-histidine pH 5.5 matrix as thermal stabilizer.
PS80 at 0.25 mg/mL was selected as the surfactant of choice based on its ability to improve stability against agitation-induced stress.
L-methionine at 2.24 mg/mL (15 mM) was added as an antioxidant to minimize oxidation under light stress.

Formulation feasibility (Design of experiment) studies evaluated the thermal stability of anti-ILT4/anti-PD-1 coformulation at 5° C. for 12 weeks and at and 40° C. for 4 weeks, after subjecting the samples to different stress conditions (process and formulation factors).

### Example 9: Manufacturing and DP Image Considerations

### Diluent Selection:

For anti-ILT4/anti-PD-1 coformulation, to maintain the recommended L-Methionine (15 mM or 2.24 mg/mL) and PS-80 (0.25 mg/mL) levels in the final composition, a process diluent will be added during the drug product manufacturing process. A process diluent containing 0.29 mg/mL L-histidine, 1.71 mg/mL L-histidine monohydrochloride monohydrate, 17.46 mg/mL L-methionine, 70 mg/mL sucrose and 0.45 mg/mL polysorbate 80 in Water for Injection, was selected as optimal composition based on the range of experience of the previous co-formulation manufacturing at the DP manufacturing site (Carlow). To maintain the drug-product ratio and to accommodate the diluent in appropriate fill-volume, the final ratio was optimized as 8:1:4 (v:v:v) of anti-ILT4 monoclonal antibody:Diluent:anti-PD-1 monoclonal antibody.

### Fill Volume:

The final fill volume was optimized based on following considerations:
1. Dilution to ensure the ratio of 8:1:4 of anti-ILT4:Diluent:anti-PD-1 is maintained in the final DP image
2. The recoverable fill-volume is rounded to appropriate decimal place to enable healthcare professional can measure DP volume accurately using calibration of the syringe (e.g., 26.00 ml is preferred volume to withdraw using 30 mL syringe against 26.7 mL)

The final composition 30.8 mg/mL anti-ILT4 monoclonal antibody, 7.7 mg/mL of anti-PD-1 monoclonal antibody, 0.29 mg/mL L-histidine 1.71 mg/mL L-histidine monohydrochloride monohydrate, 2.24 mg/mL L-methionine, 70 mg/mL sucrose, 0.25 mg/mL polysorbate 80 at pH 5.5 was found to be stable under refrigerated conditions (Table 12).

The stability of the final drug product is assessed at the storage condition of 5° C.±3° C., at the accelerated storage condition of 25° C./60% RH ending after six months, and at the stressed condition of 40° C./75% RH ending after six months.

Tables 13-15 below summarize the stability data for the co-formulation. There were no discernable changes observed for the final Drug product on stability at recommended storage condition of 5C at from any of the assays. Test results for visible particles under all conditions evaluated to date are reported as “Liquid essentially free from visible particles”, which complies with the acceptance criteria. The observations do not change as a function of storage condition or time. Potency stability data at the 5° C., 25° C., and 40° C. storage conditions is captured in Table 13-15. There is a decrease over the first 6 months across the accelerated condition of 40° C. At the stressed condition of 25° C. and the recommended condition of 5° C., minimal changes in potency are seen. A small increase in concentration is seen at the stressed condition at 6M for anti-PD-1 monoclonal antibody component. No other changes were observed in the protein concentration across all time points and conditions.

TABLE 13
Composition of anti-ILT4/anti-PD-1 coformulation Drug product

| | | Concentration | Quantity |
|---|---|---|---|
| Ingredient | Function | (mg/mL)^{a} | (mg/vial) |
| anti-ILT4 | Active | 30.8 | 800 |
| monoclonal | Pharmaceutical | | |
| antibody | Ingredient | | |
| pembrolizumab | Active | 7.7 | 200 |
| | Pharmaceutical | | |
| | Ingredient | | |
| L-Histidine | Buffer | 0.29 | 7.54 |
| L-Histidine | Buffer | 1.71 | 44.46 |
| Monohydrochloride | | | |
| Monohydrate | | | |
| L-Methionine | Stabilizer | 2.24 | 58.24 |
| Sucrose | Stabilizer/ | 70 | 1820 |
| | Tonicity | | |
| | Modifier | | |
| Polysorbate 80 | Surfactant | 0.25 | 6.5 |
| Water for Injection | Solvent | q.s. to 1.0 mL | q.s. to 26.0 mL^{b} |

^{a}excess fill of 1.0 mL is provided to ensure the recovery of the label claim of 800 mg/vial of anti-ILT4 monoclonal antibody and 200 mg/vial of anti-PD-1 monoclonal antibody
^{b}q.s. = quantum sufficit, the quantity that is sufficient; Formulation pH is 5.5

Studies were conducted to justify the amount of the PS-80 in the final composition to protect against Freeze-thaw (F/T) and Agitation induced particle-formation and aggregation. Freeze-thaw (F/T) and Agitation stress studies were conducted on the selected formulation composition listed in Table 14. As shown below, two F/T excursion conditions were studied, a. A temperature excursion study consisting of up to five cycles of temperature exposure alternating between −40° C. and 25° C.; and b. A temperature excursion study consisting of up to five cycles of temperature exposure alternating between −80° C. and 25° C.

TABLE 14
Freeze-Thaw (F/T) Study

| | | 1X F/T | 3X F/T | 5X F/T | 1X F/T | 3X F/T | 5X F/T |
|---|---|---|---|---|---|---|---|
| | | (−40° C. | (−40° C. | (−40° C. | (−80° C. | (−80° C. | (−80° C. |
| | | to | to | to | to | to | to |
| Assay | Control | 25° C.) | 25° C.) | 25° C.) | 25° C.) | 25° C.) | 25° C.) |
| pH | 5.5 | 5.5 | 5.5 | 5.5 | 5.5 | 5.5 | 5.5 |
| Turbidity (OD_{350-500 nm}) | 0.095 | 0.103 | 0.100 | 0.117 | 0.095 | 0.094 | 0.106 |
| anti- | 31.23 | 33.08 | 33.22 | 33.91 | 31.96 | 32.22 | 33.36 |
| ILT4concentration | | | | | | | |
| (mg/mL) | | | | | | | |
| anti-PD-1 | 7.38 | 8.56 | 8.52 | 8.75 | 8.24 | 8.30 | 8.63 |
| concentration | | | | | | | |
| (mg/mL) | | | | | | | |

Purity by UP-SEC

| | | | | | | | |
|---|---|---|---|---|---|---|---|
| % HMW | 0.55 | 0.54 | 0.54 | 0.54 | 0.55 | 0.54 | 0.54 |
| % Monomer | 99.4 | 99.5 | 99.5 | 99.5 | 99.4 | 99.5 | 99.5 |
| % LMW | 0.00 | 0.00 | 0.00 | 0.01 | 0.00 | 0.00 | 0.00 |

Charge Variants by HP-IEX
anti-ILT4

| | | | | | | | |
|---|---|---|---|---|---|---|---|
| % Acidic Variants | 16.0 | 15.6 | 15.7 | 14.7 | 15.4 | 15.4 | 15.8 |
| % Main | 67.8 | 68.4 | 68.3 | 69.8 | 68.2 | 68.6 | 68.1 |
| % Basic Variants | 16.2 | 16.0 | 16.0 | 15.6 | 16.3 | 16.0 | 16.1 |

anti-PD-1

| % Acidic Variants | 15.6 | 15.7 | 15.1 | 13.4 | 15.4 | 14.6 | 14.8 |
|---|---|---|---|---|---|---|---|
| % Main | 62.8 | 63.0 | 63.8 | 64.8 | 63.2 | 64.6 | 63.6 |
| % Basic Variants | 21.6 | 21.3 | 21.1 | 21.7 | 21.3 | 20.8 | 21.6 |
| PS-80 (mg/mL) | 0.23 | 0.19 | 0.18 | 0.13 | 0.22 | 0.22 | 0.15 |
| L-Methionine (mM) | 14.7 | 14.7 | 14.4 | 13.9 | 14.8 | 15.3 | 13.6 |
| Particulate matter by MFI | | | | | | | |
| (particle count per mL) | | | | | | | |
| ≥2 μm | 165 | 69 | 78 | 170 | 293 | 108 | 113 |
| ≥5 μm | 42 | 7 | 9 | 8 | 43 | 17 | 5 |
| ≥10 μm | 11 | 1 | 2 | 1 | 9 | 3 | 2 |
| ≥25 μm | 1 | 0 | 1 | 0 | 0 | 1 | 0 |

* The data-set for the Control is as reported in Table A1

As shown in Table 15, an agitation stress study under ambient conditions where the drug product vials were agitated at a speed of 300 rpm for 3 and 7 days. Drug product vials also under ambient conditions but not subject to agitation served as controls.

TABLE 15
Agitation Stress Study

| | Agitation Control | | Agitation Stress | |
|---|---|---|---|---|
| Assay | 3 Day | 7 Day | 3 Day | 7 Day |
| pH | 5.4 | 5.5 | 5.5 | 5.5 |
| Turbidity (OD_{350-500 nm}) | 0.095 | 0.093 | 0.095 | 0.095 |
| anti-ILT4concentration (mg/mL) | 31.07 | 30.92 | 30.85 | 31.17 |
| anti-PD-1 concentration (mg/mL) | 7.36 | 7.40 | 7.36 | 7.40 |

Purity by UP-SEC

| | | | | |
|---|---|---|---|---|
| % HMW | 0.56 | 0.58 | 0.57 | 0.58 |
| % Monomer | 99.4 | 99.4 | 99.4 | 99.4 |
| % LMW | 0.00 | 0.00 | 0.00 | 0.00 |

Charge Variants by HP-IEX
anti-ILT4

| | | | | |
|---|---|---|---|---|
| % Acidic Variants | 15.5 | 15.8 | 15.6 | 15.6 |
| % Main | 68.4 | 67.9 | 68.2 | 68.3 |
| % Basic Variants | 16.1 | 16.3 | 16.2 | 16.2 |

anti-PD-1

| | | | | |
|---|---|---|---|---|
| % Acidic Variants | 15.9 | 17.1 | 15.7 | 16.4 |
| % Main | 63.1 | 62.5 | 62.9 | 62.7 |
| % Basic Variants | 20.9 | 20.4 | 21.4 | 20.9 |
| PS-80 (mg/mL) | 0.22 | 0.22 | 0.23 | 0.22 |
| L-Methionine (mM) | 15.3 | 15.4 | 15.3 | 14.9 |

Particulate matter by MFI (particle count per mL)

| | | | | |
|---|---|---|---|---|
| ≥2 μm | 185 | 110 | 132 | 148 |
| ≥5 μm | 35 | 10 | 31 | 23 |
| ≥10 μm | 9 | 4 | 10 | 3 |
| ≥25 μm | 0 | 1 | 1 | 0 |

There was no change in turbidity, high molecular weight species, charge variants, and subvisible particulate count by MFI upon freeze-thaw exposure between −40° C. and 25° C. for up to five freeze-thaw cycles (5×FIT). A decrease in PS-80 and L-methionine content was observed at 5× F/T for both −40° C. to 25° C. and −80° C. to 25° C. Results are captured in Table 20.

There was no change in turbidity, high molecular weight species, charge variants, L-methionine content, and PS-80 content upon agitation up to 7 days under ambient temperature conditions at 300 rpm. A small increase in subvisible particle count was observed in the MFI results for the 7-day agitated drug product compared to the 7-day control for ≥2 m and ≥5 μm particles. Results are captured in Table 21.

The data from the Freeze-thaw stress and Agitation stress studies do not show any significant changes in the attributes indicating that the selected level of S-80 (0.25 mg/mL) in the final co-formulation composition is sufficient to protect against Free-thaw and agitation-induced particle-formation and aggregation

### Example 10: Clarity and Degree of Opalescence of Drug Product

Test results for clarity and degree of opalescence of drug product are captured in Tables 16-18. Results from the color analysis of drug product under all conditions evaluated to date meet the acceptance criteria of “Not more intense than reference Y5.” No appreciable changes in PS-80 content were observed at the 5° C. condition through nine months The 25° C. and 40° C. conditions trend lower through six months but are within the specification.

No change in L-methionine was observed across conditions and time points throughout the study.

Nine months of HP-IEX data showed no substantial changes in acidic variants after storage at the proposed storage condition of 5° C. At the 25° C. condition, there are slight increases in the % Acidic variants with a significant increase seen at 40 C through six months. No changes are observed in % Basic variants across all conditions of the study for anti-PD-1 monoclonal antibody with a decrease seen at the 40 C condition for anti-ILT4/anti-PD-1 coformulation through six months. A marked decrease in % Main is seen at 40 C for anti-ILT4/anti-PD-1 coformulation with a smaller decrease observed for anti-PD-1 monoclonal antibody through six months. A minor decrease was also observed at 25° C. for anti-ILT4/anti-PD-1 coformulation. No change was observed for the remaining conditions through nine months for either component. No change was observed across conditions and time points for % Combined variants for the formulations tested, anti-PD-1 monoclonal antibody % Main decreases slightly through six months with no changes for the remaining conditions for research probe. No change was observed for % Mixed Basic variants across conditions through nine months for formulations tested.

No variation is observed in % Monomer or % High molecular weight species at 5 C and 25 C for the formulations tested through nine months. At 40° C., a gradual decrease is seen through six months with results falling below specification at six months for % monomer. Results exceed the upper specification limit for % high molecular weight species at 40° C. after three months.

An increase in the % low molecular weight species is observed which falls below the specification limit after two months for the 40° C. condition with no changes observed at the 5 C and 25° C. condition for the formulations tested.

No appreciable change was observed in % intact IgG at the 5 C and 25 C condition for the formulations tested. Results from the 40 C condition show a decline approaching the six month time point with data falling below the specification after three months.

The formulations tested showed no change observed for % Heavy Chain+Light Chain at the recommended storage condition of 5 C and the stressed condition of 25 C. At 40 C, the % Heavy Chain+Light Chain results decrease and fall below specification after three months. No changes were observed in the duration of the study across conditions and time points for the formulations tested.

Throughout the study and across conditions, the visible particulate count was within specification for the formulations tested. Results from the full analytical test panel were within the acceptance criteria for up to 9 Months at the recommended storage condition.

TABLE 16
Stability Data for anti-ILT4/anti-PD-1 coformulation at 5° C. ± 3° C.

| | Acceptance | Time Point | | | | | |
|---|---|---|---|---|---|---|---|
| Attributes | Criteria ^{a} | Initial | 1 Month | 2 Months | 3 Months | 6 Months | 9 Months |
| Appearance | Liquid | Liquid | Liquid | Liquid | Liquid | Liquid | Liquid |
| | essentially | essentially | essentially | essentially | essentially | essentially | essentially |
| | free from | free from | free from | free from | free from | free from | free from |
| | visible | visible | visible | visible | visible | visible | visible |
| | particles | particles. | particles. | particles. | particles. | particles. | particles. |
| Clarity and | Report | <Reference | <Reference | <Reference | <Reference | <Reference | <Reference |
| degree of | Results | III | III | standard | Standard | suspension | suspension |
| opalescence | | | | III | III | III | III |
| Color | Not more | Not more | Not more | Not more | Not more | Not more | Not more |
| | intense | intense | intense | intense | intense | intense | intense |
| | than | than | than | than | than | than | than |
| | reference | reference | reference | reference | reference | reference | reference |
| | Y5 | Y7 | Y7 | Y7 | Y7 | Y7 | Y7 |
| Binding | 60%-140% | 98 | 106 | 98 | 93 | 102 | 104 |
| ELISA (anti- | relative | | | | | | |
| ILT4) | potency | | | | | | |
| Binding | 60%-140% | 104 | 83 | 97 | 95 | 95 | 90 |
| ELISA (anti- | relative | | | | | | |
| PD-1) | potency | | | | | | |
| Protein | 26.2-35.4 | 30.8 | 31.9 | 31.7 | 31.9 | 32.7 | 30.7 |
| Concentration | mg/mL | | | | | | |
| (anti-ILT4) | | | | | | | |
| Protein | 6.5-8.9 | 7.7 | 7.9 | 7.8 | 7.9 | 7.9 | 8.1 |
| Concentration | mg/mL | | | | | | |
| (anti-PD-1) | | | | | | | |
| Charge | | | | | | | |
| variants | | | | | | | |
| by HP-IEX | | | | | | | |
| anti-ILT4 | Report | 13.8 | 14.0 | 13.7 | 13.3 | 13.7 | 14.2 |
| Acidic | Results | | | | | | |
| Variants | | | | | | | |
| (X.X %) | | | | | | | |
| anti-ILT4 | Report | 53.9 | 53.9 | 54.1 | 53.9 | 53.1 | 52.3 |
| Main (X.X %) | Results | | | | | | |
| anti-ILT4 | Report | 11.3 | 11.4 | 11.3 | 11.4 | 11.3 | 11.3 |
| Basic 1 | Results | | | | | | |
| (X.X %) | | | | | | | |
| anti-ILT4 | Report | 2.1 | 2.1 | 2.1 | 2.1 | 2.2 | 2.2 |
| Basic 2 | Results | | | | | | |
| (X.X %) ^{b} | | | | | | | |
| anti-ILT4 | Report | 1.5 | 1.5 | 1.4 | 1.6 | 1.8 | 1.9 |
| Basic 3 | Results | | | | | | |
| (X.X %) ^{b} | | | | | | | |
| anti-PD-1 | Report | 3.9 | 3.9 | 3.9 | 4.1 | 4.2 | 4.3 |
| Combined | Results | | | | | | |
| Variants | | | | | | | |
| (X.X %) | | | | | | | |
| anti-PD-1 | Report | 11.6 | 11.6 | 11.6 | 11.7 | 11.6 | 11.8 |
| Main (X.X %) | Results | | | | | | |
| anti-PD-1 | Report | 5.4 | 5.2 | 5.4 | 5.6 | 6.1 | 6.2 |
| Mixed Basic | Results | | | | | | |
| Variants | | | | | | | |
| (X.X %) | | | | | | | |
| anti-PD-1 | Report | 1.8 | 1.8 | 1.8 | 1.9 | 2.0 | 2.0 |
| Acidic 1 | Results | | | | | | |
| (X.X %) ^{b} | | | | | | | |
| anti-PD-1 | Report | 1.1 | 1.1 | 1.1 | 1.1 | 1.1 | 1.1 |
| Basic 1 | Results | | | | | | |
| (X.X %) ^{b} | | | | | | | |
| anti-PD-1 | Report | 2.8 | 2.7 | 2.9 | 2.9 | 3.1 | 3.1 |
| Basic 2 | Results | | | | | | |
| (X.X %) ^{b} | | | | | | | |
| Purity by UP-SEC | | | | | | | |
| Monomer | ≥95.0% | 99.3 | 99.3 | 99.3 | 99.1 | 99.2 | 99.2 |
| High | ≤5.0% | 0.6 | 0.6 | 0.7 | 0.7 | 0.7 | 0.8 |
| Molecular | | | | | | | |
| Weight | | | | | | | |
| Species | | | | | | | |
| (HMW) | | | | | | | |
| Non-reduced | | | | | | | |
| CE-SDS | | | | | | | |
| Intact IgG | ≥90.0% | 98.8 | 98.5 | 98.5 | 98.4 | 98.4 | 98.8 |
| Total Low | ≤10.0% | 1.0 | 1.4 | 1.3 | 1.4 | 1.3 | 0.9 |
| Molecular | | | | | | | |
| Weight | | | | | | | |
| Species | | | | | | | |
| (Total LMW) | | | | | | | |
| Reduced CE-SDS | | | | | | | |
| Heavy | ≥90.0% | 98.3 | 98.6 | 98.6 | 98.6 | 98.7 | 98.4 |
| chain + light | | | | | | | |
| chain | | | | | | | |
| Total | ≤10.0% | 1.7 | 1.4 | 1.4 | 1.4 | 1.3 | 1.6 |
| Impurities | | | | | | | |
| Polysorbate | Report | 0.24 | 0.25 | 0.24 | 0.22 | 0.21 | 0.25 |
| 80 ^{c} | Results | | | | | | |
| | (X.XX | | | | | | |
| | mg/mL) | | | | | | |
| L- | Report | 2.27 | 2.27 | 2.35 | 2.30 | 2.26 | 2.29 |
| Methionine ^{c} | Results | | | | | | |
| | (X.XX | | | | | | |
| | mg/mL) | | | | | | |
| Particulate matter | | | | | | | |
| Particles >=10 um | ≤6000 per | 46 | 0 | 54 | 63 | 73 | 0 |
| | container | | | | | | |
| Particles >=25 um | ≤600 per | 27 | 0 | 0 | 0 | 18 | 0 |
| | container | | | | | | |
| pH | 5.0-6.0 | 5.4 | 5.4 | 5.4 | 5.4 | 5.4 | 5.4 |

^{a} The Acceptance Criteria apply only to material stored at the recommended storage condition.
^{b} The individual species are tracked on stability for information only.

TABLE 17
Stability Data for anti-ILT4/anti-PD-1 coformulation at 25° C./60% Relative Humidity

| | Acceptance | Time Point | | | | | |
|---|---|---|---|---|---|---|---|
| Attributes | Criteria ^{a} | Initial | 2 Weeks | 1 Month | 2 Months | 3 Months | 6 Months |
| Appearance | Liquid | Liquid | Liquid | Liquid | Liquid | Liquid | Liquid |
| | essentially | essentially | essentially | essentially | essentially | essentially | essentially |
| | free from | free from | free from | free from | free from | free from | free from |
| | visible | visible | visible | visible | visible | visible | visible |
| | particles | particles. | particles. | particles. | particles. | particles. | particles. |
| Clarity and | Report | <Reference | <Reference | <Reference | <Reference | <Reference | <Reference |
| degree of | Results | III | III | III | Standard | Standard | Suspension |
| opalescence | | | | | III | III | III |
| Color | Not more | Not more | Not more | Not more | Not more | Not more | Not more |
| | intense | intense | intense | intense | intense | intense | intense |
| | than | than | than | than | than | than | than |
| | reference | reference | reference | reference | reference | reference | reference |
| | Y5 | Y7 | Y7 | Y7 | Y7 | Y7 | Y7 |
| Binding | 60%-140% | 98 | 95 | 107 | 98 | 101 | 101 |
| ELISA (anti- | relative | | | | | | |
| ILT4) | potency | | | | | | |
| Binding | 60%-140% | 104 | 101 | 85 | 93 | 99 | 101 |
| ELISA (anti- | relative | | | | | | |
| PD-1) | potency | | | | | | |
| Protein | 26.2-35.4 | 30.8 | 30.2 | 31.7 | 32.1 | 32.1 | 31.7 |
| Concentration | mg/mL | | | | | | |
| (anti-ILT4) | | | | | | | |
| Protein | 6.5-8.9 | 7.7 | 7.7 | 7.8 | 8.0 | 8.1 | 8.1 |
| Concentration | mg/mL | | | | | | |
| (anti-PD-1) | | | | | | | |
| Charge | | | | | | | |
| variants by | | | | | | | |
| HP-IEX | | | | | | | |
| anti-ILT4 | Report | 13.8 | 14.0 | 15.2 | 16.2 | 18.4 | 24.0 |
| Acidic | Results | | | | | | |
| Variants | | | | | | | |
| (X.X %) | | | | | | | |
| anti-ILT4 | Report | 53.9 | 53.2 | 51.8 | 50.2 | 47.4 | 42.0 |
| Main (X.X %) | Results | | | | | | |
| anti-ILT4 | Report | 11.3 | 11.2 | 11.0 | 10.6 | 10.3 | 9.3 |
| Basic 1 | Results | | | | | | |
| (X.X %) | | | | | | | |
| anti-ILT4 | Report | 2.1 | 2.2 | 2.4 | 2.6 | 2.8 | 3.2 |
| Basic 2 | Results | | | | | | |
| (X.X %) ^{b} | | | | | | | |
| anti-ILT4 | Report | 1.5 | 1.8 | 2.1 | 2.4 | 2.8 | 2.9 |
| Basic 3 | Results | | | | | | |
| (X.X %) ^{b} | | | | | | | |
| anti-PD-1 | Report | 3.9 | 4.2 | 4.4 | 4.9 | 5.6 | 6.7 |
| Combined | Results | | | | | | |
| Variants | | | | | | | |
| (X.X %) | | | | | | | |
| anti-PD-1 | Report | 11.6 | 11.6 | 11.5 | 11.4 | 11.2 | 10.6 |
| Main (X.X %) | Results | | | | | | |
| anti-PD-1 | Report | 5.4 | 5.9 | 6.1 | 6.8 | 7.2 | 7.4 |
| Mixed Basic | Results | | | | | | |
| Variants | | | | | | | |
| (X.X %) | | | | | | | |
| anti-PD-1 | Report | 1.8 | 1.9 | 2.0 | 2.3 | 2.8 | 3.5 |
| Acidic 1 | Results | | | | | | |
| (X.X %) ^{b} | | | | | | | |
| anti-PD-1 | Report | 1.1 | 1.2 | 1.1 | 1.2 | 1.3 | 1.4 |
| Basic 1 | Results | | | | | | |
| (X.X %) ^{b} | | | | | | | |
| anti-PD-1 | Report | 2.8 | 2.8 | 2.9 | 3.1 | 3.1 | 3.1 |
| Basic 2 | Results | | | | | | |
| (X.X %) ^{b} | | | | | | | |
| Monomer | ≥95.0% | 99.3 | 99.2 | 99.2 | 99.1 | 98.9 | 98.8 |
| High | ≤5.0% | 0.6 | 0.7 | 0.7 | 0.8 | 0.9 | 1.1 |
| Molecular | | | | | | | |
| Weight | | | | | | | |
| Species | | | | | | | |
| (HMW) | | | | | | | |
| Non-reduced | | | | | | | |
| CE-SDS | | | | | | | |
| Intact IgG | ≥90.0% | 98.8 | 98.4 | 98.2 | 98.4 | 98.0 | 97.1 |
| Total Low | ≤10.0% | 1.0 | 1.4 | 1.6 | 1.4 | 1.6 | 2.4 |
| Molecular | | | | | | | |
| Weight | | | | | | | |
| Species | | | | | | | |
| (Total LMW) | | | | | | | |
| Reduced CE-SDS | | | | | | | |
| Heavy | ≥90.0% | 98.3 | 98.2 | 98.6 | 98.6 | 98.1 | 98.3 |
| chain + light | | | | | | | |
| chain | | | | | | | |
| Total | ≤10.0% | 1.7 | 1.8 | 1.4 | 1.4 | 1.9 | 1.7 |
| Impurities | | | | | | | |
| Polysorbate | Report | 0.24 | 0.23 | 0.25 | 0.24 | 0.21 | 0.18 |
| 80 ^{c} | Results | | | | | | |
| | (X.XX | | | | | | |
| | mg/mL) | | | | | | |
| L- | Report | 2.27 | 2.25 | 2.25 | 2.36 | 2.31 | 2.25 |
| Methionine ^{c} | Results | | | | | | |
| | (X.XX | | | | | | |
| | mg/mL) | | | | | | |
| Particulate matter | | | | | | | |
| Particles >=10 um | ≤6000 per | 46 | 63 | 46 | 46 | 90 | 36 |
| | container | | | | | | |
| Particles >=25 um | ≤600 per | 27 | 9 | 0 | 0 | 0 | 0 |
| | container | | | | | | |
| Particulate matter | | | | | | | |
| (Subvisible Particles) ^{c} | | | | | | | |
| Particles >=2 μm | Report | 378 | 522 | 559 | 1350 | 765 | 972 |
| | Results | | | | | | |
| Particles >=5 μm | Report | 108 | 126 | 81 | 252 | 225 | 225 |
| | Results | | | | | | |
| pH | 5.0-6.0 | 5.4 | 5.4 | 5.4 | 5.4 | 5.4 | 5.4 |

^{a} The Acceptance Criteria apply only to material stored at the recommended storage condition.
^{b} The individual species are tracked on stability for information only.

TABLE 18
Stability Data for anti-ILT4/anti-PD-1 coformulation at 40° C./75% Relative Humidity

| | Acceptance | Time Point | | | | | |
|---|---|---|---|---|---|---|---|
| Attributes | Criteria ^{a} | Initial | 2 Weeks | 1 Month | 2 Months | 3 Months | 6 Months |
| Appearance | Liquid | Liquid | Liquid | Liquid | Liquid | Liquid | Liquid |
| | essentially | essentially | essentially | essentially | essentially | essentially | essentially |
| | free from | free from | free from | free from | free from | free from | free from |
| | visible | visible | visible | visible | visible | visible | visible |
| | particles | particles. | particles. | particles. | particles. | particles. | particles. |
| Clarity and | Report | <Reference | <Reference | <Reference | <Reference | <Reference | <Reference |
| degree of | Results | III | III | III | standard | Standard | Suspension |
| opalescence | | | | | III | III | IV |
| Color | Not more | Not more | Not more | Not more | Not more | Not more | Not more |
| | intense | intense | intense | intense | intense | intense | intense |
| | than | than | than | than | than | than | than |
| | reference | reference | reference | reference | reference | reference | reference |
| | Y5 | Y7 | Y7 | Y7 | Y7 | Y7 | Y7 |
| Binding | 60%-140% | 98 | 95 | 103 | 90 | 79 | 82 |
| ELISA (anti- | relative | | | | | | |
| ILT4) | potency | | | | | | |
| Binding | 60%-140% | 104 | 99 | 86 | 84 | 94 | 87 |
| ELISA (anti- | relative | | | | | | |
| PD-1) | potency | | | | | | |
| Protein | 26.2-35.4 | 30.8 | 30.3 | 31.4 | 31.0 | 30.9 | 30.7 |
| Concentration | mg/mL | | | | | | |
| (anti-ILT4) | | | | | | | |
| Protein | 6.5-8.9 | 7.7 | 7.8 | 8.1 | 8.2 | 8.3 | 9.2 |
| Concentration | mg/mL | | | | | | |
| (anti-PD-1) | | | | | | | |
| Charge | | | | | | | |
| variants by | | | | | | | |
| HP-IEX | | | | | | | |
| M anti- | Report | 13.8 | 20.6 | 32.1 | 42.2 | 53.3 | 80.8 |
| ILT4 Acidic | Results | | | | | | |
| Variants | | | | | | | |
| (X.X %) | | | | | | | |
| anti-ILT4 | Report | 53.9 | 45.0 | 34.7 | 25.9 | 17.3 | 0.0 |
| Main (X.X %) | Results | | | | | | |
| anti-ILT4 | Report | 11.3 | 10.1 | 8.6 | 7.1 | 4.7 | 0.0 |
| Basic 1 | Results | | | | | | |
| (X.X %) | | | | | | | |
| anti-ILT4 | Report | 2.1 | 3.3 | 4.0 | 4.4 | 5.5 | 5.5 |
| Basic 2 | Results | | | | | | |
| (X.X %) ^{b} | | | | | | | |
| M anti- | Report | 1.5 | 2.6 | 2.4 | 2.1 | 1.5 | 0.0 |
| ILT4 Basic 3 | Results | | | | | | |
| (X.X %) ^{b} | | | | | | | |
| anti-PD-1 | Report | 3.9 | 6.4 | 8.4 | 10.0 | 11.8 | 11.1 |
| Combined | Results | | | | | | |
| Variants | | | | | | | |
| (X.X %) | | | | | | | |
| anti-PD-1 | Report | 11.6 | 10.7 | 9.1 | 7.4 | 5.4 | 3.5 |
| Main (X.X %) | Results | | | | | | |
| anti-PD-1 | Report | 5.4 | 7.2 | 7.2 | 7.3 | 7.5 | 4.7 |
| Mixed Basic | Results | | | | | | |
| Variants | | | | | | | |
| (X.X %) | | | | | | | |
| anti-PD-1 | Report | 1.8 | 3.1 | 4.5 | 5.6 | 6.3 | 5.6 |
| Acidic 1 | Results | | | | | | |
| (X.X %) ^{b} | | | | | | | |
| anti-PD-1 | Report | 1.1 | 1.4 | 1.5 | 1.6 | 1.4 | 0.0 |
| Basic 1 | Results | | | | | | |
| (X.X %) ^{b} | | | | | | | |
| anti-PD-1 | Report | 2.8 | 3.2 | 3.3 | 3.6 | 4.5 | 4.7 |
| Basic 2 | Results | | | | | | |
| (X.X %) ^{b} | | | | | | | |
| Purity by | | | | | | | |
| UP-SEC | | | | | | | |
| Monomer | ≥95.0% | 99.3 | 98.8 | 98.2 | 97.1 | 94.5 | 87.4 |
| High | ≤5.0% | 0.6 | 1.0 | 1.4 | 2.1 | 3.7 | 9.2 |
| Molecular | | | | | | | |
| Weight | | | | | | | |
| Species | | | | | | | |
| (HMW) | | | | | | | |
| Non-reduced | | | | | | | |
| CE-SDS | | | | | | | |
| Intact IgG | ≥90.0% | 98.8 | 97.8 | 96.9 | 94.9 | 91.5 | 86.7 |
| Total Low | ≤10.0% | 1.0 | 1.9 | 2.8 | 4.9 | 7.8 | 11.9 |
| Molecular | | | | | | | |
| Weight | | | | | | | |
| Species | | | | | | | |
| (Total LMW) | | | | | | | |
| Reduced CE-SDS | | | | | | | |
| Heavy | ≥90.0% | 98.3 | 97.5 | 96.5 | 96.3 | 94.1 | 89.1 |
| chain + light | | | | | | | |
| chain | | | | | | | |
| Total | ≤10.0% | 1.7 | 2.5 | 3.5 | 3.7 | 5.9 | 10.9 |
| Impurities | | | | | | | |
| Polysorbate | Report | 0.24 | 0.23 | 0.23 | 0.22 | 0.20 | 0.17 |
| 80 ^{c} | Results | | | | | | |
| | (X.XX | | | | | | |
| | mg/mL) | | | | | | |
| L- | Report | 2.27 | 2.25 | 2.26 | 2.34 | 2.29 | 2.34 |
| Methionine ^{c} | Results | | | | | | |
| | (X.XX | | | | | | |
| | mg/mL) | | | | | | |
| Particulate matter | | | | | | | |
| Particles >=10 um | ≤6000 per | 46 | 46 | 27 | 36 | 171 | 54 |
| | container | | | | | | |
| Particles >=25 um | ≤600 per | 27 | 0 | 0 | 0 | 36 | 9 |
| | container | | | | | | |
| Particulate matter | | | | | | | |
| (Subvisible Particles) ^{c} | | | | | | | |
| Particles >=2 μm | Report | 378 | 252 | 486 | 2619 | 1971 | 3205 |
| | Results | | | | | | |
| Particles >=5 μm | Report | 108 | 81 | 90 | 189 | 505 | 802 |
| | Results | | | | | | |
| pH | 5.0-6.0 | 5.4 | 5.4 | 5.4 | 5.4 | 5.5 | 5.4 |

^{a} The Acceptance Criteria apply only to material stored at the recommended storage condition.
^{b} The individual species are tracked on stability for information only.

### Example 11: Ion Exchange (IEX) Method to Measure Acidic Species of Anti-PD-1 Antibodies

For the IEX method, using a Waters Alliance LC system (Milford, MA, U.S.A.), the Thermo Scientific's ProPac WCX-10 (p/n: 054993, particle size 10 urn, diameter 4 mm, length 250 mm) was chosen with a loading of 80 μg sample. Mobile Phase (A) 24 mM MES pH 6.1 with 4% acetonitrile, and mobile phase (B) 20 mM sodium phosphate, 95 mM NaCl pH 8.0 with 4% acetonitrile was used as a non-linear, sigmoidal shape, pH gradient, and the separation was monitored over 34 min with a flow rate of 0.5 mL min-1, with the column temperature being 35° C. The gradient used was: 22%-22% B for 0-0.6 min; 22%-29% for 0.6-15.0 min; 29%-70% B for 15.0-30.0 min; 70%-100% B for 30.0-30.5 min; and 100%-100% from 30.5-33.0 min. Mobile phase (C) 10 mM CHES pH 8.0, 40 mM Tris, 15 mM EDTA, 200 mM NaCl, and 4% acetonitrile was used to strip the column at 0.5 mL min-1 from 33.1-34.0 min, followed by re-equilibration with 22% B from 34.5-44.5 min at 1.0 mL min-1. From 44.5-45 min, the flowrate was reduced to 0.5 mL min-1. The elution was monitored at 280 nm for the detection of peaks. The assay variability was determined to be within 1%.

U.S. provisional application No. 63/143,461 is incorporated by reference in its entirety. All references cited herein are incorporated by reference to the same extent as if each individual publication, database entry (e.g., GenBank sequences or GeneID entries), patent application, or patent, was specifically and individually indicated to be incorporated by reference each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A formulation comprising:
(i) about 10 mg/mL to about 200 mg/mL of an anti-human immunoglobulin-like transcript 4 (“anti-ILT4”) antibody or antigen-binding fragment thereof;
(ii) about 10 mg/mL to about 200 mg/mL of an anti-PD-1 antibody or antigen-binding fragment thereof,
(iii) about 5 mM to about 20 mM of a buffer;
(iv) about 6% to about 8% weight/volume (w/v) of a non-reducing sugar;
(v) about 0.01% to about 0.10% (w/v) of a non-ionic surfactant; and
(vi) about 1 mM to about 20 mM of an anti-oxidant,
wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21),
wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 102,
wherein the buffer is an L-histidine buffer, an acetate buffer, or a citrate buffer,
wherein the non-reducing sugar is a disaccharide,
wherein the non-ionic surfactant is polysorbate 20 or polysorbate 80, and
wherein the antioxidant is methionine or a pharmaceutically acceptable salt thereof.

2. The formulation of claim 1, comprising 40-60 mg/mL of the anti-ILT4 antibody; 15-35 mg/mL of the anti-PD-1 antibody; about 7% sucrose or trehalose; about 0.025% polysorbate 80 or polysorbate 20; about 3-30 mM L-histidine buffer at pH about 5.0-6.5; and about 5-20 mM methionine or a pharmaceutically acceptable salt thereof.

3-7. (canceled)

8. A formulation comprising:
(i) about 10 mg/mL to about 200 mg/mL of an anti-ILT4 antibody (“anti-ILT4”) or antigen-binding fragment thereof;
(ii) about 10 mg/mL to about 200 mg/mL of an anti-PD-1 antibody or antigen-binding fragment thereof,
(iii) about 5 mM to about 20 mM L-histidine buffer;
(iv) about 6% to about 8% weight/volume (w/v) sucrose;
(v) about 0.01% to about 0.10% (w/v) polysorbate 80; and
(vi) about 1 mM to about 20 mM L-methionine,
wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21); and wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 102, or a pharmaceutically acceptable salt thereof.

9. The formulation of claim 8, comprising about 8 mM to about 12 mM L-histidine buffer, about 5 mM to about 10 mM L-methionine, about 0.01% to about 0.05% (w/v) polysorbate 80, about 10 mg/mL to about 150 mg/mL of the anti-ILT4 antibody or antigen-binding fragment thereof, and/or about 10 mg/mL to about 150 mg/mL of the anti-PD-1 antibody or antigen-binding fragment thereof.

10-15. (canceled)

16. The formulation of claim 1, comprising about 25 mg/mL of the anti-ILT4 antibody or antigen-binding fragment thereof, about 10 mg/mL of the anti-PD-1 antibody or antigen binding fragment thereof, about 10 mM L-histidine buffer, about 7% w/v sucrose, about 0.025% polysorbate 80, and about 10 mM L-methionine.

17. The formulation of claim 8, comprising about 50 mg/mL of the anti-ILT4 antibody or antigen-binding fragment thereof, about 25 mg/mL of the anti-PD-1 antibody or antigen binding fragment thereof, about 10 mM L-histidine buffer, about 7% w/v sucrose, about 0.025% polysorbate 80, and about 10 mM L-methionine.

18. The formulation of claim 1, comprising about 75 mg/mL of the anti-ILT4 antibody or antigen-binding fragment thereof, about 50 mg/mL of the anti-PD-1 antibody or antigen binding fragment thereof, about 10 mM L-histidine buffer, about 7% w/v sucrose, about 0.025% polysorbate 80, and about 10 mM L-methionine.

19. The formulation of claim 8, comprising about 100 mg/mL of the anti-ILT4 antibody or antigen-binding fragment thereof, about 75 mg/mL of the anti-PD-1 antibody or antigen binding fragment thereof, about 10 mM L-histidine buffer, about 7% w/v sucrose, about 0.025% polysorbate 80, and about 10 mM L-methionine.

20. The formulation of claim 1, comprising about 125 mg/mL of the anti-ILT4 antibody or antigen-binding fragment thereof, about 100 mg/mL of the anti-PD-1 antibody or antigen binding fragment thereof, about 10 mM L-histidine buffer, about 7% w/v sucrose, about 0.025% polysorbate 80, and about 10 mM L-methionine.

21. The formulation of claim 1, wherein the formulation has a pH of about 5.0 to about 6.8.

22-23. (canceled)

24. A formulation of an anti-human immunoglobulin-like transcript 4 (“anti-ILT4”) antibody or antigen-binding fragment thereof, comprising:
(i) about 50 mg/mL to about 100 mg/mL of the anti-ILT4 antibody or antigen-binding fragment thereof,
(ii) about 10 mg/mL to about 200 mg/mL of an anti-PD-1 antibody or antigen-binding fragment thereof,
(iii) about 10 mM L-histidine buffer, pH about 5.5;
(iv) about 7% weight/volume (w/v) sucrose;
(v) about 0.025% (w/v) polysorbate 80; and
(vi) about 10 mM L-methionine,
wherein the anti-ILT4 antibody or antigen binding fragment thereof comprises a heavy chain variable domain comprising: CDR-H1: GYYWS (SEQ ID NO: 16), CDR-H2: EINHXGSTNYNPSLKS wherein X is S or A (SEQ ID NO: 17), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX1X2NRPS, wherein X1 is N, Q, E or D and X2 is S or A (SEQ ID NO: 20), and CDR-L3: QSFDNSLSAYV (SEQ ID NO: 21), and
wherein the anti-PD-1 antibody or antigen-binding fragment thereof comprises a variable light chain region comprising CDRL1 of SEQ ID NO: 95, CDRL2 of SEQ ID NO: 96, and CDRL3 of SEQ ID NO: 97, and a variable heavy chain region comprising CDRH1 of SEQ ID NO: 100, CDRH2 of SEQ ID NO: 101, and CDRH3 of SEQ ID NO: 102.

25. The formulation of claim 1, wherein the anti-ILT4 antibody or antigen binding fragment thereof comprises a heavy chain variable domain comprising: CDR-H1: GYYWS (SEQ ID NO: 16), CDR-H2: EINHSGSTNYNPSLKS (SEQ ID NO:47), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GX₁X₂NRPS, wherein X₁ is N, Q, E or D and X₂ is S or A (SEQ ID NO: 20), and CDR-L3: QSFDNSLSAYV (SEQ ID NO: 21).

26. The formulation of claim 8, wherein the anti-ILT4 antibody or antigen binding fragment thereof comprises a heavy chain variable domain comprising: CDR-H1: GYYWS (SEQ ID NO: 16), CDR-H2: EINHAGSTNYNPSLKS (SEQ ID NO: 48), and CDR-H3: LPTRWVTTRYFDL (SEQ ID NO: 18); and a light chain variable domain comprising: CDR-L1: TGSSSNIGAGYDVH (SEQ ID NO: 19), CDR-L2: GDSNRPS (SEQ ID NO: 52), and CDR-L3: QSFDNSLSAYV (SEQ ID NO: 21).

27. The formulation of claim 1, wherein the anti-ILT4 antibody or antigen binding fragment thereof comprises a heavy chain variable domain comprising the amino acid sequence set forth in SEQ ID NO:57 and a light chain variable domain comprising the amino acid sequence set forth in SEQ ID NO:58.

28. The formulation of claim 8, wherein the anti-ILT4 antibody or antigen binding fragment thereof comprises a heavy chain comprising the amino acid sequence set forth in SEQ ID NO:2 and a light chain comprising the amino acid sequence set forth in SEQ ID NO:7.

29. The formulation of claim 24, wherein the anti-ILT4 antibody or antigen binding fragment thereof comprises a heavy chain comprising the amino acid sequence set forth in SEQ ID NO:80 and a light chain comprising the amino acid sequence set forth in SEQ ID NO:7.

30. The formulation of claim 1, wherein the anti-ILT4 antibody or antigen binding fragment thereof comprises a heavy chain variable domain consisting of the amino acid sequence set forth in SEQ ID NO:57 and a light chain variable domain consisting of the amino acid sequence set forth in SEQ ID NO:58.

31. The formulation of claim 1, wherein the anti-ILT4 antibody or antigen binding fragment thereof comprises a heavy chain consisting of the amino acid sequence set forth in SEQ ID NO:2 and a light chain consisting of the amino acid sequence set forth in SEQ ID NO:7.

32. The formulation of claim 24, wherein the anti-IL T4 antibody or antigen binding fragment thereof comprises a heavy chain consisting of the amino acid sequence set forth in SEQ ID NO:80 and a light chain consisting of the amino acid sequence set forth in SEQ ID NO:7.

33. The formulation of claim 24, wherein the anti-human PD-1 antibody or antigen binding fragment thereof comprises a variable light region which comprises the amino acid sequence set forth in SEQ ID NO: 98, and a variable heavy region which comprises the amino acid sequence set forth in SEQ ID NO: 103.

34. The formulation of claim 1, wherein the anti-human PD-1 antibody or antigen binding fragment thereof comprises a variable light region which comprises the amino acid sequence set forth in SEQ ID NO: 99, and a variable heavy region which comprises the amino acid sequence set forth in SEQ ID NO: 104.

35. The formulation of claim 8, wherein the anti-human PD-1 antibody or antigen binding fragment thereof consists of two light chains and two heavy chains, wherein the two light chains consist of the amino acid sequence set forth in SEQ ID NO:99 and the two heavy chains consist of the amino acid sequence set forth in any one of SEQ ID NOs:104-109.

36. The formulation of claim 24, wherein the anti-human PD-1 antibody is pembrolizumab.

37. The formulation of claim 1, wherein the ratio of anti-ILT4 antibody to the anti-PD-1 antibody is 1:1 or 1:2.

38. The formulation of claim 8, wherein after 9 months at 5° C.:
(i) the % monomer of the anti-ILT4 antibody is ≥95% as determined by size exclusion chromatography;
(ii) the % heavy chain and light chain of the anti-ILT4 antibody is ≥90% as measured by reduced CE-SDS;
(iii) the % heavy chain and light chain of the anti-ILT4 antibody is ≥95% as measured by reduced CE-SDS;
(iv) the % intact IgG of the anti-ILT4 antibody is ≥90% as measured by non-reduced CE-SDS; and/or
(v) the % intact IgG of the anti-ILT4 antibody is ≥95% as measured by non-reduced CE-SDS;

39. (canceled)

40. The formulation of claim 24, wherein after storage at about 3° C. to about 5° C. for up to 9 months:
(i) the % monomer of the anti-ILT4 antibody or antigen-binding fragment thereof is at least about 99% as determined by ultra-performance size exclusion chromatography;
(ii) the turbidity of the formulation is at most about 0.135 as measured by OD₃₅₀₋₅₀₀;
(iii) the % main peak of the anti-ILT4 antibody or antigen-binding fragment thereof is at least about 63%, the % acidic variant of the anti-ILT4 antibody or antigen-binding fragment thereof is at most about 23%, and/or the basic variant of the anti-ILT4 antibody or antigen-binding fragment thereof is at most about 14%, as determined by high performance ion-exchange chromatography;
(iv) the subvisible particle count of particles that are at least 2 μm in size is at most about 3500 as determined by microflow imaging; and/or
(v) the % oxidation of one or more amino acid residues selected from the group consisting of W7, W102, M253, M359, and M429 in the heavy chain of the anti-ILT4 antibody as set forth in SEQ ID NO:2 or 80 is less than about 4%, as determined by reduced peptide mapping.

41. The formulation of claim 1, wherein the anti-PD-1 antibody or antigen-binding fragment thereof is a monoclonal antibody.

42. A method of treating a cancer in a human patient in need thereof, the method comprising administering an effective amount of the formulation of claim 1.

43. The method of claim 42, wherein the cancer is selected from the group consisting of colorectal cancer, esophageal cancer, melanoma, non-small cell lung cancer, ovarian cancer, renal cell cancer, and small cell lung cancer.

44-46. (canceled)


SEQUENCE LISTING (114 sequences)

SEQ ID NO: 1 - protein, 448 aa, synthetic construct
EVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 448 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 217 aa, synthetic construct
ESVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 217 aa, synthetic construct
ESVLTQPPSVSGAPGQRVTISCTGSSSNIGAGYDVHWYQQLPGTAPKLLIYGNANRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 217 aa, synthetic construct
ESVLTQPPSVSGAPGQRVTISCTGSSSNIGAGYDVHWYQQLPGTAPKLLIYGQSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 217 aa, synthetic construct
ESVLTQPPSVSGAPGQRVTISCTGSSSNIGAGYDVHWYQQLPGTAPKLLIYGESNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 217 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 451 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYFDSSGWYKGGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 451 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYFDASGWYKGGAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 451 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYFSSSGWYKGGAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 221 aa, synthetic construct
QSVLTQPSSLSASPGASASLTCTLRSGINVDTYRIHWYQQKPGSPPQYLLRYKSDSDKHQGSGVPSRFSGSKDPSANAGILLISGLQSEDEADYYCAIWYSSTWVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 449 aa, synthetic construct
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVGEWIQLWSPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 214 aa, synthetic construct
DIQMTQSPSSVSASVGDRVTITCRASQGISSWLAWYQQKPGKAPKFLIYAASSLQSGVPSKFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 456 aa, synthetic construct
EVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCARAGPLYTIFGVVIIPDNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 219 aa, synthetic construct
ESVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSGVVFGGGTQLIILGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 5 aa, synthetic construct
GYYWS

SEQ ID NO: 17 - protein, 16 aa, synthetic construct
  VARIANT 5: note = Serine or Alanine
EINHXGSTNYNPSLKS

SEQ ID NO: 18 - protein, 13 aa, synthetic construct
LPTRWVTTRYFDL

SEQ ID NO: 19 - protein, 14 aa, synthetic construct
TGSSSNIGAGYDVH

SEQ ID NO: 20 - protein, 7 aa, synthetic construct
  VARIANT 2: note = Asparagine, Glutamine, Glutamic acid or Aspartic acid
  VARIANT 3: note = Serine or Alanine
GXXNRPS

SEQ ID NO: 21 - protein, 11 aa, synthetic construct
QSFDNSLSAYV

SEQ ID NO: 22 - protein, 5 aa, synthetic construct
SYAIS

SEQ ID NO: 23 - protein, 17 aa, synthetic construct
GIIPIFGTANYAQKFQG

SEQ ID NO: 24 - protein, 15 aa, synthetic construct
  VARIANT 3: note = Aspartic acid or Serine
  VARIANT 4: note = Serine or Alanine
YFXXSGWYKGGAFDI

SEQ ID NO: 25 - protein, 14 aa, synthetic construct
TLRSGINVDTYRIH

SEQ ID NO: 26 - protein, 11 aa, synthetic construct
YKSDSDKHQGS

SEQ ID NO: 27 - protein, 9 aa, synthetic construct
AIWYSSTWV

SEQ ID NO: 28 - protein, 5 aa, synthetic construct
SYAMH

SEQ ID NO: 29 - protein, 17 aa, synthetic construct
VISYDGSNKYYADSVKG

SEQ ID NO: 30 - protein, 13 aa, synthetic construct
VGEWIQLWSPFDY

SEQ ID NO: 31 - protein, 11 aa, synthetic construct
RASQGISSWLA

SEQ ID NO: 32 - protein, 7 aa, synthetic construct
AASSLQS

SEQ ID NO: 33 - protein, 9 aa, synthetic construct
QQYNSYPPT

SEQ ID NO: 34 - protein, 5 aa, synthetic construct
ELSMH

SEQ ID NO: 35 - protein, 17 aa, synthetic construct
GFDPEDGETIYAQKFQG

SEQ ID NO: 36 - protein, 20 aa, synthetic construct
AGPLYTIFGVVIIPDNWFDP

SEQ ID NO: 37 - protein, 14 aa, synthetic construct
TGSSSNIGAGYDVH

SEQ ID NO: 38 - protein, 7 aa, synthetic construct
GNSNRPS

SEQ ID NO: 39 - protein, 13 aa, synthetic construct
QSYDSSLSGSGVV

SEQ ID NO: 40 - skipped

SEQ ID NO: 41 - protein, 19 aa, unidentified
MEWSWVFLFFLSVTTGVHS

SEQ ID NO: 42 - protein, 20 aa, unidentified
MSVPTQVLGLLLLWLTDARC

SEQ ID NO: 43 - skipped

SEQ ID NO: 44 - protein, 451 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 217 aa, synthetic construct
QSVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - skipped

SEQ ID NO: 47 - protein, 16 aa, synthetic construct
EINHSGSTNYNPSLKS

SEQ ID NO: 48 - protein, 16 aa, synthetic construct
EINHAGSTNYNPSLKS

SEQ ID NO: 49 - protein, 7 aa, synthetic construct
GNSNRPS

SEQ ID NO: 50 - protein, 7 aa, synthetic construct
GQSNRPS

SEQ ID NO: 51 - protein, 7 aa, synthetic construct
GESNRPS

SEQ ID NO: 52 - protein, 7 aa, synthetic construct
GDSNRPS

SEQ ID NO: 53 - protein, 7 aa, synthetic construct
GNANRPS

SEQ ID NO: 54 - protein, 7 aa, synthetic construct
GQANRPS

SEQ ID NO: 55 - protein, 7 aa, synthetic construct
GEANRPS

SEQ ID NO: 56 - protein, 7 aa, synthetic construct
GDANRPS

SEQ ID NO: 57 - protein, 121 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSS

SEQ ID NO: 58 - protein, 111 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVL

SEQ ID NO: 59 - protein, 10 aa, unidentified
LYREKKSASW

SEQ ID NO: 60 - protein, 7 aa, unidentified
TRIRPEL

SEQ ID NO: 61 - protein, 4 aa, unidentified
NGQF

SEQ ID NO: 62 - protein, 8 aa, unidentified
HTGRYGCQ

SEQ ID NO: 63 - protein, 121 aa, synthetic construct
EVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSS

SEQ ID NO: 64 - protein, 124 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYFDSSGWYKGGAFDIWGQGTMVTVSS

SEQ ID NO: 65 - protein, 124 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYFDASGWYKGGAFDIWGQGTLVTVSS

SEQ ID NO: 66 - protein, 124 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYFSSSGWYKGGAFDIWGQGTLVTVSS

SEQ ID NO: 67 - protein, 122 aa, synthetic construct
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVGEWIQLWSPFDYWGQGTLVTVSS

SEQ ID NO: 68 - protein, 129 aa, synthetic construct
EVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCARAGPLYTIFGVVIIPDNWFDPWGQGTLVTVSS

SEQ ID NO: 69 - protein, 121 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSS

SEQ ID NO: 70 - protein, 111 aa, synthetic construct
ESVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVL

SEQ ID NO: 71 - protein, 111 aa, synthetic construct
ESVLTQPPSVSGAPGQRVTISCTGSSSNIGAGYDVHWYQQLPGTAPKLLIYGNANRPSGVPDRFSVSKSGASASLAITGLQAEDEADYYCQSFDNSLSAYVFGGGTQLTVL

SEQ ID NO: 72 - protein, 111 aa, synthetic construct
ESVLTQPPSVSGAPGQRVTISCTGSSSNIGAGYDVHWYQQLPGTAPKLLIYGQSNRPSGVPDRFSVSKSGASASLAITGLQAEDEADYYCQSFDNSLSAYVFGGGTQLTVL

SEQ ID NO: 73 - protein, 111 aa, synthetic construct
ESVLTQPPSVSGAPGQRVTISCTGSSSNIGAGYDVHWYQQLPGTAPKLLIYGESNRPSGVPDRFSVSKSGASASLAITGLQAEDEADYYCQSFDNSLSAYVFGGGTQLTVL

SEQ ID NO: 74 - protein, 115 aa, synthetic construct
QSVLTQPSSLSASPGASASLTCTLRSGINVDTYRIHWYQQKPGSPPQYLLRYKSDSDKHQGSGVPSRFSGSKDPSANAGILLISGLQSEDEADYYCAIWYSSTWVFGGGTQLTVL

SEQ ID NO: 75 - protein, 107 aa, synthetic construct
DIQMTQSPSSVSASVGDRVTITCRASQGISSWLAWYQQKPGKAPKFLIYAASSLQSGVPSKFSGSGSGTDFTLTISSLQPEDFATYYCQQYNSYPPTFGGGTKVEIK

SEQ ID NO: 76 - protein, 113 aa, synthetic construct
ESVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSGVVFGGGTQLIIL

SEQ ID NO: 77 - protein, 111 aa, synthetic construct
QSVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVL

SEQ ID NO: 78-90 - skipped (13 entries)

SEQ ID NO: 91 - protein, 15 aa, synthetic construct
YFDSSGWYKGGAFDI

SEQ ID NO: 92 - protein, 15 aa, synthetic construct
YFSSSGWYKGGAFDI

SEQ ID NO: 93 - protein, 15 aa, synthetic construct
YFDASGWYKGGAFDI

SEQ ID NO: 94 - protein, 15 aa, synthetic construct
YFSASGWYKGGAFDI

SEQ ID NO: 95 - protein, 15 aa, synthetic construct
RASKGVSTSGYSYLH

SEQ ID NO: 96 - protein, 7 aa, synthetic construct
LASYLES

SEQ ID NO: 97 - protein, 9 aa, synthetic construct
QHSRDLPLT

SEQ ID NO: 98 - protein, 111 aa, synthetic construct [sequence omitted]

SEQ ID NO: 99 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 5 aa, synthetic construct
NYYMY

SEQ ID NO: 101 - protein, 17 aa, synthetic construct
GINPSNGGTNFNEKFKN

SEQ ID NO: 102 - protein, 11 aa, synthetic construct
RDYRFDMGFDY

SEQ ID NO: 103 - protein, 120 aa, synthetic construct [sequence omitted]

SEQ ID NO: 104 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 11 aa, synthetic construct
RASQSVSSYLA

SEQ ID NO: 106 - protein, 7 aa, synthetic construct
DASNRAT

SEQ ID NO: 107 - protein, 9 aa, synthetic construct
QQSSNWPRT

SEQ ID NO: 108 - protein, 107 aa, synthetic construct [sequence omitted]

SEQ ID NO: 109 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 5 aa, synthetic construct
NSGMH

SEQ ID NO: 111 - protein, 17 aa, synthetic construct
VIWYDGSKRYYADSVKG

SEQ ID NO: 112 - protein, 4 aa, synthetic construct
NDDY

SEQ ID NO: 113 - protein, 113 aa, synthetic construct [sequence omitted]

SEQ ID NO: 114 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK